(19) United States
(12) Patent Application Publication
(10) Pub. No.: US 2025/0049911 A1
(43) Pub. Date: Feb. 13, 2025

(54) IMPLANTABLE SCAFFOLDS FOR IMMUNOTHERAPEUTIC AND OTHER USES

(71) Applicant: The Regents of the University of California, Oakland, CA (US)

(72) Inventors: Mohammad Mahdi HASANI-SADRABADI, Los Angeles, CA (US); Song LI, Beverly Hills, CA (US); Manish J. BUTTE, Santa Monica, CA (US)

(73) Assignee: The Regents of the University of California, Oakland, CA (US)

(21) Appl. No.: 18/719,643

(22) PCT Filed: Dec. 15, 2022
(86) PCT No.: PCT/US22/53040

§ 371 Date: Jun. 13, 2024

Related U.S. Application Data
(60) Provisional application No. 63/290,594, filed on Dec. 16, 2021.

(51) Int. Cl. A61K 39/215; A61K 38/19; A61K 38/20; A61K 39/00; A61K 47/61; A61K 47/62; A61K 47/69; A61M 5/178

(52) U.S. Cl. CPC A61K 39/215; A61K 38/195; A61K 38/2013; A61K 38/2046; A61K 38/2086; A61K 39/0011; A61K 47/61; A61K 47/62; A61K 47/6927; A61M 5/178; A61K 2039/54; A61K 2039/55555

(57) ABSTRACT

An implantable or injectable scaffold comprising an antigen, a T memory cell inducer, and other optional components is provided for use in enhancing the immune response to the antigen, in particular for subjects who are elderly, immunosenescent and/or immunocompromised, or undergoing cancer therapy. Coronavirus SARS-CoV-2 has caused millions of confirmed cases and hundreds of thousands of deaths. However, there is no effective drug treatment, and vaccine is in great need to control the spread of such highly infectious virus.

### CROSS-REFERENCE TO RELATED APPLICATIONS

This application claims priority to U.S. Provisional Patent Application Ser. No. 63/290,594, filed Dec. 16, 2021, which is incorporated herein by reference in its entirety.

### FIELD OF INTEREST

The present disclosure is directed to compositions for eliciting or enhancing the immune response for the purpose of vaccination for therapeutic or preventative purposes.

### BACKGROUND

Coronavirus SARS-CoV-2 has caused millions of confirmed cases and hundreds of thousands of deaths. However, there is no effective drug treatment, and vaccine is in great need to control the spread of such highly infectious virus. A number of public and private initiatives are developing a variety of potential vaccines, including inactivated viruses, viral vector-based vaccine, cellular vaccines, RNA vaccines and protein-antigen based vaccines. Recent studies suggest that the Spike protein of SARS-CoV-2, which mediates the endocytosis of the virus by the infected cells, can be a vaccine candidate to induce T cell responses and antibody production.

Most elderly people cannot generate effective immune responses to gain long-term immunity due to immunosenescence. The high susceptibility and death rate of the elderly due to SARS-CoV-2 infection presents a major challenge to our healthcare system. An aging immune system is well documented as the cause of increased infection rates in elderly people, which also renders vaccination less effective in the elderly due to the decline of innate and adaptive immune system. Aging immune systems usually fails to induce long-lived memory T cells; thus, in order to protect the elderly population, it is critical to boost the generation of T memory stem cells (TMSCs) that can provide long-term immunity. TMSCs are unique subsets of memory lymphocytes endowed with the stem cell-like ability to self-renew and the multipotent capacity to reconstitute the entire spectrum of memory and effector T cells. Maintenance of long-lived T cell is crucial because increased T cell loss after the peak period is a main reason of diminishing memory in the elderly. Cumulative evidence in mice, nonhuman primates as well as humans indicate that TMSCs could be leveraged therapeutically to enhance the efficacy of vaccines for infectious diseases and cancers. Compared with other memory T cell subsets, TMSCs demonstrates a faster response to antigen stimulation which will make the immunization more effective. In addition, TMSC preferentially survive after the elimination of antigens, stably persist for a long period of time, and can reconstitute the entire peripheral T cell population with a small number of cells. For instance, it has been reported that a yellow fever virus-specific TMSC population could stably maintain for more than 25 years, while other groups also identified TMSC in the circulation possessing self-renewal capacity and clonal longevity, which are necessary for sustaining long-term immunological memory. These studies supported that TMSC should play an important role in sustaining peripheral T cell homeostasis; moreover, recent studies highlight the importance of this cell population in elderly population. Although there is no targeted approach to boost up formation of TMSCs during the regular vaccination, evidence suggests that, in the elderly, successful generation of TMSC during the vaccination can significantly improve the cellular immunity in the elderly.

It is towards the development of a system that can engage with tissue resident immune cells and enhance long-term immunity that the disclosure is directed.

### SUMMARY

In one aspect, a microparticle is provided comprising: at least one immunogen; and at least one T memory stem cell (TMSC) inducer.

In some embodiments, the immunogen is a viral, bacterial, protozoan, helminthic, or cancer antigen. In some embodiments, the cancer antigen is from a solid tumor.

In some embodiments, the at least one T memory stem cell (TMSC) inducer is TWS119.

In some embodiments, the microparticle further comprises at least one immunostimulatory cytokine. In some embodiments, the least one immunostimulatory cytokine is IL-2, IL-7 or IL-15, or any combination thereof.

In some embodiments, the microparticle further comprises a T cell activator. In some embodiments, the T cell activator is anti-CD3 antibodies and/or anti-CD28 antibodies, or the combination thereof.

In any of the foregoing embodiments, the microparticle further comprise an adjuvant such as poly (I:C), CpG, alum, or any combination thereof.

In any of the foregoing embodiments, the microparticle further comprise an RGD peptide.

In any of the foregoing embodiments, the microparticle further comprise a chemokine.

In certain embodiments, the chemokine is CCL21, CCL19, CCL17, CCL22 or any combination thereof.

In any of the foregoing embodiments, the microparticle further comprise heparin.

In any of the foregoing embodiments, the microparticle comprise a polymer scaffold such as alginate, hyaluronic acid, polycaprolactone, poly(lactic acid), or PLGA. In certain embodiments, the alginate is cross-linked with calcium.

In some embodiments, the TMSC inducer is provided in a nanoparticle within the microparticle. In some embodiments, the nanoparticle comprises PLGA.

In any of the foregoing embodiments, the microparticles comprise a porous scaffold.

In some embodiments, the porous scaffold comprises anti-CD3 or anti-CD28 antibodies bound to or cross-linked to heparin. In some embodiments, the cross-link is by NHS/EDC.

In any of the foregoing embodiments, the microparticle comprise RGD peptide conjugated to alginate via NHS/EDC.

In any of the foregoing embodiments, the microparticles have an average size of about 0.4 μm to about 50 μm.

In any of the foregoing embodiments, the microparticle have an average size of about 200 μm to about 800 μm. In some embodiments, the microparticles have an average pore size of about 20 μm to about 100 μm.

In any of the foregoing embodiments, the microparticles comprise a stiffness of from about 0.5-250 kPa.

In an aspect, a pharmaceutical composition comprising the microparticles of any one of the foregoing embodiments. In some embodiments, the pharmaceutical composition comprises a vehicle, excipient, vehicle, adjuvant, immune cells, or any combination thereof.

In some embodiments, the adjuvant is alum, CpG, poly (I:C), or any combination thereof.

In some embodiments, the pharmaceutical composition is formulated for subcutaneous injection.

In an aspect, a method is provided for immunizing a subject to an antigen comprising administering to the subject a pharmaceutical composition of any one of the foregoing embodiments or a microparticle of any one of the foregoing embodiments. In some embodiments, the subject is elderly, has immunosenescence, is immunocompromised, or is undergoing cancer therapy. In some embodiments, the immunizing is for therapeutic use. In some embodiments, the immunizing is for preventative use.

### BRIEF DESCRIPTION OF THE FIGURES

The subject matter disclosed herein is particularly pointed out and distinctly claimed in the concluding portion of the specification. The disclosure, however, both as to organization and method of operation, together with objects, features, and advantages thereof, may best be understood by reference to the following detailed description when read with the accompanying drawings in which:

FIG. 1 depicts the mechanism of action of artificial antigen presenting cells (aAPC) disclosed herein. (A) After subcutaneous injection of artificial antigen presenting cells (aAPCs) (engineered biomaterial vaccine), (B) viral-related components (Spike peptides) will be released from and uptake by tissue-resident dendric cells and macrophages. Presence of these antigens in addition to sustained release of cytokines and inhibitor as well as conjugated (anti-CD3/28) cues via aAPCs will drive tissue resident naïve T cells toward formation of antigen specific T memory stem cells (TMSCs). (C) Matured immune cells will then migrated toward the draining lymph nodes (dLNs). In dLNs, adaptive immunity will form via antigen specific expansion of T and B cells. This will cause induction in humoral as well as cellular immunity.

FIG. 2 is a schematic representation of artificial antigen presenting cells (aAPCs). These monodisperse particles were made using microfluidic platforms or membrane emulsification technique in size range of 0.4-50 um. Wide range of natural and synthetic polymers can be used as core materials

FIG. 3 is an example of aAPCs formulation. Alginate conjugated with heparin will be used as core materials. Alginate is a biocompatible polysaccharide polymer which can be crosslinked via use of divalent cations like Calcium and Barium or via chemical crosslinking. Presence of heparin will increase the affinity of cytokines to the particles in natural way as it represents glycosaminoglycans (GAG). This Alginate-Heparin conjugate was tested toward encapsulation and sustained release of target cytokines (IL-2, IL-7, and IL-15). Due to hydrophobic nature of TWS119, we utilized PLGA (50:50 DL-lactide/Glycolide, PURASORB PDLG 5004A) to encapsulate this small molecule inhibitor. PLGA nanoparticles (NPs) in size range of 100-300 nm were formed and the mixed with Alginate-Heparin prior to forming microparticle hydrogels. The microparticles will be surface functionalized with anti-CD3 and anti-CD28 to provide active engagement with T cells. Adjuvants (like CpG, Poly(I:C)) will be added to the formulation (encapsulated in microparticles) for in vivo experiments. Addition of adjuvant is necessary to increase immunogenicity at the injection site.

FIG. 4 depicts confocal micrograph of microparticles coated with fluorescent anti-CD3 antibody showing even distribution of antibodies on the microparticles' surface.

FIG. 5 depicts a scanning electron microscopy (SEM) picture demonstrating the interactions between primary T cells and aAPCs.

FIG. 6 shows (A) cumulative release of small molecule inhibitor (TWS119) from PLGA loaded encapsulated in aAPCs. (B) Cumulative release of interleukin cytokine (here IL-2) from Alginate and Alginate-Heparin based aAPCs as assessed by ELISA kit.

FIG. 7 shows (Top) Representative brightfield microscopy images of primary mouse T cells cultured with aAPCs modified with various density of anti-CD3 and anti-CD28. Here 5 um aAPCs were used which are based on Alginate-Heparin and loaded with IL-2. No TWS119 is presented here.

FIG. 8 shows that T cell factor (TCF) 1 and lymphoid enhancer-binding factor (LEF) 1 are downstream transcription factors of the Wnt/β-catenin signaling pathway. Based on the literatures indicate that Wnt signaling might regulate the maturation of T cells. Expression of LEF1 and TCF7, decreases with progressive differentiation of CD8+ T cells from naïve (N)→central memory (CM)→effector memory (EM) in humans and mice. TWS119 can activate Wnt signaling in primary CD8+ T cells during the polyclonal activation and induce formation of T memory stem cells (TSCMs). Co-presentation of IL-7 and IL-15 can help further expansion of TSCMs in culture.

FIG. 9 shows that aAPCs delivering TWS119 can activate Wnt signaling in primary CD8+ T cells. Naive CD8+ T cells were isolated from wild-type B6 mice and then incubated with indicated formulations. Quantitative RT-PCR analysis of the expression of TCF7 and LEF1 in CD8+ T cells treated with various formulations. Data are represented as mean±SD. All data are representative of at 3-5 independently performed experiments.

FIG. 10 depicts gating strategy showing population of TSCMs in naïve CD4+(Top panels) and CD8+(Lower panels) T cells. TSCMs are identified as CD62L+CD44−Sca-1+CD4/CD8 T cells. Less than 1% of naïve T cells are TSCMs.

FIG. 11 shows treatment of naïve CD8+ T cells with TWS119 will limit the proliferation as studied by CFSE proliferation staining.

FIG. 12 depicts gating strategy showing population of TSCMs in naïve T cells treated with TWS119 containing aAPCs. Naïve (TN), central memory (CM), and effector memory (EM) T cell population were shown. CD4+ (Top panels) and CD8+(Lower panels) T cells. TSCMs are identified as CD62L+CD44−Sca-1+CD4/CD8 T cells. More than 85% of naïve T cells can be differentiated into TSCMs.

FIG. 13 shows Quantification of TMSCs in CD8+ T cells based on surface markers after four days of co-culturing with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of anti-CD3/anti-CD28 antibodies conjugated on surface of microparticles.

FIG. 14 shows Intracellular expression of IL-2 and IFN-g can be used as cellular markers to identify induced TSCMs.

FIG. 15 shows Intracellular expression of IFN-g was used as cellular markers to quantify induced TSCMs. Naïve T cells were co-cultured with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of anti-CD3/anti-CD28 antibodies conjugated on surface of microparticles.

FIG. 16 shows Intracellular expression of IL-2 was used as cellular markers to quantify induced TSCMs. Naïve T cells were co-cultured with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of anti-CD3/anti-CD28 antibodies conjugated on surface of microparticles.

FIG. 17 depicts flow cytometry showing the difference between population of SCA-1+(CD62L+CD44−CD8+) T cells in naïve cells vs. induced TSCMs.

FIG. 18 depicts Flow cytometry showing the difference between population of SCA-1+CD122+(CD62L+CD44−CD8+) T cells after being treated with aAPCs contained either TWS118 (aAPC-T) or TWS118+IL-7/IL-15 (aAPC-TC) for four days. In order to induce specificity against SARS-CoV-2 (Severe Acute Respiratory Syndrome-related coronavirus 2), the aAPCs were added to solenocyte while loaded with SARS-CoV-2 Spike Glycoprotein peptide pool. This pool includes 316 peptides (Genscript) derived from a peptide scan (15mers with 11 aa overlap) through the entire Spike glycoprotein (Protein ID: P0DTC2) of SARS-CoV-2. Antigen-specific TSCMs can be identified in SCA-1+CD122+ quadrant.

FIG. 19 shows the quantification of flow cytometry results showing the formation of polyclonal, SCA-1+, (Top panels) and SARS-CoV-2 specific, SCA-1+CD122+, (Lower panels) TMSCs. Naïve T cells being treated with aAPCs contained either TWS118 (aAPC-T) or TWS118+IL-7/IL-15 (aAPC-TC) for four days. In order to induce specificity against SARS-CoV-2, the aAPCs were added to solenocyte while loaded with SARS-CoV-2 Spike Glycoprotein peptide pool. This pool includes 316 peptides (Genscript) derived from a peptide scan (15mers with 11 aa overlap) through the entire Spike glycoprotein (Protein ID: P0DTC2) of SARS-CoV-2.

FIG. 20 depicts identification of TSCM cells in human blood. Plots show the gating strategy to identify CD95+CXCR3+TSCM cells in CD4+ and CD8+ T cells. Flow cytometry analysis of human PBMCs without any treatment or after being treated with aAPC-TC. Numbers indicate the percentage of cells in the indicated gates. T cell subsets were defined as follows: TN cells: CD3+CD8+(or CD4+) CD45RO−CCR7+CXCR3−CD95− and TSCM cells: CD3+CD8+(or CD4+)CD45RO−CCR7+CXCR3+CD95+.

FIG. 21 shows quantification of TMSCs in human CD8+ T cells based on surface markers after four days of co-culturing with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of human anti-CD3/anti-CD28 antibodies conjugated on surface of microparticles.

FIG. 22 depicts quantification of TMSCs in human CD4+ T cells based on surface markers after four days of co-culturing with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of human anti-CD3/anti-CD28 antibodies conjugated on surface of microparticles.

FIG. 23 is a plot showing how change in signal intensity delivered by aAPCs can tune the formation of TMSCs in human CD8+ T cells. Presence of TWS119 is essential for such an induction while co-delivery of IL-7/IL-15 can improve the efficiency and sustain the effect.

FIG. 24 is a schematic drawing showing how change in signal intensity delivered by aAPCs can tune the activation of human T cells (plots in red) or modulate formation of TSCMs. Presence of TWS119 is essential for differentiation of naïve T cells toward TSCMs while co-delivery of IL-7/IL-15 can improve the efficiency and sustain the effects.

FIG. 25 shows vaccination timeline in experimental animal. Here wild-type (C57BL/6) mice were used for single dose subdermal vaccination. Blood collected from vaccinated animal at 7-day timepoint used for SARS-CoV-2 IgM detection. Blood collected at week 4 and week 8, were used to measure SARS-CoV-2 IgG, IgG1, and IgG2b titers. Lung and spleen tissues were extracted and digested into single cell to analyze cellular response after re-stimulation with r SARS-CoV-2 Spike peptides.

FIG. 26 shows the ELISA method was used to detect IgM, IgG and IgG subunits (IgG1 and IgG2b).

FIG. 27 depicts vaccination with aAPCs elicits potent SARS-CoV-2 Spike humoral response in C57BL/6J. Mice were immunized on days 0 with indicated formulations and humoral responses analyzed on day 7 by ELISA for IgM.

FIG. 28 depicts vaccination with aAPCs elicits SARS-CoV-2 Spike humoral response in C57BL/6J. Mice were immunized on days 0 with indicated formulations and humoral responses analyzed on day 28 by ELISA for IgG.

FIG. 29 depicts vaccination with aAPCs elicits SARS-CoV-2 Spike humoral response in C57BL/6J. Mice were immunized on days 0 with indicated formulations and humoral responses analyzed on day 28 by ELISA for IgG1 (Th2) and IgG2b (Th1).

FIG. 30 depicts vaccination with aAPCs elicits potent Th1 response against SARS-CoV-2 Spike in C57BL/6J. Mice were immunized on days 0 with indicated formulations and humoral responses analyzed on day 28 by ELISA for IgG1 (Th2) and IgG2b (Th1). The ratio of IgG2b to IgG1 titer indicated a strong bias towards Th1 for aAPCp immunized animals specially when TWS119 inhibitor was presented (aAPCp-i). Soluble adjuvants Alum, poly(I:C), and CpG produced a balanced Th1/Th2 profile or Th2-dominant response while aAPCs formulations due to providing sustained release of their cargos as well as providing active engagement with local immune cells can switch the response toward Th1.

FIG. 31 depicts vaccination with aAPCs elicits potent Th1 response against SARS-CoV-2 Spike in young (3-month-old) vs. aged (13-month-old) C57BL/6J mice. Mice were immunized on days 0 with indicated formulations and humoral responses analyzed on day 28 by ELISA for IgG1 (Th2) and IgG2b (Th1). The ratio of IgG2b to IgG1 titer indicated a strong bias towards Th1 for aAPCp immunized animals specially when TWS119 inhibitor was presented (aAPCp-i). Soluble adjuvants Alum, poly(I:C), and CpG produced a balanced Th1/Th2 profile or Th2-dominant response while aAPCs formulations due to providing sustained release of their cargos as well as providing active engagement with local immune cells can switch the response toward Th1. Similar trend can be seen in aged animals.

FIG. 32 shows the change in mass of vaccine draining lymph node is evaluated. Successful vaccination can result in accumulation and expansion of antigen-specific immune cells in draining lymph node close to vaccination site. Mean±SD (n=3).

FIG. 33 shows the change in mechanical stiffness of vaccine draining lymph node is evaluated. Successful vaccination can result in accumulation and expansion of antigen-specific immune cells in draining lymph node close to vaccination site. Mean±SD (n=3).

FIG. 34 depicts vaccination with aAPCs elicits enhanced SARS-CoV-2 Spike-specific T cell responses in C57BL/6J. Mice were immunized on day 0 and T cell responses analyzed on day 28. peripheral blood cells collected at week 4 and were analyzed by flow cytometry after being stimulated with SARS-CoV-2 Spike peptide pool. This pool includes 316 peptides (Genscript) derived from a peptide scan (15mers with 11 aa overlap) through the entire Spike glycoprotein (Protein ID: P0DTC2) of SARS-CoV-2. This stimulation can trigger intracellular cytokine production to detect antigen-specific T cell responses. Shown are frequencies of IFNγ, T cells among peripheral blood CD8+ T cells.

FIG. 35 shows the mechanism of action of the 3D scaffolds in training tissue resident immune cells to induce robust and prolonged immunity against SARS-CoV-2 infection.

FIGS. 36A-36H show the biological, biochemical and biophysical properties of aAPCs that induce the formation of human TMSCs in vitro. FIG. 36A: Schematic summary of the designed aAPCs. Alginate-heparin conjugated was used as the matrix to form aAPCs. TWS119 was encapsulated in PLGA nanoparticles and then mixed with Spike peptides, cytokines, and alginate-heparin to form aAPCs. Following the crosslinking, surface of microparticles was functionalized with anti-CD3 and anti-CD28 antibodies. FIG. 36B: Confocal fluorescent microscopy was used to reconstruct 3D structure of alginate-based microparticles. Anti-CD3 antibodies on the surface of microparticles were stained with FITC-CD3 (left panel). Interaction of primary naïve human CD8+ T cells (in green) with aAPCs (in red) was demonstrated 2 hours after culture (middle panel). T cells strongly engaged with the aAPCs (stained for CD3, in green) and caused clustering of the lymphocyte function-associated antigen-1 (LFA-1) (stained in cyan) on the surface of T cells to form immune synapsis (right panel). FIG. 36C: Cumulative in vitro release of Spike peptides, TWS119, IL-2, IL-7, and IL-15 over time at 37° C. under gentle shaking (n=4). FIG. 36D: TMSC induction in vitro was modulated by the biochemical and biological signals of aAPCs (5 μm, 20 kPa). Flow cytometry analysis of primary human naïve CD8⁺ T cells before and after 4 days of stimulation by aAPCs with various formulations in the presence or absence of TWS119. For these experiments, TWS119 was encapsulated in PLGA nanoparticles and then being loaded in aAPCs formulations. Signal intensities of antibodies were estimated by measuring approximate density of anti-CD3/anti-CD28 on the surface of aAPCs and categorized as low, med(ium) and Hi(gh) (n=5). FIG. 36E: Presence of TWS119/IL-7/IL-15 in formulation (aAPCT+) can significantly improve TMSCs expansion in vitro (n=5). FIG. 36F: aAPCs with diameters ranging from 400 nm to 250 μm were used to treat T cells for 4 days to maximize the TMSC formation in vitro (n=5). FIG. 36G: TMSC induction was modulated by the mechanical properties of aAPCs. The aAPCs (5 m in diameter) with various stiffness ranging from 10-200 kPa was tested (n=5). FIG. 36H: Quantitative RT-PCR analysis of LEF1 and TCF7 expressions in human CD8⁺ T cells treated with various formulations for 48 hours. Each column indicates the results of an independent experiment (n=4). Ctrl: IL-2 only; aAPC: alginate-heparin/IL-2 microparticles (5 m, 20 kPa) with medium level of anti-CD3/anti-CD28 surface antibodies; aAPCT: aAPC with TWS119 PLGA nanoparticles; aAPCT+: aAPCT+IL7/IL-15; Dyn: dynabead with soluble IL-2; Dyn/T: Dyn with soluble IL-2/TWS119. The results were analyzed by using one-way ANOVA with post-hoc analysis. Statistical significance is indicated by the calculated p-values.

FIGS. 37A-37E show biomaterial-based aAPCs can induce the formation of murine TMSCs in vitro. FIG. 37A: Quantitative RT-PCR analysis of LEF1 and TCF7 expression in murine CD8⁺ T cells treated with various stimuli after 48 hours. Ctrl: IL-2 only; aAPC: alginate-heparin/IL-2 microparticles (5 μm, 20 kPa) with medium level of anti-CD3/anti-CD28 surface antibodies; aAPCT: aAPC with TWS119 PLGA nanoparticles; aAPCT+: aAPCT+IL-7/IL-15; Dyn: dynabead with soluble IL-2; Dyn/T: Dyn with soluble IL-2/TWS119. FIG. 37B: Quantitative RT-PCR analysis of LEF1 and TCF7 expression in murine CD8⁺ T cells over time. FIG. 37C: TMSC induction in vitro is modulated by the biological and biochemical properties of aAPCs. Flow cytometry analysis of primary murine naïve CD8⁺ T cells before and 4 days after stimulation with aAPCs with various formulations in the presence or absence of 5 μM TWS119. Signal intensities of antibodies were categorized as low, med(ium) and Hi(gh) by measuring densities of anti-CD3/anti-CD28 on the surface of aAPCs. Secretion of IFN-γ (FIG. 37D) and IL-2 (FIG. 37E) from T cells was measured by ELISA 4 days post treatment. The presented data are expressed as mean±SD (n=5). The results were analyzed by using one-way ANOVA with post-hoc analysis. Statistical significance is indicated by the calculated p-values. significant: p<0.05, very significant: p<0.01 and extremely significant: p<0.001.

FIGS. 38A-38G show biomaterial-based vaccines can induce TMSC formation in vivo. FIG. 38A: C57BL/6 mice were immunized on days 0 with indicated formulations. Stiffening of vaccine-dLNs was monitored over time as a macroscopic evidence of vaccination efficacy. Area under the curve (AUC) was calculated for animal treated with various formulations. (n=3) FIG. 38B-38D: To study TMSC formation in vivo, C57BL/6 mice were immunized on days 0 with aAPC, aAPCT or Vax-T, or immunized on day 0 and 14 with control vaccine formulations without (Soluble) or with (Soluble-T) TWS119. FIG. 38B: On day 21, the draining lymph nodes were extracted and population of TMSCs (CD8⁺CD62L⁺CD44⁻Sca-1⁺ T cells) were identified using flow cytometry. Isolated lymph node cells were sorted for naïve-like CD8+ T cell population (CD62L⁺CD44⁻) and restimulated with overlapping peptide pool for 3 days and secretion of IFN-γ (FIG. 38C) and IL-2 (FIG. 38D) were quantified. (n=5). Population of leukocytes (CD45⁺), dendritic cells (CD11c⁺MHCII⁺), macrophages (F4/80⁺), T cells (CD3⁺) as well as TMSCs (CD8⁺CD62L⁺CD44⁺Sca-1⁺) were assessed in vaccine injection sites (FIG. 38E) and draining lymph nodes (FIG. 38F) at three different timepoints. FIG. 38G: Total number of cells that ingested TWS119-loaded Rhodamine-labeled PLGA nanoparticles in Vax-T formulation were also traced at the injection site and dLNs. The presented data are expressed as mean±SD (n=5). Sham: Saline injection; Soluble: SARS-CoV-2 peptides/IL-2/CpG; Soluble-T: Soluble with TWS119. aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 PLGA nanoparticles; Vax-T: aAPCT without anti-CD3/anti-CD28 surface antibodies. IL-2: 100 U/injection; CpG 50 μg/injection; SARS-CoV-2 peptides 10 μg/injection. The results were analyzed by using one-way ANOVA with post-hoc analysis. Statistical significance is indicated by the calculated p-values. Significant: p<0.05, very significant: p<0.01 and extremely significant: p<0.001.

FIGS. 39A-39F show vaccination with aAPCs/Vax-T promoted potent T cell specific responses in young and aged mice against SARS-CoV-2. To study formation and function of SARS-CoV-2 specific T cell response in vivo, young and aged C57BL/6 mice were immunized on days 0 with aAPCT and on day 0 and 14 with control vaccine formulations without (Soluble) or with (Soluble-T) TWS119. Eight weeks post vaccination, spleen cells were stimulated with overlapping Spike peptides and assayed by flow cytometry for intracellular cytokines production to detect antigen-specific T cell responses. Shown are frequencies of IFN-γ, TNF-α, IL-2 and triple-positive T cells among CD8⁺ T cells in young (FIG. 39A) and aged mice (FIG. 39B). Secretion of pro-inflammation (IFN-γ, TNF-α, and IL-2) and anti-inflammation (IL-4) cytokines from splenocyte of vaccinated mice after being restimulated with overlapping Spike peptides were measured in young (FIGS. 39C-39D) and aged mice (FIGS. 39E-39F). Sham: Saline injection; Soluble: SARS-CoV-2 peptides/IL-2/CpG; Soluble-T: Soluble with TWS119. aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 nanoparticles; Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=5). The results were analyzed by using one-way ANOVA with post-hoc analysis. Statistical significance is indicated by the calculated p-values. Significant: p<0.05, very significant: p<0.01 and extremely significant: p<0.001.

FIGS. 40A-40J show vaccination with aAPCs/Vax-T promoted potent humoral responses against SARS-CoV-2 in young and aged mice. FIG. 40A: Timeline of experimental procedure. To study humoral responses against SARS-CoV-2 antigens, C57BL/6 mice were immunized on days 0 with aAPC, aAPCT or Vax-T, or immunized on day 0 and 14 with control vaccine formulations without (Soluble) or with (Soluble-T) TWS119. Endpoint titers were determined by ELISA for immunoglobulin M (IgM) on day 7 (FIG. 40B), IgG on week 4 (FIG. 40C). FIG. 40D: Endpoint titer ratios determined by ELISA for Th2-biased humoral responses (IgG1) and Th1-biased humoral responses (IgG2b) on week 4. FIG. 40E: Humoral responses specific to Spike protein at week 4 were assessed in serum from immunized animals by pseudovirus neutralization assay. FIG. 40F: SARS-CoV-2 surrogate neutralization endpoint titers at week 4. FIG. 40G: Kinetic of IgG titers. FIG. 40H: titer ratios of Th2-biased humoral responses (IgG1) and Th1-biased humoral responses (IgG2b). FIG. 40I: pseudovirus neutralization titers. FIG. 40J: SARS-CoV-2 surrogate neutralization titers as a function of time for vaccinated young and aged mice. Sham: Saline injection; Soluble: SARS-CoV-2 peptides/IL-2/CpG; Soluble-T: Soluble with TWS119. aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 PLGA nanoparticles; Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=5). The results were analyzed by using one-way ANOVA with post-hoc analysis. Statistical significance is indicated by the calculated p-values.

FIGS. 41A-41H show vaccination with Vax-T promoted potent T cell specific responses in healthy and immunocompromised mice against SARS-CoV-2. FIG. 41A: To create immunocompromised mice, mice received stem cell transplants two weeks prior to vaccination followed by daily injection of Cyclosporine A. Mice were immunized on day 0 with Vax-T and on day 0 and 14 with soluble (control) vaccine formulations. Humoral responses specific to Spike were assessed in serum from immunized animals after 1, 4, and 8 weeks. Eight weeks post-vaccination, splenocytes were isolated/stimulated to assess T cell responses. FIG. 41B: Micro-CT images of the ectopic bone formation 10 weeks after stem cell transplantation All specimens were normalized, and micro-CT images were calibrated to enable quantitative comparisons. FIG. 41C: Quantitative analysis of generated bone 10 weeks after stem cell transplantation. Relative volumetric bone formed as well as its average density were calculated. FIGS. 41D-41F: Humoral responses specific to Spike peptides were assessed in serum from immunized animals with immunosuppression by ELISA assay. Shown are immunoglobulin M (IgM) on day 7 (FIG. 41D), and IgG at week 4 (FIG. 41E) and IgG at week 8 (FIG. 41F) for vaccinated mice. Humoral responses specific to Spike were also assessed in serum from immunized animals by pseudovirus neutralization assay (FIGS. 41G-41H). Vaccination with Vax-T elicits potent SARS-CoV-2-specific CD8⁺ T cells in spleen. Eight weeks post-vaccination, splenocytes were stimulated with overlapping Spike peptides for 6 hours, and the response assayed by flow cytometry analysis (FIG. 41G) to detect antigen-specific T cell responses. Shown are frequencies of IFN-γ, TNFα, IL-2, and triple-positive T cells among CD8⁺ T cells. FIG. 41H: ELISA of cytokine production by spleen cells. n=5 mice per group. Vax-T: Alginate-heparin microparticles loaded with: CpG (5 nmol)+SARS-CoV-2 peptides (10 μg)+PLGA/TWS119 nanoparticles; Soluble: CpG (5 nmol)+SARS-CoV-2 peptides (10 μg); Soluble+T: Soluble+TWS119.

FIG. 42 shows particles were crosslinked with various concentrations of Ca2+ and PEG hydrazide to tune their mechanical stiffness, as measured by atomic force microscopy (AFM).

FIG. 43 shows heparin-functionalized alginate microparticles were synthesized and optimized to encapsulate cytokines IL-2, IL-7, and IL-15 for prolonged release.

FIG. 44 shows gating strategy for the identification of human T memory stem cells (TMSCs) before and after co-cultured with engineered aAPCs. Naive-like T cells were defined as CD45RO⁻CCR7⁺ in humans. In these gated populations, TMSCs express CD95 and CXCR3, whereas naïve T cells are CD95⁻CXCR⁻. Here TMSCs were identified in both CD4+ and CD8+ human T cells. As shown here, treatment with aAPCTs, i.e., alginate-heparin microparticles conjugated with CD3/CD28 antibodies on surface and loaded with soluble components (IL2 (100 U), CpG (5 nmol), SARS-CoV-2 peptides (10 μg), TWS119 (5 μM), IL-7/IL-15 (50 ng)) can significantly increase the population of TMSCs. Numbers indicate the percentage of cells in the indicated gates. T cell subsets were defined as follows: TN cells: CD3⁺ CD8⁺ (or CD4⁺) CD45RO⁻ CCR7⁺ CXCR3⁻ CD95⁻ and TMSCs: CD3⁺ CD8⁺ (or CD4⁺) CD45RO⁻ CCR7⁺ CXCR3⁺ CD95⁺.

FIG. 45 shows alginate-heparin conjugate was used as the main matrix to form aAPCS. TWS119 or similar small molecule inhibitors, as listed, were encapsulated in PLGA nanoparticles and then mixed with Spike peptides, cytokines, and alginate-heparin to form aAPCs. Following the crosslinking, surface of microparticles will be functionalized with anti-CD3 and anti-CD28 antibodies. TMSCs induction in vitro is modulated by the properties of aAPCs. Flow cytometry analysis of primary human naive CD8+ T cells before and 4 days after stimulation with aAPCs with various formulations in the presence or absence of 5 μM TWS119 or similar small molecule inhibitors. It should be noted that these inhibitors were loaded in microparticles in aAPCs formulations. The presented data are expressed as mean±SD (n=5). The results were analyzed by using one-way ANOVA with post-hoc analysis. Statistical significance is indicated by the calculated p-values. significant: p<0.05, very significant: p<0.01 and extremely significant: p<0.001.

FIG. 46 shows vaccination with CpG containing formulations elicits superior humoral responses compared to poly(I:C) and aluminum hydroxide (Alum). Vaccination with soluble formulations containing CpG, poly(I:C), or Alum (as a benchmark clinical adjuvant) were tested in the presence or absence of SARS-CoV-2 Spike peptides. To study humoral response against SARS-CoV-2 in vivo, C57BL/6 mice were immunized on days 0 and 14 with vaccine formulations containing CpG, poly(I:C), or Alum. CpG: CpG (50 μg)/IL-2 (100 U)±SARS-CoV-2 peptides (10 μg). poly(I:C): poly(I:C) (50 μg)/IL-2 (100 U)±SARS-CoV-2 peptides (10 μg). Alum: Alum (100 μg)/IL-2 (100 U)±SARS-CoV-2 peptides (10 μg). The presented data are expressed as mean±SD (n=5).

FIG. 47 shows cumulative in vitro release of CpG from aAPCs over time at 37° C. under gentle shaking (n=4).

FIG. 48 shows change in the mass of vaccine-draining lymph nodes (dLNs) post-vaccination. Successful vaccination can result in the accumulation and expansion of antigen-specific immune cells in dLN close to vaccination site. The mass of dLNs, which is driven by the proliferation of stromal cells and lymphocytes, showed trends similar to dLN stiffness post-vaccination. The presented data are expressed as mean±SD (n=3). Sham: Saline injection; Soluble: SARS-CoV-2 peptides/IL-2/CpG; Soluble-T: Soluble with TWS-119; aAPC: alginate-heparin/SARS-CoV-2 peptides/IL-2/CpG microparticles (5 um, 20 kPa) with medium level of anti-CD3/anti-CD28 surface antibodies; aAPCT: aAPC with TWS-119 PLGA nanoparticles; IL-2: 100 U/injection; CpG 50 ug/injection; SARS-CoV-2 peptides 10 μg/peptide/injection. The results were analyzed by one-way ANOVA with post-hoc analysis. Statistical significance is indicated by the calculated p-values. Significant: p<0.05, very significant: p<0.01 and extremely significant: p<0.001.

FIG. 49 shows injection site 3 days after vaccination.

FIG. 50 shows population of leukocytes (CD45⁺), T cells (CD3⁺), B cells (B220⁺), macrophages (F4/80⁺), dendritic cells (CD11c⁺MHCII⁺), CD11b⁺MHCII⁻, as well as CD4⁺ and CD8⁺ were assessed in vaccine-dLNs at 7 days post-vaccination. Sham: Saline injection; Soluble: SARS-CoV-2 peptides/IL-2/CpG; Vax-T: aAPCT without anti-CD3/anti-CD28 surface antibodies. IL-2: 100 U/injection; CpG: 50 μg/injection; SARS-CoV-2 peptides: 10 μg/injection. (n=3) Statistical significance is indicated by the calculated p-values. Significant: p<0.05, very significant: p<0.01 and extremely significant: p<0.001.

FIG. 51 shows biomaterial-based vaccine can induce stiffening of vaccine-dLNs in vivo in aged mice. Ten to thirteen-month-old C57BL/6 mice were immunized on days 0 with indicated formulations. Stiffening of vaccine-dLNs were monitored over time as a macroscopic evidence of vaccination efficacy. Area under the curve (AUC) was calculated for animal treated with various formulations. Sham: Saline injection; Soluble: SARS-CoV-2 peptides/IL-2/CpG; Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=3). The results were analyzed by one-way ANOVA with post-hoc analysis. Statistical significance is indicated by the calculated p-values. Significant: p<0.05, very significant: p<0.01 and extremely significant: p<0.001.

FIG. 52 shows biomaterial vaccine can induce the formation of TMSCs in vivo in aged mice. To study the formation of TMSCs in vivo, 10-13-month-old C57BL/6 mice were immunized on days 0 with Vax or Vax-T, or on day 0 and 14 with control vaccine formulations without (Soluble) or with (Soluble-T) TWS119. On day 21, vaccine-dLNs were extracted and population of TMSCs (CD8+ CD62L+ CD44− Sca-1+ T cells) were identified using flow cytometry. Isolated lymph node cells were sorted for naïve-like CD8+ T cell population (CD62L+ CD44−) and restimulated with overlapping peptide pool for 3 days, and the secretion of IFN-γ and IL-2 were quantified. Sham: Saline injection; Soluble: SARS-CoV-2 peptides/IL-2/CpG; Soluble-T: Soluble with TWS119; aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 PLGA nanoparticles; Vax: aAPC without anti-CD3/anti-CD28 surface antibodies; Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=5). The results were analyzed by one-way ANOVA with post-hoc analysis. Statistical significance is indicated by the calculated p-values. Significant: p<0.05, very significant: p<0.01 and extremely significant: p<0.001.

FIG. 53 shows polyfunctional recall CD4+ T cell responses improved upon sustained delivery by aAPCs but did not apparently require TWS119. To study the formation and function of SARS-CoV-2 specific CD4+ T cell response in vivo, young and aged C57BL/6 mice were immunized on days 0 with aAPC, aAPCT or Vax-T, or immunized on day 0 and 14 with control vaccine formulations without (Soluble) or with (Soluble-T) TWS119. Eight weeks post vaccination, spleen cells were stimulated with overlapping Spike peptides and assayed by flow cytometry for intracellular cytokines production to detect antigen-specific CD4+ T cell responses. Shown are frequencies of IFN-γ, TNF-α, IL-2 and triple-positive T cells among CD4+ T cells. Sham: Saline injection; Soluble: SARS-CoV-2 peptides/IL-2/CpG; Soluble-T: Soluble with TWS119; aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 nanoparticles; Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=5). The results were analyzed by one-way ANOVA with post-hoc analysis. Statistical significance is indicated by the calculated p-values. Significant: p<0.05, very significant: p<0.01 and extremely significant: p<0.001.

FIG. 54 shows the ratio of IFN-γ to IL-4 was significantly higher for aAPCT and Vax-T compared to the control soluble formulations in both young adult and aged population. To study formation and function of SARS-CoV-2 specific T cell response in vivo, young and aged C57BL/6 mice were immunized on days 0 with aAPC, aAPCT or Vax-T, or immunized on day 0 and 14 with control vaccine formulations without (Soluble) or with (Soluble-T) TWS119. Eight weeks post vaccination, spleen cells were isolated and stimulated with overlapping Spike peptides and assayed by ELISA for cytokines production to detect antigen-specific T cell responses. Ratio of secreted pro-inflammation cytokine IFN-γ and anti-inflammation cytokine IL-4 from splenocyte of vaccinated mice after being restimulated with overlapping Spike peptides was calculated. Sham: Saline injection; Soluble: SARS-CoV-2 peptides/IL-2/CpG; Soluble-T: Soluble with TWS119; aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 nanoparticles; Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=5). The results were analyzed by one-way ANOVA with post-hoc analysis. Statistical significance is indicated by the calculated p-values. Significant: p<0.05, very significant: p<0.01 and extremely significant: p<0.001.

FIG. 55 shows humoral responses against SARS-CoV-2 in young and aged mice. To study humoral responses against SARS-CoV-2, young (black bars) and aged (grey bars) C57BL/6 mice were immunized on days 0 with aAPC, aAPCT or Vax-T, or immunized on day 0 and 14 with control vaccine formulations without (Soluble) or with (Soluble-T) TWS119. Endpoint titers were determined by ELISA for immunoglobulin M (IgM) on day 7. Sham: Saline injection; Soluble: SARS-CoV-2 peptides/IL-2/CpG; Soluble-T: Soluble with TWS119; aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 PLGA nanoparticles; Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=5).

FIG. 56 shows humoral responses against SARS-CoV-2. To study humoral responses against SARS-CoV-2, C57BL/6 mice were immunized on days 0 with aAPC, aAPCT or Vax-T, or immunized on day 0 and 14 with control vaccine formulations without (Soluble) or with (Soluble-T) TWS119. Endpoint titer ratios were determined by ELISA for Th2-biased humoral responses (IgG1) on week 4 and 8. Sham: Saline injection; Soluble: SARS-CoV-2 peptides/IL-2/CpG; Soluble-T: Soluble with TWS119; aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 PLGA nanoparticles; Vax-T: aAPCT without anti-CD3/anti-CD28 surface antibodies. IL-2: 100 U/injection; CpG: 50 μg/injection; SARS-CoV-2 peptides: 10 μg/injection. Young (black bars), aged (grey bars). The presented data are expressed as mean±SD (n=5).

FIG. 57 shows humoral responses against SARS-CoV-2. To study humoral responses against SARS-CoV-2, C57BL/6 mice were immunized on days 0 with aAPC, aAPCT or Vax-T, or immunized on day 0 and 14 with control vaccine formulations without (Soluble) or with (Soluble-T) TWS119. Endpoint titer ratios were determined by ELISA for Th1-biased humoral responses (IgG2b) on week 4 and 8. Sham: Saline injection; Soluble: SARS-CoV-2 peptides/IL-2/CpG; Soluble-T: Soluble with TWS119; aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 PLGA nanoparticles; Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=5).

FIG. 58 shows biomaterial vaccine can induce stiffening of lymph nodes in vivo in immunocompromised mice. To create immunocompromised mice, mice received stem cell transplants two weeks prior to vaccination, followed by daily injection of Cyclosporine A. Immunocompromised mice were immunized on days 0 with indicated formulations. Stiffening of vaccine-dLNs was monitored over time as a macroscopic evidence of vaccination efficacy. Area under the curve (AUC) was calculated for animal treated with various formulations. Sham: Saline injection; Soluble: SARS-CoV-2 peptides/IL-2/CpG; Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=3). The results were analyzed by one-way ANOVA with post-hoc analysis. Statistical significance is indicated by the calculated p-values. Significant: p<0.05, very significant: p<0.01 and extremely significant: p<0.001.

FIG. 59 shows biomaterial vaccine can induce the formation of TMSCs in vivo in immunocompromised mice. To create immunocompromised mice, mice received stem cell transplants two weeks prior to vaccination followed by daily injection of Cyclosporine A. To study the formation of TMSCs in vivo, immunocompromised C57BL/6 mice were immunized on days 0 with Vax or Vax-T, or immunized on day 0 and 14 with control vaccine formulations without (Soluble) or with (Soluble-T) TWS119. On day 21, vaccine-dLNs were extracted and the population of TMSCs (CD8+CD62L+CD44⁻Sca-1+ T cells) were identified using flow cytometry. Isolated lymph node cells were sorted for naïve-like CD8+ T cell population (CD62L+CD44⁻) and restimulated with overlapping peptide pool for 3 days and secretion of IFN-γ and IL-2 were quantified. Sham: Saline injection; Soluble: SARS-CoV-2 peptides/IL-2/CpG; Soluble-T: Soluble with TWS119; aAPC: alginate-heparin microparticles (5 μm, 20 kPa) with medium level of anti-CD3/anti-CD28 surface antibodies, releasing SARS-CoV-2 peptides/IL-2/CpG; aAPCT: aAPC with TWS119 PLGA nanoparticles; Vax: aAPC without anti-CD3/anti-CD28 surface antibodies. Vax-T: aAPCT without anti-CD3/anti-CD28 surface antibodies. IL-2: 100 U/injection; CpG: 50 μg/injection; SARS-CoV-2 peptides: 10 μg/injection. The presented data are expressed as mean±SD (n=5). The results were analyzed by one-way ANOVA with post-hoc analysis. Statistical significance is indicated by the calculated p-values. Significant: p<0.05, very significant: p<0.01 and extremely significant: p<0.001.

FIG. 60 shows vaccination with Vax-T elicits potent humoral responses against SARS-CoV-2 in healthy and immunocompromised mice (week 1 and 4). Healthy mice and those with stem cell transplant receiving daily dose of Cyclosporine A were immunized on days 0 with Vax-T and on day 0 and 14 with soluble (control) vaccine formulations. Humoral responses specific to Spike peptides were assessed in serum from immunized animals by ELISA assay. Shown are immunoglobulin M (IgM) on day 7, and IgG on day 28 for vaccinated mice. Humoral responses specific to Spike peptides were also assessed in serum from immunized animals by pseudo virus neutralization assay. n=5 mice per group. Vax-T: Alginate microparticles loaded with: CpG (5 nmol)+SARS-CoV-2 peptides (10 μg)+PLGA/TWS119 nanoparticles; Soluble: CpG (5 nmol)+SARS-CoV-2 peptides (10 μg); Soluble+T: Soluble+TWS119.

FIG. 61 shows hematological biosafety evaluation of Vax-T. Hematology for naïve mice treated with either PBS injections (Control) or with Vax-T formulation was examined 7 days post-vaccination.

FIG. 62 shows blood biosafety evaluation of Vax-T. Blood chemistry for naïve mice treated with either PBS injections (Control) or with Vax-T formulation was determined 7 days post-vaccination.

FIG. 63 presents tissue biosafety evaluation of Vax-T, showing histology analysis of major organs (heart, liver, spleen, lung, and kidney) of mice 7 days post-vaccination with Vax-T formulation. Images are presented in two magnifications: 10× and 40×.

FIG. 64 shows cancer vaccination with Vax-CT promoted potent anti-tumoral T cell specific responses in mice against B16-F10 melanoma. Sham: Saline injection; Soluble: B16-F10 melanoma tumor lysate/IL-2/CpG; Vax-C: PLGA/B16-F10 melanoma lysate/IL-2/CpG microparticles (10 um); Vax-CT: Vax-T with TWS-119. IL-2: 100 U/injection; CpG 50 ug/injection; melanoma lysate 100 μg/injection. The presented data are expressed as mean±SD (n=5). The results were analyzed by using one-way ANOVA with post-hoc analysis. Statistical significance is indicated by the calculated p-values. Significant: p<0.05, very significant: p<0.01 and extremely significant: p<0.001.

FIG. 65 shows designed artificial antigen presenting cells can outperform dynabead and provide more powerful tumor fighting T cells. aAPCT: alginate-heparin/IL-2 microparticles (5 um, 20 kPa) with medium level of anti-CD3/anti-CD28 surface antibodies with TWS-119 PLGA nanoparticles; IL-2: 100 U/injection. The presented data are expressed as mean±SD (n=5). The results were analyzed by using one-way ANOVA with post-hoc analysis. Statistical significance is indicated by the calculated p-values. Significant: p<0.05, very significant: p<0.01 and extremely significant: p<0.001.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the disclosure.

In the following detailed description, numerous specific details are set forth in order to provide a thorough understanding of implantable scaffolds and microparticles, and the uses thereof. However, it will be understood by those skilled in the art that the production of these implantable scaffolds and microparticles and uses thereof may be practiced without these specific details. In other instances, well-known methods, procedures, and components have not been described in detail so as not to obscure their description.

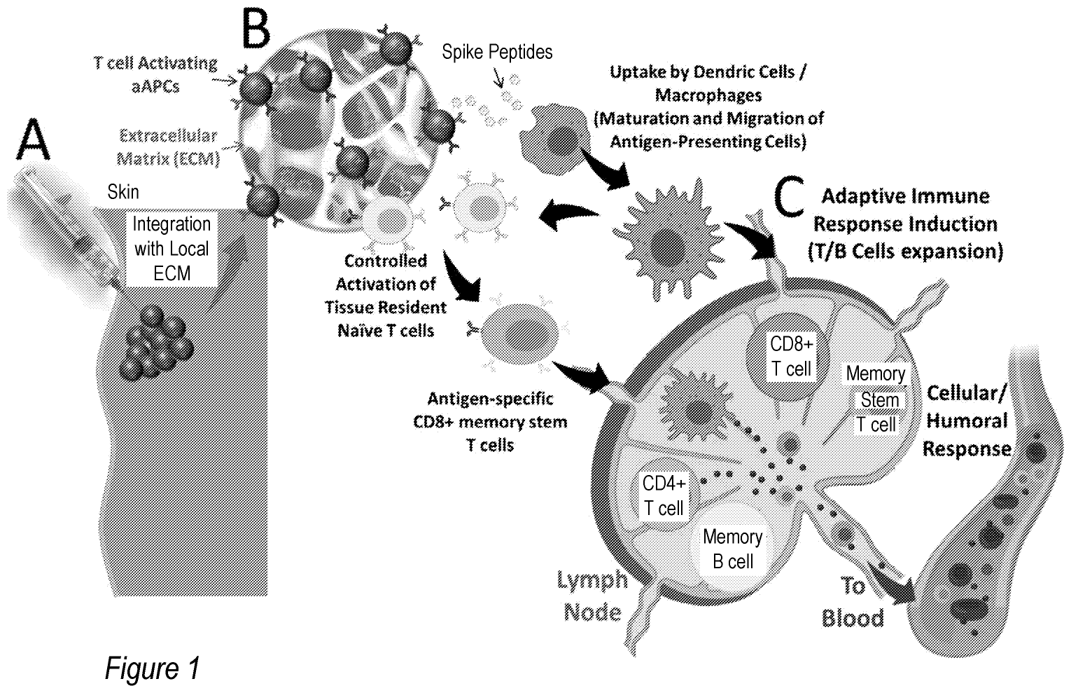

Disclosed herein is the development of, in one embodiment, a series of injectable cell-free, biomaterial-based vaccine boosters with tunable mechanical and biophysical properties which present polyclonal or antigen-specific signals that can engage with tissue resident immune cells and enhance long-term immunity. These microparticle platform was designed to be, among other non-limiting properties, to be (i) injectable (ii) biodegradable, (iii) engage with naïve T cells using CD3/CD28 antibodies, (iv) release cytokines to enhance the activation and expansion of T cells, (v) release regulators to induce the formation of TMSCs, and (vi) co-deliver a vaccine candidate. In one example, the vaccine candidate is a peptide pool created based on the Spike protein of SARS-CoV-2 (FIG. 1a). This platform is low cost compared to cell-based vaccines and can be readily scaled up for commercial production in GMP facilities to provide affordable vaccines against endemic and epidemic pathogens such as but not limited to SARS-CoV-2 to address global health issues.

As will be described below and shown in the Examples, two variations of the disclosed microparticle formulations, aAPCT and Vax-T, induce robust and prolonged Th1 T cell responses. Given the need for immunization against SARS-CoV-2 and similar pathogens on a global scale, Vax-T as the optimized vaccine candidate for such uses. Vax-T with its simplified design, can not only trigger similar humoral and cellular immune responses compared to aAPCT, but also outperformed in other properties. Vax-T can provide a very strong and prolonged immunity just after a single dose administration not only in adult and aged mice but also in the mice suffering from immunosuppression. While the exemplary formulation employed RBD peptides as model antigen candidates, mRNA-based or attenuated viruses reagents can also be integrated in the platform disclosed herein with some modification to be served as alternative vaccines.

Blood and tissue toxicology of the exemplary Vax-T formulation one week post-vaccination showed no significant change in the standard hematology markers including white blood cells count, red blood cells count, hemoglobin, hematocrit, mean corpuscular volume, mean corpuscular hemoglobin, mean corpuscular hemoglobin concentration, and platelet count. Major organs, including heart, liver, spleen, lung, and kidney were also examined for the possible histological alterations. No detectable abnormalities or lesions in organs were identified, thus demonstrating lack of safety concerns of the microparticle formulations disclosed herein.

The present disclosure is directed to boosting T memory stem cells (TMSCs), in particular but not limited to the elderly, to elicit a robust immune response. In one embodiment, TMSCs are unique subsets of memory lymphocytes endowed with the stem cell-like ability to self-renew and the multipotent capacity to reconstitute the entire spectrum of memory and effector T cells. Maintenance of long-lived T cell is required because increased T cell loss after the peak period is a main reason of diminishing memory in the elderly.

In one embodiment, an injectable, cell-free, and biomaterial-based microparticles composition is disclosed that can engage with tissue resident immune cells and enhance long-term immunity. This platform is low cost compared to cell-based vaccines, can be scaled up following Good Manufacturing Practice (GMP) to provide affordable vaccine booster against endemic and epidemic pathogens such as but not limited to SARS-CoV-2. Such TMSC booster can be translated into clinical applications, and co-delivered with vaccines for SARS-CoV-2, flu viruses and many other pandemic pathogens, which will in one embodiment stimulate long-term immunity, and in one embodiment offer protection for the elderly populations and those with declined immune responses to vaccines, and greatly benefit healthcare.

In one embodiment, immunomodulatory injectable microparticles comprising a 3D scaffold are disclosed that mimic the functions of artificial antigen presenting cells (aAPC) and induce TMSCs against SARS-CoV-2. As will be described in the example herein, a peptide pool created based on the Spike protein of SARS-CoV-2 is used as an exemplary potential vaccine component. As will be described herein, the injectable 3D scaffold has tunable physicochemical properties, and in some embodiments achieves multiple functional aspects, such as but not limited to: (i) recruit tissue resident T cells, (ii) provide cell- and extracellular matrix (ECM)-mimicking environment for cellular engagement, (iii) provide optimal activation signals to activate naïve T cells, (iv) release cytokines and small molecules to induce the formation of TMSCs, (v) co-deliver a vaccine candidate, in one example, a peptide pool created based on the Spike protein of SARS-CoV-2. Thus, the 3D scaffold is cell-free, easy-to-handle, injectable, biodegradable, and scalable platform with modular antigen/cytokine/adjuvant loading/release properties, which will have wide applications in immunomodulation for many diseases. In other embodiments, any one of more of the aforementioned components or features may be excluded without deviating with the objectives of the microparticles disclosed herein. Furthermore, any one or more components or features may be included in the microparticles to support or enhance the aforementioned activities.

As disclosed herein, in the non-limiting example of a composition to enhance formation of SARS-CoV-2 antigen presenting TMSCs, the microparticle compositions act as temporary artificial lymph nodes to present viral antigens as well as providing sustained release of required cytokines while providing the optimized activation signals to promote formation of TMSCs. Such injectable biomaterial-based microgels, which specifically interact with primary T cells while provide optimized activation signals and present required cytokines (e.g., IL-2, IL-7), can generate mature antigen-specific TMSCs. To prepare the composition, in one non-limiting embodiment, a microfluidic device featuring a droplet generator may be used to form microparticles. Alginate-based microparticles comprising various concentrations of polymer from 0.5 to 5 wt % are prepared, and in some embodiments microparticles are crosslinked to increase mechanical stiffness. In some embodiments the microparticles comprise heparin. Alternate biocompatible and biodegradable polymers may also be used in place of on in combination with alginate to provide microparticles with the features disclosed herein.

As shown in the examples below, to demonstrate proof of concept, recombinant T cell receptor (TCR) and CD4 molecules are conjugated to the microparticles, and quantitative flow cytometry used to estimate and optimize TCR and CD4 conjugation density of proteins on the surface of microparticles, to achieve the values consistent with natural cells (see, for example, Hasani-Sadrabadi M M, Majedi F S, Bensinger S J, et al. Mechanobiological Mimicry of Helper T Lymphocytes to Evaluate Cell-Biomaterials Crosstalk. Advanced Materials 2018; 30: 1706780). As shown in the examples herein, microparticles of tunable size and mechanics are prepared, which can be decorated with a wide range of different surface proteins or targeting antibodies, depending on their intended applications. In one example, stimulatory antibodies (e.g., αCD3; TCR stimulus and αCD28; costimulatory cue) were conjugated alginate-heparin-based microparticles that emulate an artificial APC in terms of size and mechanical stiffness. Experiments here were titrated the range of antibody coating. Alginate and calcium concentrations are manipulated during microparticle formation to optimize properties. In one non-limiting embodiment, alginate in the range of 0.5 to 2.5 wt % and Ca²⁺ in the range of 10-60 mM during formation allows for sampling a mechanical space of roughly 1-150 kPa, i.e., the entire physiological range of mechanical stiffness. In some embodiments, a stiffness of 2-60 kPa is provided. The impact of altering antibodies and the mechanical stiffness are evaluated on the proliferation, activation markers, and cytokine production by co-culture with naïve mouse and human T cells while providing soluble cytokines required for formation of T memory stem cells and following the cell surface markers. Optimized microparticles are thus identified with the properties of and for the purposes as disclosed herein.

As described herein, the properties of the microparticles are evaluated using a number of tests to identify and optimize their desired properties. In one embodiment, the kinetics of cytokine (e.g., IL-2, IL-7, IL-15) uptake and release from microparticles to tune T cell differentiation is determined. For example, heparin-functionalized alginate microparticles (5-15 μm in diameter) are synthesized and optimized to encapsulate target cytokines IL-2, IL-7, and IL-15. Heparin-based conjugates are developed at several conjugation densities. Carbodiimide chemistry (e.g., NHS/EDC) are utilized to modify amine-modified alginate. Heparin, an optional component of the microparticles disclosed herein, provides enhanced efficiency and stability of cytokine that enables precise spatiotemporal control over the release profile of target cytokines. As shown below, the heparin-modified particles improved the binding and prolonged release of IL-2 compared to alginate particles alone.

In addition, in some embodiments, microparticles disclosed herein comprise an agent that enriches TMSC formation. In some embodiments, such enrichment is provided by small molecule TWS119 (3-[[6-(3-Aminophenyl)-7H-pyrrolo[2,3-d]pyrimidin-4-yl]oxyphenol ditrifluoroacetate), a potent inhibitor of the serine-threonine glycogen synthase kinase 3β (GSK-3β), is able to induce the Wnt-β-catenin signaling and the enrichment of TMSC through differentiation inhibition both in mice and in human. TWS119 may be included in the microparticles described herein for TMSC induction. TWS119 is hydrophobic and can be loaded into poly(lactic-co-glycolic acid) (PLGA) nanoparticles for a sustained release. The kinetics of TWS119 release from aAPCs with various initial loading (1, 5, 10, and 20 μM) is evaluated in vitro using HPLC. In some embodiments, target delivery regimes are reached for each dose by modifying the formulation and composition. In other embodiments, an alternate TMSC enriching compound or agent is included in the microparticles disclosed herein.

Selection of the antigen or immunogen is dependent on the desired immune response to be enhanced by the microparticles disclosed herein. In some embodiments, to facilitate peptide antigen development, a SARS-CoV-2 Spike Glycoprotein peptide pool is employed, such as that from GenScript (Piscataway, NJ). This pool includes 316 peptides derived from a peptide scan (15mers with 11 amino acid overlap) through the entire Spike glycoprotein (Protein ID: P0DTC2) of SARS-CoV-2. Various concentration of these peptide mixture are loaded in the microparticles during particle formation. Release of peptides is assessed by micro BCA protein assay. In other embodiments, antigens to elicit an immune response against, by way of non-limiting examples, infectious agents or cancers.

In some embodiments, APCs have an important role in the activation of other immune cells is by making active engagement as well as supplying them with cytokines known to increase activation/expansion and encourage the formation of pathogen reactive T cells. In some embodiments, optimizing the size and surface receptor densities to alter the activation and proliferation is performed.

Non-limiting examples of methods are used to prepare and evaluate the microparticles disclosed herein. In some embodiments, young and old wild type C57/B6 mice (Jackson Labs) are used for murine in vitro experiments. Cell-culture media is RPMI supplemented with 10% heat-inactivated FBS, 1% penicillin/streptomycin, 1% sodium pyruvate, 1% HEPES buffer, 0.1% μM 2-mercaptoethanol. CD4+/CD8+ T cells are purified from mice spleens and lymph nodes using negative selection enrichment kits (Stem Cell Technologies). Peripheral blood mononuclear cells (PBMCs) from healthy human donors are used as cell source for naïve (progenitor) human T cell source in this study to generate human T memory stem cells in vitro. PBMCs are isolated by Ficoll-Hypaque gradient separation (Lymphoprep, Fresenius) and enriched for CD3+ cells with the human CD3 MicroBeads Isolation Kit (Miltenyi Biotec) following the manufacturer's instruction. Costar transwell inserts (pore size 1 μm) are used for assessing the effectiveness of the released factors on the activation, maturation, and differentiation of primary naïve T cells. Microgels containing cytokines were placed in the upper chamber. The bottom chamber contains 10⁵ naïve T cells. After 48 h of stimulation, the activation markers are tested by flow cytometry analysis, and cells are cultured for 5, 7, and 14 additional days in order to evaluate the ex vivo expansion and assess the sternness of treated cells. The change in T cell activation, differentiation, and formation of TMSCs is tested at using flow cytometry by checking the surface markers including CD4, CD8, CD62L⁺, CD45RO⁻, CD45RA⁺, CD27⁺, CD95⁺, CD122⁺, and CCR7⁺ for human T cells and CD4, CD8, CD62L⁺, CD44⁻, CD122⁺, and Sca-1⁺ for murine T cells while expressing intracellular IFN-γ cytokine and not IL-4. For intracellular cytokine staining (ICCS), cells are fixed with cold 4% paraformaldehyde (PFA) for 20 min at room temperature and then permeabilized with 0.5% saponin for 10 min before blocking with PBS+1% BSA+5% donkey serum and staining with appropriate antibodies at 1:100 dilution. Cells are washed and analyzed by flow cytometry as above. The following antibodies were used for ICCS from Biolegend: IL-2, IL-4, IL-10, IL-17A, IFN-γ, and TNF-α. The cells are assessed for T cells skewing using intracellular cytokine staining (ICCS) for IFN-γ and IL-4. Th1 cells should show more IFN-γ production than IL-4 (which is the major cytokine of Th2 cells). The optimal levels of stiffness/CD3/CD28/IL-2 combination for T cell activation are then used for further investigations.

In vivo studies are carried out to evaluate the microparticles disclosed herein. While not wishing to be bound by theory, injectable immunomodulatory microparticles or scaffold can interact with naïve T cells while providing optimized activation, maturation, and differentiation signals, and thus offer a significant therapeutic advantage for immunization, especially in aged animals. In some embodiments, developed biomaterial vaccine booster was injected subcutaneously. Cellular responses were analyzed in TMSC induction and humoral response via neutralizing antibody responses generated against Spike glycoprotein of SARS-CoV-2. The effects of animal age and sex are investigated on TMSC formation and neutralizing antibody production by using murine model such as reported before for SARS-CoV. Young (6-10 weeks-old) as well as old (10-13 month-old) mice are used.

In some embodiments, in order to analysis SARS-CoV-2-specific activated T cell, vaccine site draining lymph node(s), lung, bone marrow, and spleen are removed from mice at several time points (e.g., 7, 14, 28, and 56 days) after immunization with immunomodulatory microparticles/scaffolds and control groups, and single cell suspensions are prepared in RPMI 1640 containing 10% fetal calf serum (FCS). Following washing, cells are analyzed by flow cytometry to study T cell activation and differentiation as well as analyzing TMSC population based on expression of surface markers as well as intracellular cytokine secretion in T cell subset. The effects of age, sex, T cell recruitment chemokine, TMSC induction signals and Spike protein antigens on the induction of TMSCs are thus compared.

As shown in the examples below, humoral response were demonstrated via neutralizing antibody generated against Spike glycoprotein peptides of SARS-CoV-2. Mice were bled from the saphenous vein at week 0, 1, 2, 4, and 8, and sacrifice by the end of week 8. Serum samples are evaluated by ELISA for SARS-CoV-2-S1 antibody production. Anti-SARS-CoV-2 IgG and IgM ELISA are performed using beta-versions of commercial kits according to manufacturers' protocols.

Microparticles. In certain embodiments, disclosed herein are microparticles. In some embodiments, the microparticles comprise a polymer. In some embodiments, the polymer comprises a biocompatible polymer. In some embodiments, the polymer comprises a biodegradable polymer. In some embodiments, the biocompatible polymer comprises alginate, chitosan, or mesoporous silica. In some embodiments, the microparticle has a size of about 0.1 μm to about 1000 μm. In some embodiments, the microparticles are about 0.4 to about 50 μm in diameter, and in some embodiments, about 3 to about 24 μm in diameter. In some embodiments the microparticles are about 200 to about 800 μm in diameter, and the average pore size of the microparticles are about 5 μm to about 100 μm. In some embodiments, the pores are about 5 μm to about 15 μm. In some embodiments, the pores are about 20 μm to about 100 μm. In some embodiments, the stiffness of the microparticle is about 1 to about 150 kPa. In some embodiments, the stiffness is about 2 to about 60 kPa.

In some embodiments, the microparticles comprise hyaluronic acid. In some embodiments, the microparticles comprise heparin.

In some embodiments, microparticles may be encapsulated by a coating. In some embodiments, coatings provide microparticles with enhanced biological characteristics, including interactions with cells, with compounds that regulate T cell immune response, with compounds that regulate induction of regulatory T cells, and with other biomolecules. In some embodiments, microparticles are encapsulated with a coating comprising heparin. In some embodiments, microparticles are encapsulated with alginate or alginate-heparin. In some embodiments, an alginate-heparin coating may be sulfated.

In some embodiments, microparticles may comprise a “coating” material. In some embodiments, these materials provide microparticles with enhanced biological characteristics, including interactions with cells and biomolecules. In some embodiments, microparticles are formed in the presence of a mix of alginate-heparin. In some embodiments, microparticles are formed in the presence of a mix of alginate. In some embodiments, an alginate may be sulfated.

A skilled artisan would appreciate that a description of a microparticle comprising an alginate or alginate-heparin coating may in certain embodiments, encompass a microparticle prepared in the presence of alginate or alginate and heparin, wherein these molecules and integral components of the microparticle synthesized.

In some embodiments, microparticles may be targeted to T cells. In some embodiments, a microparticle coat comprises biomolecules that recognize and bind cell surface markers on T cells. In some embodiments, cell surface markers on T cells include CD3 and CD28. In some embodiments, a biomolecule that recognized a T cell surface marker comprises an antibody or a fragment thereof.

Paramagnetic nanoparticles may be included in the microparticles, e.g., for purification or for ease of separation, and are commercially available (e.g., CHEMICELL™ GmbH). In some embodiments, the paramagnetic nanoparticles comprise superparamagnetic iron oxide nanoparticles (SPIONs). In some embodiments, a SPION comprises a particle having a size about 50-200 mm. This addition may in certain embodiments enhance purification of microparticles using methods well known in the art.

In some embodiments, described throughout herein, are “porous scaffolds.” These scaffolds are able to provide T cell immunoregulatory compounds to a microenvironment within which they are implanted and located. The release of immunoregulatory compounds may be regulatable, providing targeted therapeutic biological molecule(s) or biological molecule(s) that in turn regulates a downstream therapeutic target. This regulatable production may in certain embodiments, reduce or eliminate systemic toxicity.

In some embodiments, described throughout herein, are “microparticles.” These microparticles are embedded in a scaffold. These microparticles may serve as a “platform” comprising at least one compound that enhances the T cell immune response, providing an increased amount of a biomolecule or other compound that regulates T cell immune response, while retaining the regulatable aspects of the localized distribution.

In some embodiments, the scaffold is biocompatible or biodegradable. In some embodiments, the scaffold comprises a polymer selected from alginate, hyaluronic acid and chitosan, or any combination thereof. In some embodiments, the polymer comprises an arginine-glycine-aspartate (RGD) peptide. In some embodiments, the porous scaffold comprises pores of average pore size of about 20 μm to about 100 μm.

In some embodiments, the scaffold is provided to be surgically implantable or injectable or administrable through a catheter.

As will be apparent from the description herein, the microparticles disclosed herein may comprise multiple components or compartments therein. In one embodiment, the microparticles comprise a cross-linked polymer matrix entrapping nanoparticles, wherein nanoparticles may comprise a TMSC inducer or any other component descried herein. In one non-limiting example, nanoparticles are made of PLGA. In one non-limiting embodiment nanoparticles are 1 about 100 to about 300 nm, e.g., about 150 nm. Nanoparticles may comprise a single additional component, or they may comprise two or more components. For example, during preparation of the microparticles herein and before cross-linking of the polymer, PLGA nanoparticles comprising TWS119 (at, for example 0.1-20 μM). During preparation, in one example, 1-1000 parts of nanoparticles are included in 1-100 parts of polymer matrix.

Polymer matrix. In some embodiments, the polymer matric comprises alginate crosslinked with calcium. In some embodiments the concentration of alginate is 0.5% to about 5% w/v. In some embodiments the concentration of alginate is 2.5% w/v. In some embodiments the concentration of calcium is about 10 to about 60 mM.

As described above, the polymer matrix is formed into microparticles. In some embodiments, the particles are from about 0.5 to about 50 μm in diameter, and in some embodiments, about 3 to about 24 μm in diameter. In some embodiments the average pose size of the polymer matrix is about 65 μm. In some embodiments, the stiffness of the polymer matrix is about 1 to about 150 kPa. In some embodiments, the stiffness is about 2 to about 60 kPa.

In some embodiments, the polymer matrix comprises PLGA. In other embodiments, the polymer matrix comprises any biocompatible, biodegradable polymer capable of comprising and delivering the components described further below.

In some embodiments, the polymer matrix may be modified, e.g., by modifying carboxylic acids with NHS, for subsequent crosslinking to proteins (e.g., antibodies, RGD peptides) using, for example, EDC. Any non-covalent association or covalent association between one or more components of the microparticles disclosed herein and any other components of the microparticle is embraced herein.

In some embodiments, the porous scaffold comprises an alginate-RGD polymer comprising silica-heparin microparticles bound to IL-2, anti-CD3 and anti-CD28, PLGA nanoparticles comprising a TMAC inducer, and anti-CD3 and anti-CD28 antibodies covalently bound to the alginate-RGD polymer.

Antigen. The microparticles disclosed herein provide an antigen to which an enhanced immune response is elicited in the subject into which is administered, and in some embodiments, enhances or provides an immune response in individuals with immunosenescence, in particular, the elderly. However the compositions and methods disclosed herein are not applicable to any particular population but those in need of, or desirous of having, or upon the advice of a health care professional, of being effectively vaccinated against a particular antigen, virus or other microorganism, or even against a cancer. Thus, there is no limitation on the appropriate chronological age of a subject. A subject with a suboptimal immune system is, in one embodiment, a candidate for immunization with a composition disclosed herein.

In some embodiments, the microparticles are used to vaccinate a subject for preventative purposes, such as to prevent infection with a virus (e.g., COVID, influenza) or bacterium (e.g., pneumonia). In some embodiments, the microparticle are used to vaccinate a subject for therapeutic purposes, e.g., where the subject has cancer or an infectious disease.

In some embodiments, the subject is elderly, e.g., is over 60 years of age, over 65 years of age, over 70 years of age, over 75 years of age, over 80 years of age, over 85 years of age, or over 90 years of age. In some embodiments, the subject has immunosenescence. In some embodiments, the subject is immunocompromised. In some embodiments, the subject has an immunodeficiency disorder. In some embodiments, the subject is undergoing cancer therapy.

Studies described herein utilized as an example the immunogen of the coronavirus responsible for COVID19, i.e., SARS-CoV-19, and the spike protein thereof as an antigenic protein to demonstrate the effectiveness of the approach disclosed herein. Such protein is a non-limiting example of antigens or immunogens that may be incorporated into the microparticles disc disclosed herein. Other examples include hepatitis A, hepatitis B, hepatitis C, influenza, polio, rabies, measles, mumps, rubella, rotavirus, smallpox, chickenpox, yellow fever, Haemophilus influenzae type b disease, human papillomavirus, whooping cough, pneumonococcal disease, meningococcal disease, shingles, diphtheria, tetanus, by way of non-limiting examples. Vaccines for other infectious viral, bacterial, protozoan, helminthic, and other organisms is embraced herein. Furthermore, vaccines against cancers such as Wilms tumor antigen expressing cancers, are embraced herein, including but not limited to alphafetoprotein, carcinoembryonic antigen, CA-125 ovarian cancer, MUC-1 breast cancer, epithelial tumor antigen, tyrosinase, melanoma-associated antigen (MAGE) and abnormal products of ras, p53.

In some embodiments, the tumor antigen is specific for a tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome.

In one embodiment, the tumor is a solid tumor.

The antigen, in one embodiment, is incorporated into the polymer matrix of the microparticle, e.g., in the case of an alginate based polymer, incorporated into alginate before calcium cross-linking to form microparticles. Incorporation into other polymer matrices is embraced herein. In other embodiments, the antigen may be incorporated into a compartment within the microparticle, such as but not limited to in or on nanoparticles.

The amount of antigen incorporated into the microparticles is based on the dose or amount of microparticles administered to the subject. The amount may be guided by the amount of antigen used in conventional vaccines, and may be reduced by at least 10%, at least 20%, at least 30%, at least 40%, at least 50%, at least 60%, at least 70%, at least 80%, at least 90%, or at least 95%, of the conventional dose.

In some embodiments, the amount of antigen is provided to deliver a dose of antigen of from about 10 micrograms to about 10,000 micrograms per kilogram body weight, for example, to generate a humoral response. In some embodiments, the amount of antigen is about 10-1000, 10-1000, 10-500, 100-500, 100-1000, or about 100-10,000 micrograms per kilogram body weight.

In some embodiments, the amount of antigen is about 500 micrograms per kilogram of body weight.

In some embodiments, the microparticles disclosed herein are exposed to blood, blood components or T cells ex vivo before infusion of the blood, blood components or T cells into a patient.

T cell immunostimulatory component. In some embodiments, the microparticles disclosed herein comprise a T cell immunostimulatory compound. In some embodiments, the T cell immunostimulatory compound comprises interleukin-2 (IL-2), interleukin-4 (IL-4), interleukin-6 (IL-6), interleukin-7 (IL-7), interleukin-10 (IL-10), interleukin-12 (IL-12), interleukin-15 (IL-15), IL-2 superkine, chemokine (C-C motif) ligand 21 (CCL21), anti-cluster of differentiation 3 (anti-CD3), or anti-cluster of differentiation 28 (anti-CD28), or any combination thereof.

The T cell immunostimulatory component may be provided within the polymer matrix by admixture while the polymer is being cross-linked. In other embodiments, it may be incorporated into nanoparticles within the polymer matrix. In other embodiments it may be bound to heparin which is included in the polymer matrix.

The amount of T cell immunostimulatory component is provided to stimulate T cells. In one embodiment, the microparticles contain about 20 ng per mL of microparticle. In some embodiments, the T cell immunostimulatory component is IL-7 and in some embodiments is present at about 25 ng per mL of microparticle. In some embodiments, the T cell immunostimulatory component is IL-15 and in some embodiments is present at about 25 ng per mL of microparticle. In some embodiments two or more T cell immunostimulatory components are included, such any two from among IL-2, IL-7 and IL-15.

T memory stem cell (TMSC) inducer. In some embodiments, the porous scaffold further comprises a T memory stem cell inducer, such as but not limited to TWS119 (3-[[6-(3-Aminophenyl)-7H-pyrrolo[2,3-d]pyrimidin-4-yl]oxyphenol ditrifluoroacetate; CAS No. 601514-19-6). In some embodiments, the TMSC inducer is provided in nanoparticles within the polymer scaffold. In some embodiments, the TMSC inducer is provided in the polymer matrix. In some embodiments, the nanoparticles comprise poly(lactic-co-glycolic acid) (PLGA). In some embodiments, the TMSC inducer is TWS119 and is provided in PLGA nanoparticles that are incorporated into the microparticles when the polymer matrix is prepared.

In some embodiments, the scaffold comprises one or more nanoparticles. In some exemplary, but non-limiting, embodiments, the nanoparticle comprises a poly(lactic-co-glycolic acid) (PLGA, PLG), a copolymer, produced using methods known in the art. In some embodiments, the nanoparticle is sized between about 100 and about 300 nm. In some embodiments, nanoparticles are about 150 nm. In some embodiments, the nanoparticle is biocompatible and/or biodegradable. This addition may in certain embodiments enhance purification of microparticles or nanoparticles using methods well known in the art.

In some embodiments, the concentration of TMSC inducer, in the case of a small molecule such as but not limited to TWS119, is between about 1 and about 20 μM. Other TMSC inducers, such as but not limited to biological molecules, proteins, antibodies, etc. may be included alone or in combination with a small molecule inducer.

Adjuvant. The microparticles further comprise an adjuvant. Non-limiting examples of adjuvants include alum, Polyinosinic:polycytidylic acid (poly(I:C)), and CpG oligodeoxynucleotide (CpG). Poly (I:C) is known to interact with toll-like receptor 3 (TLR3), which is expressed at the endosomal membrane of B-cells, macrophages and dendritic cells. Poly I:C is structurally similar to double-stranded RNA, which is present in some viruses and is a “natural” stimulant of TLR3. Thus, Poly I:C can be considered a synthetic analog of double-stranded RNA and is a common tool for scientific research on the immune system. Its CAS Reg. No. is 24939-03-5. CpGs are synthetic oligodeoxynucleotides (ODNs) containing unmethylated CpG motifs trigger cells that express Toll-like receptor 9 (including human plasmacytoid dendritic cells and B cells) to mount an innate immune response characterized by the production of Th1 and proinflammatory cytokines. The CpG may be class A, class B, or class C. Non-limiting examples of CpG include ODN 1585, ODN 2216, ODN 2336, ODN 1668, ODN 1826, ODN 2006, ODN 2007, ODN BW006, ODN D-SL01, ODN 2395, ODN M362 or ODN D-SL03, available from Invivogen, San Diego CA.

The aforementioned components of the microparticles disclosed herein may comprise one or more additional components. Any combination of one or more additional components are embraced herein.

Cell adhesion/attraction components. Any one or more cell adhesion and/or cell attraction and/or immunostimulatory compounds or components may be included in or on the scaffold. In one embodiment, such components attract or activate T cells. Non-limiting examples include CCL21, anti-CD3 antibodies, anti-CD28 antibodies, or any combination thereof. In one embodiment, a combination of anti-CD3 and an anti-CD28 antibodies are used. Any one or more immunostimulatory components may be included in or on the scaffold. In some embodiments, CCL19, CCL17 or CCL22 are provided. In some embodiments, components such as but not limited to IL-2, IL-4, IL-6, IL-7, IL-10, IL-12 IL-15, or IL-2 superkine are used, singly or in any combination. In one embodiment as described below, such components may be bound to mesoporous silica microparticles and/or to heparin-modified mesoporous silica microparticles comprising the scaffold. In another embodiment, post-modification of the scaffold is performed to conjugate anti-CD3 and anti-CD28 antibodies through EDC/NHS cross-linking reagents, to provide stronger T cell activation signals. In other embodiments, such compounds or components suppress T cell attraction or T cell activation. Additional embodiments are described elsewhere herein.

T cell activators. Anti-CD3 antibodies and/or anti-CD28 antibodies may be provided in the microparticles in the polymer matrix, or may be formulated in nanoparticles incorporated into the microparticles during preparation.

Chemokines. CCL21, CCL19, CCL17 and/or CCL22 may be provided I the microparticles, in the polymer matrix, or may be formulated in nanoparticles incorporated into the microparticles during preparation.

Heparin. Heparin may be included in the polymer matrix as it provides a scaffold for the non-covalent binding of cytokines and other components. In some embodiments, the heparin is bound to silica in the microparticles. In some embodiments, about 2 nmol of heparin is bound per mg of silica.

Silica. Silica particles (mesoporous silica) may be included in the microparticles as a site for adsorption of cytokines and other components of the microparticles. In some embodiments, the silica is bound to heparin. In some embodiments, about 2 nmol of heparin is bound per mg of silica. In some embodiments, the microparticle has a size comprising 1-1000 micrometers.

Methacrylate. Methacrylate or another acrylic-based polymer may be included in the microparticles disclosed herein.

Other polymers useful for making the microparticles disclosed herein include polycaprolactone (PCL), poly(lactic acid) (PLA) and any other biocompatible polymer.

RGD Peptide. Peptides comprising the RGD sequence (L-Arginyl-Glycyl-L-Aspartic acid; Arg-Gly-Asp) for providing additional T cell attraction and/or T cell attachment. Non-limiting examples of peptides that may be used include RGDS, GRGD, GRGDS, GRGDSP, GRGDSPK, GRGDNP, GRGDTP, by way of non-limiting examples. The RGD peptide may be conjugated to the polymer matrix (e.g., alginate) using NHS/EDC cross-linking or any other means.

In some embodiments, the nanoparticle is bound to at least one compound that regulates induction of regulatory T cells, as described herein.

In some embodiments, the silica is bound to heparin. In some embodiments, about 2 nmol of heparin is bound per mg of silica. In some embodiments, the microparticle has a size comprising 1-1000 micrometers.

Non-limiting examples of microparticle compositions are described below.

In some embodiments, the microparticles disclosed herein comprise at least one immunogen (such as a COVID spike protein), and at least one T memory stem cell (TMSC) inducer (such as TWS119).

In some embodiments, the microparticles disclosed herein comprise at least one immunogen (such as a COVID spike protein), at least one immunostimulatory cytokine (such as IL-2, IL-7 or IL-15); and at least one T memory stem cell (TMSC) inducer (such as TWS119).

In some embodiments, the microparticles disclosed herein comprise at least one immunogen (such as a COVID spike protein), at least one immunostimulatory cytokine (such as IL-2, IL-7 or IL-15); at least one T memory stem cell (TMSC) inducer (such as TWS119), and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein), at least one immunostimulatory cytokine (such as IL-2, IL-7 or IL-15); at least one T memory stem cell (TMSC) inducer (such as TWS119); and at least one adjuvant (such as alum, CpG, poly (I:C) or any combination thereof).

In some embodiments, the microparticles disclosed herein comprise at least one immunogen (such as a COVID spike protein), at least one immunostimulatory cytokine (such as IL-2, IL-7 or IL-15); at least one T memory stem cell (TMSC) inducer (such as TWS119), at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein); at least one T memory stem cell (TMSC) inducer (such as IL-7 or IL-15).

In some embodiments, the microparticles disclosed herein comprise at least one immunogen (such as a COVID spike protein); at least one T memory stem cell (TMSC) inducer (such as IL-7 or IL-15); and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein); at least one T memory stem cell (TMSC) inducer (such as IL-7 or IL-15); at least one T cell activator (such as anti-CD3 antibody and/or anti-CD28 antibody); and at least one adjuvant (such as alum, CpG, poly (I:C) or any combination thereof).

In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokine IL-2; and at least the T memory stem cell (TMSC) inducer, TWS119.

In some embodiments, the microparticles disclosed herein comprise at least one immunogen (such as a COVID spike protein), at least the immunostimulatory cytokine IL-2; at least the T memory stem cell (TMSC) inducer TWS119, and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokine IL-2; at least the T memory stem cell (TMSC) inducer TWS119; and at least one adjuvant (such as alum, CpG, poly (I:C) or any combination thereof).

In some embodiments, the microparticles disclosed herein comprise at least one immunogen (such as a COVID spike protein), at least the immunostimulatory cytokine IL-2; at least the T memory stem cell (TMSC) inducer TWS119, at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokine IL-7; and at least the T memory stem cell (TMSC) inducer, TWS119.

In some embodiments, the microparticles disclosed herein comprise at least one immunogen (such as a COVID spike protein), at least the immunostimulatory cytokine IL-7; at least the T memory stem cell (TMSC) inducer TWS119, and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokine IL-7; at least the T memory stem cell (TMSC) inducer TWS119; and at least one adjuvant (such as alum, CpG, poly (I:C) or any combination thereof).

In some embodiments, the microparticles disclosed herein comprise at least one immunogen (such as a COVID spike protein), at least the immunostimulatory cytokine IL-7; at least the T memory stem cell (TMSC) inducer TWS119, at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokine IL-15; and at least the T memory stem cell (TMSC) inducer, TWS119.

In some embodiments, the microparticles disclosed herein comprise at least one immunogen (such as a COVID spike protein), at least the immunostimulatory cytokine IL-15; at least the T memory stem cell (TMSC) inducer TWS119, and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokine IL-15; at least the T memory stem cell (TMSC) inducer TWS119; and at least one adjuvant (such as alum, CpG, poly (I:C) or any combination thereof).

In some embodiments, the microparticles disclosed herein comprise at least one immunogen (such as a COVID spike protein), at least the immunostimulatory cytokine IL-15; at least the T memory stem cell (TMSC) inducer TWS119, at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokines IL-2 and IL-7; and at least the T memory stem cell (TMSC) inducer, TWS119.

In some embodiments, the microparticles disclosed herein comprise at least one immunogen (such as a COVID spike protein), at least the immunostimulatory cytokines IL-2 and IL-7; at least the T memory stem cell (TMSC) inducer TWS119, and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokines IL-2 and IL-7; at least the T memory stem cell (TMSC) inducer TWS119; and at least one adjuvant (such as alum, CpG, poly (I:C) or any combination thereof).

In some embodiments, the microparticles disclosed herein comprise at least one immunogen (such as a COVID spike protein), at least the immunostimulatory cytokines IL-2 and IL-7; at least the T memory stem cell (TMSC) inducer TWS119, at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokines IL-2 and IL-15; and at least the T memory stem cell (TMSC) inducer, TWS119.

In some embodiments, the microparticles disclosed herein comprise at least one immunogen (such as a COVID spike protein), at least the immunostimulatory cytokines IL-2 and IL-15; at least the T memory stem cell (TMSC) inducer TWS119, and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokines IL-2 and IL-15; at least the T memory stem cell (TMSC) inducer TWS119; and at least one adjuvant (such as alum, CpG, poly (I:C) or any combination thereof).

In some embodiments, the microparticles disclosed herein comprise at least one immunogen (such as a COVID spike protein), at least the immunostimulatory cytokines IL-2 and IL-15; at least the T memory stem cell (TMSC) inducer TWS119, at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokines IL-7 and IL-15; and at least the T memory stem cell (TMSC) inducer, TWS119.

In some embodiments, the microparticles disclosed herein comprise at least one immunogen (such as a COVID spike protein), at least the immunostimulatory cytokines IL-7 and IL-15; at least the T memory stem cell (TMSC) inducer TWS119, and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokines IL-7 and IL-15; at least the T memory stem cell (TMSC) inducer TWS119; and at least one adjuvant (such as alum, CpG, poly (I:C) or any combination thereof).

In some embodiments, the microparticles disclosed herein comprise at least one immunogen (such as a COVID spike protein), at least the immunostimulatory cytokines IL-7 and IL-15; at least the T memory stem cell (TMSC) inducer TWS119, at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokines IL-2, IL-7 and IL-15; and at least the T memory stem cell (TMSC) inducer, TWS119.

In some embodiments, the microparticles disclosed herein comprise at least one immunogen (such as a COVID spike protein), at least the immunostimulatory cytokines IL-2, IL-7 and IL-15; at least the T memory stem cell (TMSC) inducer TWS119, and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein), at least the immunostimulatory cytokines IL-2, IL-7 and IL-15; at least the T memory stem cell (TMSC) inducer TWS119; and at least one adjuvant (such as alum, CpG, poly (I:C) or any combination thereof).

In some embodiments, the microparticles disclosed herein comprise at least one immunogen (such as a COVID spike protein), at least the immunostimulatory cytokines IL-2, IL-7 and IL-15; at least the T memory stem cell (TMSC) inducer TWS119, at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein); at least the T memory stem cell (TMSC) inducer IL-7.

In some embodiments, the microparticles disclosed herein comprise at least one immunogen (such as a COVID spike protein); at least the T memory stem cell (TMSC) inducer IL-7; and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein); at least the T memory stem cell (TMSC) inducer IL-7; at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein); at least the T memory stem cell (TMSC) inducer IL-15.

In some embodiments, the microparticles disclosed herein comprise at least one immunogen (such as a COVID spike protein); at least the T memory stem cell (TMSC) inducer IL-15; and at least one T cell activator (such as anti-CD3 antibody and/or anti-CD28 antibody).

In some embodiments the T cell activator is the combination of anti-CD3 antibody and anti-CD28 antibody.

In some embodiments, the microparticles disclosed herein comprise at least one immunogen (such as a COVID spike protein); at least the T memory stem cell (TMSC) inducer IL-15; at least one T cell activator (such as anti-CD3 antibody and/or anti-CD28 antibody); and at least one adjuvant (such as alum, CpG, poly (I:C) or any combination thereof). In some embodiments the T cell activator is the combination of anti-CD3 antibody and anti-CD28 antibody.

In some embodiments, the microparticles disclosed herein comprise at least one immunogen (such as a COVID spike protein); at least the T memory stem cell (TMSC) inducers IL-7 and IL-15.

In some embodiments, the microparticles disclosed herein comprise at least one immunogen (such as a COVID spike protein); at least the T memory stem cell (TMSC) inducers IL-7 and IL-15; and at least one T cell activator (such as anti-CD3 antibody and/or anti-CD28 antibody). In some embodiments the T cell activator is the combination of anti-CD3 antibody and anti-CD28 antibody.

In some embodiments, the microparticles disclosed herein comprise at least one immunogen (such as a COVID spike protein); at least the T memory stem cell (TMSC) inducers IL-7 and IL-15; at least one T cell activator (such as anti-CD3 antibody and/or anti-CD28 antibody); and at least one adjuvant (such as alum, CpG, poly (I:C) or any combination thereof).

In some embodiments the T cell activator is the combination of anti-CD3 antibody and anti-CD28 antibody.

In any of the foregoing embodiments, any immunogens such as but not to those described herein may be used in the microparticle compositions. In some embodiments, two or more immunogens may be used together.

Definitions. Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

Throughout this application, various disclosed embodiments may be presented in a range format. It should be understood that the description in range format is merely for convenience and brevity and should not be construed as an inflexible limitation on the scope. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present disclosure, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys. The term “higher vertebrates” is used herein and includes avians (birds) and mammals. The compositions described herein can be used to treat any suitable mammal, including primates, such as monkeys and humans, horses, cows, cats, dogs, rabbits, sheep, goats, pigs, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present disclosure and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine, or porcine. In another embodiment, the subject is mammalian.

Treatment may comprise administering a composition disclosed herein to a subject, or treatment may comprise exposing blood, blood components or T cells to a composition disclosed herein, then infusing the blood, blood components or T cells into a subject. Treatment may comprise such ex vivo methods in combination with administering a composition disclosed herein to the subject or to a site within the subject. Such ex vivo methods may comprise a patient's own blood, blood components or T cells, or may comprise blood, blood components or T cells from a donor, a cell line, or any other source.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

“Cytokines” are a category of small proteins (˜5-20 kDa) critical to cell signaling. Cytokines are peptides and usually are unable to cross the lipid bilayer of cells to enter the cytoplasm. Among other functions, cytokines may be involved in autocrine, paracrine and endocrine signaling as immunomodulating agents. Cytokines may be pro-inflammatory or anti-inflammatory. Cytokines include, but are not limited to, chemokines (cytokines with chemotactic activities), interferons, interleukins (ILs; cytokines made by one leukocyte and acting on one or more other leukocytes), lymphokines (produced by lymphocytes), monokines (produced by monocytes), and tumor necrosis factors. Cells producing cytokines include, but are not limited to, immune cells (e.g., macrophages, B lymphocytes, T lymphocytes and mast cells), as well as endothelial cells, fibroblasts, and various stromal cells. A particular cytokine may be produced by more than one cell type.

A skilled artisan would appreciate that the term “cytokine” may encompass cytokines beneficial to enhancing an immune response targeted against a cancer or a pre-cancerous or non-cancerous tumor or lesion. A skilled artisan would also appreciate that the term “cytokine” may encompass cytokines beneficial to enhancing an immune response against a disease or inflammation (e.g., resulting from surgery, an injury, or damage from an autoimmune response) or that the term “cytokine” may encompass cytokines beneficial to reducing an abnormal autoimmune response.

In some embodiments, a cytokine encoded by the nucleic acid expands and maintains T-helper cells (helper T cells). In some embodiments, a cytokine encoded by the nucleic acid expands T-helper cells. In some embodiments, a cytokine encoded by the nucleic acid maintains T-helper cells. In some embodiments, a cytokine encoded by the nucleic acid expands cytotoxic T cells (CTLs). In some embodiments, a cytokine encoded by the nucleic acid activates cytotoxic T cells. In some embodiments, a cytokine encoded by the nucleic acid expands and activates cytotoxic T cells. In some embodiments, a cytokine encoded by the nucleic acid increases proliferation of a T-helper cell population. In some embodiments, a cytokine encoded by the nucleic acid increases proliferation of a cytotoxic T cell population.

In some embodiments, the encoded cytokine comprises an interleukin (IL). A skilled artisan would appreciate that interleukins comprise a large family of molecules, including, but not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36.

In some embodiments, the encoded interleukin comprises an IL-2, IL-4, IL-6, IL-7, IL-10, IL-12, or an IL-15, or any combination thereof. In some embodiments, the encoded cytokine comprises an IL-2. In some embodiments, the encoded cytokine comprises an IL-12. In some embodiments, the encoded cytokine comprises an IL-15.

In some embodiments, the IL-2 cytokine comprises an IL-2 superkine (super IL-2 cytokine). IL-2 is a 133 amino acid glycoprotein with one intramolecular disulfide bond and variable glycosylation.

“IL-2 superkine” or “Super 2” (Fc) is an artificial variant of IL-2 containing mutations at positions L80F/R81D/L85V/I86V/I92F. These mutations are located in the molecule's core that acts to stabilize the structure and to give it a receptor-binding conformation mimicking native IL-2 bound to CD25. These mutations effectively eliminate the functional requirement of IL-2 for CD25 expression and elicit proliferation of T cells. Compared to IL-2, the IL-2 superkine induces superior expansion of cytotoxic T cells, leading to improved antitumor responses in vivo, and elicits proportionally less toxicity by lowering the expansion of T regulatory cells and reducing pulmonary edema. Examples of IL-2 superkine (Super2) deoxyribonucleic acid (DNA) and protein sequences can be found, e.g., in Table 1.

A “T cell” is characterized and distinguished by the T cell receptor (TCR) on the surface. A T cell is a type of lymphocyte that arises from a precursor cell in the bone marrow before migrating to the thymus, where it differentiates into one of several kinds of T cells. Differentiation continues after a T cell has left the thymus. A “cytotoxic T cell” (CTL) is a CD8+ T cell able to kill, e.g., virus-infected cells or cancer cells. A “T helper cell” is a CD4+ T cell that interacts directly with other immune cells (e.g., regulatory B cells) and indirectly with other cells to recognize foreign cells to be killed. “Regulatory T cells” (T regulatory cells; Treg), also known as “suppressor T cells,” enable tolerance and prevent immune cells from inappropriately mounting an immune response against “self,” but may be co-opted by cancer or other cells. In autoimmune disease, “self-reactive T cells” mount an immune response against “self” that damages healthy, normal cells.

In some embodiments, the porous scaffold further comprises a T cell immunostimulatory compound and/or a compound that induces TMSCs.

T cell immunostimulatory compounds include, but are not limited to, T cell activators, T cell attractants, or T cell adhesion compounds. T cell immunostimulatory compounds include, but are not limited to, cytokines, a therapeutic or diagnostic protein, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, a nucleic acid, chemokine ligands, and anti-CD antibodies or fragments thereof. Non-limiting examples include interleukins (e.g., IL-2, IL4, I-L6, IL-7, IL-10, IL-12, or IL-15, or an IL-2 superkine), chemokine ligands (e.g., CCL ligands, including CCL21), and anti-CD antibodies (e.g., anti-CD3 or anti-CD28) or fragments thereof, or any combination(s) thereof.

As used herein, a “targeting agent,” or “affinity reagent,” is a molecule that binds to an antigen or receptor or other molecules. In some embodiments, a “targeting agent” is a molecule that specifically binds to an antigen or receptor or other molecule. In certain embodiments, some or all of a targeting agent is composed of amino acids (including natural, non-natural, and modified amino acids), nucleic acids, or saccharides. In certain embodiments, a “targeting agent” is a small molecule.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, C-gamma-1 (Cγ1), C-gamma-2 (Cγ2), and C-gamma-3 (Cγ3). In each pair, the light and heavy chain variable regions (VL and VH) are together responsible for binding to an antigen, and the constant regions (CL, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to VH, Cγ1, Cγ2, Cγ3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

As used herein, the term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. As used herein, the term “modified immunoglobulin G” refers to a molecule that is derived from an antibody of the “G” class. As used herein, the term “antibody” refers to a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes or classes, respectively.

The term “antibody” is meant to include full-length antibodies, and may refer to a natural antibody from any organism, an engineered antibody, or an antibody generated recombinantly for experimental, therapeutic, or other purposes as further defined below. Furthermore, full-length antibodies comprise conjugates as described and exemplified herein. As used herein, the term “antibody” comprises monoclonal and polyclonal antibodies. Antibodies can be antagonists, agonists, neutralizing, inhibitory, or stimulatory. Specifically included within the definition of “antibody” are full-length antibodies described and exemplified herein. By “full length antibody” herein is meant the structure that constitutes the natural biological form of an antibody, including variable and constant regions.

The “variable region” of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

Furthermore, antibodies may exist in a variety of other forms including, for example, Fv, Fab, and (Fab′)2, as well as bi-functional (i.e., bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)).

The term “epitope” as used herein refers to a region of the antigen that binds to the antibody or antigen-binding fragment. It is the region of an antigen recognized by a first antibody wherein the binding of the first antibody to the region prevents binding of a second antibody or other bivalent molecule to the region. The region encompasses a particular core sequence or sequences selectively recognized by a class of antibodies. In general, epitopes are comprised by local surface structures that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

As used herein, the term “Fc domain” encompasses the constant region of an immunoglobulin molecule. The Fc region of an antibody interacts with a number of Fc receptors and ligands, imparting an array of important functional capabilities referred to as effector functions, as described herein. For IgG the Fc region comprises Ig domains CH2 and CH3. An important family of Fc receptors for the IgG isotype are the Fc gamma receptors (FcγRs). These receptors mediate communication between antibodies and the cellular arm of the immune system.

As used herein, the term “Fab domain” encompasses the region of an antibody that binds to antigens. The Fab region is composed of one constant and one variable domain of each of the heavy and the light chains.

In one embodiment, the term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:
(1) Fab, the fragment which contains a monovalent antigen-binding fragment of an antibody molecule, which can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, a genetically engineered fragment containing the variable region of the light chain and the variable region of the heavy chain expressed as two chains; and
(5) Single chain antibody (“SCA”), a genetically engineered molecule containing the variable region of the light chain and the variable region of the heavy chain, linked by a suitable polypeptide linker as a genetically fused single chain molecule.
(6) scFv-Fc, is produced in one embodiment, by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tri(a)body, a di- or tri-tandem scFv, a scFv-Fc bivalent molecule, an Fab, Fab′, Fv, F(ab′)2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

As used herein, the terms “bivalent molecule” or “BV” refer to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983); Morley, J. S., “Trends Pharm Sci.” (1980) pp. 463-468; Hudson et al., hit. J. Pept. Prot. Res. (1979) 14, 177-185; Spatola et al., Life Sci. (1986) 38, 1243-1249; Hann, M. M., J. Chem. Soc. Perkin Trans. I (1982) 307-314; Almquist et al., J. Med. Chem. (1980) 23, 1392-1398; Jennings-White et al., Tetrahedron Lett. (1982) 23, 2533; Szelke et al., European Application EP 45665; Chemical Abstracts 97, 39405 (1982); Holladay, et al., Tetrahedron Lett. (1983) 24, 4401-4404; and Hruby, V. J., Life Sci. (1982) 31, 189-199).

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (KD) is less than about 1×10-5 M or less than about 1×10-6 M or 1×10-7 M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In one embodiment, an antibody disclosed herein comprises a stabilized hinge region. The term “stabilized hinge region” will be understood to mean a hinge region that has been modified to reduce Fab arm exchange or the propensity to undergo Fab arm exchange or formation of a half-antibody or a propensity to form a half-antibody. “Fab arm exchange” refers to a type of protein modification for human immunoglobulin, in which a human immunoglobulin heavy chain and attached light chain (half-molecule) is swapped for a heavy-light chain pair from another human immunoglobulin molecule. Thus, human immunoglobulin molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half-antibody” forms when a human immunoglobulin antibody dissociates to form two molecules, each containing a single heavy chain and a single light chain. In one embodiment, the stabilized hinge region of human immunoglobulin comprises a substitution in the hinge region.

In one embodiment, the term “hinge region” as used herein refers to a proline-rich portion of an immunoglobulin heavy chain between the Fc and Fab regions that confers mobility on the two Fab arms of the antibody molecule. It is located between the first and second constant domains of the heavy chain. The hinge region includes cysteine residues which are involved in inter-heavy chain disulfide bonds. In one embodiment, the hinge region includes cysteine residues which are involved in inter-heavy chain disulfide bonds.

In one embodiment, the antibody or antigen-binding fragment binds its target with a KD of 0.1 nM-10 mM. In one embodiment, the antibody or antigen-binding fragment binds its target with a KD of 0.1 nM-1 mM. In one embodiment, the antibody or antigen-binding fragment binds its target with a KD within the 0.1 nM range. In one embodiment, the antibody or antigen-binding fragment binds its target with a KD of 0.1-2 nM. In another embodiment, the antibody or antigen-binding fragment binds its target with a KD of 0.1-1 nM. In another embodiment, the antibody or antigen-binding fragment binds its target with a KD of 0.05-1 nM. In another embodiment, the antibody or antigen-binding fragment binds its target with a KD of 0.1-0.5 nM. In another embodiment, the antibody or antigen-binding fragment binds its target with a KD of 0.1-0.2 nM.

In some embodiments, the antibody or antigen-binding fragment thereof provided herein comprises a modification. In another embodiment, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In some embodiments, the modification is one as further defined herein below. In some embodiments, the modification is a N-terminus modification. In some embodiments, the modification is a C-terminal modification. In some embodiments, the modification is an N-terminus biotinylation. In some embodiments, the modification is a C-terminus biotinylation. In some embodiments, the secretable form of the antibody or antigen-binding fragment comprises an N-terminal modification that allows binding to an Immunoglobulin (Ig) hinge region. In some embodiments, the Ig hinge region is from but is not limited to, an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises a C-terminal modification that allows binding to an enzymatically biotinylatable site. In some embodiments, biotinylation of said site functionalizes the site to bind to any surface coated with streptavidin, avidin, avidin-derived moieties, or a secondary reagent.

It will be appreciated that the term “modification” can encompass an amino acid modification such as an amino acid substitution, insertion, and/or deletion in a polypeptide sequence.

Non-limiting examples of antibodies, antibody fragments and antigen-binding proteins include single-chain antibodies such as scFvs.

### Methods of Making Scaffolds

In another aspect, a method is provided herein for making a porous biocompatible or biodegradable scaffold for stimulating TMSCs, the method comprising: providing a porous scaffold comprising a polymer and optionally embedding in the scaffold one or more nanoparticles. In some embodiments, the porous biocompatible or biodegradable scaffold comprising a polymer comprising alginate, hyaluronic acid, chitosan, or a combination thereof, or an arginine-glycine-aspartate (RGD) peptide, or an alginate-RGD polymer; the one or more microparticles comprising silica-heparin; or the nanoparticles comprising poly(lactic-co-glycolic acid) (PLGA). In some embodiments, the porous biocompatible or biodegradable scaffold further comprising anti-CD3 or anti-CD28 antibodies covalently bound to the polymer. In some embodiments, the at least one compound that regulates T cell immune response comprising a T cell immunostimulatory compound comprising a cytokine, a therapeutic or diagnostic protein, a growth factor, a chemokine, a therapeutic or diagnostic antibody or fragment thereof, an antigen-binding protein, a Fc fusion protein, interleukin-2 (IL-2), interleukin-4 (IL-4), interleukin-6 (IL-6), interleukin-7 (IL-7), interleukin-10 (IL-10), interleukin-12 (IL-12), interleukin-15 (IL-15), IL-2 superkine, chemokine (C-C motif) ligand 21 (CCL21), anti-CD3 or anti-CD28, or any combination thereof; or the at least one compound that regulates induction of TMSCs comprising a compound such as TWS119.

In some embodiments, the alginate matrix, once combined with each component, is cross-linked with calcium and microparticles prepared therefrom.

Implantable scaffolds are made of various biocompatible and biodegradable polymers, such as alginate, hyaluronic acid, and chitosan. Microscale pores may be created within the structures. To create scaffolds with stimulatory capability by this artificial niche, mesoporous silica microparticles may be embedded in the scaffolds.

The implantable scaffold can be made of various biocompatible and biodegradable polymers.

### Methods of Using Scaffolds

The microparticle scaffolds described herein can be fabricated for various applications for therapeutic or preventative vaccination or inducing an immune response, in one embodiment, in subjects who are immunosenescent, immunocompromised, or undergoing cancer therapy. They can be used to enhance the production of CAR-T and other engineered cells for immunotherapy and other purposes. They can be used to provide an improved cancer vaccine. In one aspect, the porous scaffold is provided at a site at or near a focus of interest in a subject in need. In one embodiment the scaffold biodegrades. In some embodiment the mechanical properties of the scaffold as well as the degradation time can be modified for a particular use by changing the formulation.

In some embodiments, “treating” comprises therapeutic treatment including prophylactic or preventive measures, wherein the object is to prevent or lessen the targeted pathologic condition or disorder, for example to treat or prevent cancer. Thus, in some embodiments, treating may include directly affecting or curing, suppressing, inhibiting, preventing, reducing the severity of, delaying the onset of, reducing symptoms associated with cancer or a combination thereof. Thus, in other embodiments, treating may include directly affecting or curing, suppressing, inhibiting, preventing, reducing the severity of, delaying the onset of, reducing symptoms associated with a non-cancerous tumor or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A “cancer” is one of a group of diseases characterized by uncontrollable growth and having the ability to invade normal tissues and to metastasize to other parts of the body. Cancers have many causes, including, but not limited to, diet, alcohol consumption, tobacco use, environmental toxins, heredity, and viral infections. In most instances, multiple genetic changes are required for the development of a cancer cell. Progression from normal to cancerous cells involves a number of steps to produce typical characteristics of cancer including, e.g., cell growth and division in the absence of normal signals and/or continuous growth and division due to failure to respond to inhibitors thereof; loss of programmed cell death (apoptosis); unlimited numbers of cell divisions (in contrast to a finite number of divisions in normal cells); aberrant promotion of angiogenesis; and invasion of tissue and metastasis.

A “pre-cancerous” condition, lesion, or tumor is a condition, lesion, or tumor comprising abnormal cells associated with a risk of developing cancer. Non-limiting examples of pre-cancerous lesions include colon polyps (which can progress into colon cancer), cervical dysplasia (which can progress into cervical cancer), and monoclonal monopathy (which can progress into multiple myeloma). Premalignant lesions comprise morphologically atypical tissue which appears abnormal when viewed under the microscope, and which are more likely to progress to cancer than normal tissue.

A “non-cancerous tumor” or “benign tumor” is one in which the cells demonstrate normal growth, but are produced, e.g., more rapidly, giving rise to an “aberrant lump” or “compact mass,” which is typically self-contained and does not invade tissues or metastasize to other parts of the body. Nevertheless, a non-cancerous tumor can have devastating effects based upon its location (e.g., a non-cancerous abdominal tumor that prevents pregnancy or causes a ureter, urethral, or bowel blockage, or a benign brain tumor that is inaccessible to normal surgery and yet damages the brain due to unrelieved pressure as it grows).

In some embodiments, “treating” comprises therapeutic treatment including prophylactic or preventive measures, wherein the object is to prevent or lessen the targeted pathologic condition or disorder, for example to treat or prevent an autoimmune disease, an allergic reaction or hypersensitivity reaction, a localized infection or an infectious disease, an injury or other damage, a transplant or other surgical site, or a symptom thereof, or a combination thereof. Thus, in some embodiments, treating may include directly affecting or curing, suppressing, inhibiting, preventing, reducing the severity of, delaying the onset of, reducing symptoms associated with an autoimmune disease, an allergic reaction or hypersensitivity reaction, a localized infection or an infectious disease, an injury or other damage, a transplant or other surgical site, or a symptom thereof, or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In some embodiments, application of the scaffold, or compositions thereof is for local use. This may, in certain embodiments, provide an advantage, wherein the controlled localized release of the compound regulating T cell immune response may provide a local immune effect thereby avoiding a toxic systemic effect of the cytokine. In one example, controlled release of IL-2 or similar cytokine, may increase proliferation of cytotoxic T cells and or helper T cells in the area adjacent to the cancer or tumor, thereby promoting clearance of the cancer or tumor. In some embodiments, controlled release of IL-2 or similar cytokine, may maintain a helper T cell population in the area adjacent to the tumor. In some embodiments, controlled release of IL-2 or similar cytokine, may activate a cytotoxic T cell population in the area adjacent to the tumor. In some embodiments, controlled release of IL-2 or similar cytokine, may lead to enhanced killing of tumor cells in the localized area at and adjacent to the tumor. In some embodiments, controlled release of IL-2 or similar cytokine, provides enhanced clearance of a tumor. This technique may also be used for the treatment of other diseases, reactions, injuries, transplants, blood clots, and the like, recited herein.

As used herein, the terms “composition” and “pharmaceutical composition” may in some embodiments, be used interchangeably having all the same qualities and meanings. In some embodiments, disclosed herein is a pharmaceutical composition for the treatment of a cancer or tumor as described herein. In some embodiments, disclosed herein is a pharmaceutical composition for the treatment of cancer or tumor. In some embodiments, disclosed herein is a pharmaceutical composition for the use in methods locally regulating an immune response. In some embodiments, disclosed herein are pharmaceutical compositions for the treatment of an autoimmune disease, an allergic reaction or hypersensitivity reaction, a localized site of an infection or infectious disease, a localized site of an injury or other damage, a transplant or other surgical site, a blood clot causing or at risk for causing a myocardial infarction, an ischemic stroke, or a pulmonary embolism or a symptom thereof, or a combination thereof.

In some embodiments, a pharmaceutical composition comprises a microparticle as described in detail above. The microparticle composition may be formulation for ease in administration, such as subcutaneous injection or deposition. The microparticles may be formulated in an aqueous medium such as buffers, stabilizers, preservatives, or other components.

In some embodiments, said immunization enhanced the immune response against the antigen, whether a therapeutic (e.g., cancer) or preventative (e.g., viral) antigen. In inducing an immune response against a tumor antigen, treatment reduces the size of the tumor, eliminates the tumor, slows the growth or regrowth of the tumor, or prolongs survival of said subject or any combination thereof. For a therapeutic vaccine against an infectious agent, immunization reduces or eliminates infection or symptoms at said focus of infection or symptoms of said localized infection or infectious disease, prolongs survival of said subject, or any combination thereof; reduces, eliminates, inhibits or prevents structural, organ, tissue, or cell damage, inflammation, infection, or another symptom at said site of injury or said site of chronic damage, improves structural, organ, tissue, or cell function at said site of injury or said site of chronic damage, improves mobility of said subject, prolongs survival of said subject, or any combination thereof; reduces, eliminates, inhibits, or prevents structural, organ, tissue or cell damage, inflammation, infection, or another symptom.

In some embodiments, a pharmaceutical composition comprises the porous scaffold, as described in detail above. In still another embodiment, a pharmaceutical composition for the treatment of cancer or tumor, as described herein, comprises an effective amount of the immunogen.

In some embodiments, the localized site of an infection or the localized site of an infectious disease includes, for example, but is not limited to, a fungal infection (e.g., aspergillus, coccidioidomycosis, tinea pedis (foot), tinea corporis (body), tinea cruris (groin), tinea capitis (scalp), and tinea unguium (nail)), a bacterial infection (e.g., methicillin-resistant Staphylococcus aureus [MRSA], localized skin infections, abscesses, necrotizing facsciitis, pulmonary bacterial infections [e.g., pneumonia], bacterial meningitis, bacterial sinus infections, bacterial cellulitis, such as due to Staphylococcus aureus (MRSA), bacterial vaginosis, gonorrhea, chlamydia, syphilis, Clostridium difficile (C. diff), tuberculosis, cholera, botulism, tetanus, anthrax, pneumococcal pneumonia, bacterial meningitis, Lyme disease), a viral infection (e.g., varicella-zoster/herpes zoster [shingles], Herpes simplex I [e.g., cold sores/fever blisters], Herpes simplex II [genital herpes], or human papilloma virus [e.g., cervical cancer, throat cancer, esophageal cancer, mouse cancer], Epstein-Barr virus [e.g., nasopharyngeal cancer], encephalitis viruses [e.g., brain inflammation], or hepatitis viruses [e.g., liver disease; hepatitis A, hepatitis B, hepatitis C, hepatitis D, hepatitis E, hepatitis F, hepatitis G] or COVID-19), a parasitic infection (e.g., an area infected by scabies, Chagas, Hypoderma tarandi, amoebae, roundworm, Toxoplasma gondii). In some embodiments, the injury or other damage includes, for example, but is not limited to traumatic injury (e.g., resulting from an accident or violence) or chronic injury (e.g., osteoarthritis). In some embodiments, the localized site of injury comprises a muscular-skeletal injury, a neurological injury, an eye or ear injury, an internal or external wound, or a localized abscess, an area of mucosa that is affected (e.g., conjunctiva, sinuses, esophagus), or an area of skin that is affected (e.g., infection, autoimmunity). In some embodiments, the transplant or other surgical site includes, for example, but is not limited to, the site and/or its local environment or surroundings of an organ, corneal, skin, limb, face, or other transplant, or a surgical site and/or its local environment or surroundings, for, e.g., but not limited to, treatment of surgical trauma, treatment of a condition related to the transplant or surgery, or prevention of infection. In some embodiments, the site is at or adjacent to a blood clot causing or at risk for causing a myocardial infarction, an ischemic stroke, or a pulmonary embolism. In some embodiments, the methods disclosed herein treat one or more symptoms of a disease, reaction, infection, injury, transplant, surgery, or blood clot. In some embodiments, the methods disclosed herein treat a combination thereof.

Treatment of the subject with the microparticles may also be used in conjunction with other known treatments.

Any patent, patent application publication, or scientific publication, cited herein, is incorporated by reference herein in its entirety.

The following examples are presented in order to more fully illustrate some embodiments of the disclosure. They should, in no way be construed, however, as limiting the broad scope of the disclosure. One skilled in the art can readily devise many variations and modifications of the principles disclosed herein without departing from the scope of the disclosure.

### EXAMPLES

### Example 1. Pseudoviral Infection Study (Pseudovirus Challenge) In Vitro

In order to test the effectiveness of the proposed approach in providing immunization, SARS-CoV-2 pseudotyped lentivirus (pSARS-CoV-2; Luc-eGFP; BPS Bioscience, San Diego, CA) was used in a neutralization assay using ACE2 expressing cells in vitro, as previously described (Wong S K, Li W, Moore M J, et al. A 193-amino acid fragment of the SARS coronavirus S protein efficiently binds angiotensin-converting enzyme 2. J Biol Chem 2004; 279: 3197-3201; Nie J, Wu X, Ma J, et al. Development of in vitro and in vivo rabies virus neutralization assays based on a high-titer pseudovirus system. Sci Rep 2017; 7: 42769; He Y, Li J, Li W, et al. Cross-neutralization of human and palm civet severe acute respiratory syndrome coronaviruses by antibodies targeting the receptor-binding domain of spike protein. J Immunol 2006; 176: 6085-6092). The selected pseudotyped Lentivirus was produced by replacing the VSV-G fusion glycoprotein with SARS-CoV-2 Spike protein (Genbank #QHD43416.1) as a surrogate viral envelope protein. These pseudovirions also contain a firefly luciferase and eGFP cassette (Luc-P2A-eGFP) driven by a CMV promoter. The luciferase and eGFP are co-expressed under the CMV promoter in the transduced cells. Therefore, the Spike-mediated entry into the target cell can be conveniently measured via luciferase reporter activity or eGFP expression in a Biosafety Level 2 facility. To test neutralizing antibody responses, serum from mice (immunized and control) were preincubated with pseudotyped lentivirus at various dilutions. After incubation for 1 h at 37° C., the mixture will be added to ACE2/HEK293 cell (#79951; BPS Bioscience, San Diego, CA) to detect viral infectivity. Media will be changed the following day and 48 h after infection, eGFP signal in infected cells will be determined by fluorescent microscopy and flow cytometry.

The immunomodulatory scaffold is able to boost TMSCs in animals: there may be difference in TMSC induction and antibody formation in young and old mice.

### Example 2. Injectable Scaffold to Promote the Formation of SARS-CoV-2 Antigen-Specific TMSCs In Vitro

Augmentation and engineering of the immune response requires more than just the delivery of antigens. The control of activation, differentiation and maturation of antigen-specific T cells is a key step in the generation of long-term immunity. In this approach, injectable immunomodulatory scaffold is shown to recruit T cells and induce antigen specific TMSCs. The schematic design of the scaffold is shown in FIG. 35. Immunomodulatory scaffolds were developed that provide the essential ingredients for efficient TMSC induction in vitro and in vivo. 3D microenvironment can mimic the in vivo context encountered by activated lymphocytes.

Scaffolds based on heparinized alginate feature enhanced affinity toward selected cytokines (Majedi F S, Hasani-Sadrabadi M M, Kidani Y, et al. Cytokine Secreting Microparticles Engineer the Fate and the Effector Functions of T-Cells. Advanced Materials 2018; 30: 1703178), and was used as the scaffold material. Chemokines can be released from this scaffold to recruit T cells. Alginate scaffolds with various mechanical stiffnesses were prepared while presenting polyclonal or antigen-specific signals to activate primary T cells (Majedi F S, Hasani-Sadrabadi M M, Thauland T J, et al. T-cell activation is modulated by the 3D mechanical microenvironment. Biomaterials 2020; 252: 120058.). Desired targeting molecules (e.g., CD3 and CD28 antibodies) conjugated to the scaffold, together with soluble cytokine release (e.g., IL-2 loaded by heparin binding), to activate T cells. In addition, small molecule TWS119 (3-[[6-(3-Aminophenyl)-7H-pyrrolo[2,3-d]pyrimidin-4-yl]oxyphenol ditrifluoroacetate), a potent inhibitor of the serine-threonine glycogen synthase kinase 3β (GSK-3β), is able to induce the Wnt-β-catenin signaling and the enrichment of TMSC through differentiation inhibition both in mice and in human (Gattinoni L, Zhong X-S, Palmer D C, et al. Wnt signaling arrests effector T cell differentiation and generates CD8+ memory stem cells. Nature medicine 2009; 15: 808). TWS119 was delivered for TMSC induction. TWS119 is hydrophobic and can be loaded into poly(lactic-co-glycolic acid) (PLGA) nanoparticles for a sustained release.

The scaffold disclosed herein can also release SARS-CoV-2 antigens. Released viral antigens components will be uptake by recruited tissue immune cells and cross-presented to T cells while receiving activation signals inside the scaffolds. The presence of these antigens in addition to soluble (IL-2 and TWS119) and conjugated (anti-CD3/28) cues in the scaffold will drive tissue resident naïve T cells toward the formation of antigen-specific TMSCs. Matured immune cells will then migrated toward the draining lymph nodes (dLNs). In dLNs, adaptive immunity will form via antigen-specific expansion of T and B cells. This will cause induction in humoral as well as cellular immunity. Thus, the 3D scaffolds disclosed herein comprise the following tunable properties: (1) mechanical property, (2) release of chemokine to recruit tissue resident T cells, (3) conjugated CD3/CD28 antibodies and released IL-2 to activate naïve T cells in 3D scaffold, (4) sustained delivery of small molecule TW119 from PLGA nanoparticles loaded in alginate gel to induce naïve T cells into TMSCs, and (5) delivery of a vaccine candidate, i.e., a peptide pool created based on the Spike protein of SARS-CoV-2.

### Example 3. Methods for Scaffold Development and Evaluation

Primary T cells will be isolated from young (6-10 weeks-old) and old (10-12 month-old), and male and female wild type C57/B6 mice (Jackson Labs) for in vitro experiments. Cell-culture media will be RPMI supplemented with 10% heat-inactivated FBS, 1% penicillin/streptomycin, 1% sodium pyruvate, 1% HEPES buffer, 0.1% μM 2-mercaptoethanol. CD4+/CD8+ T cells were purified from mice spleens and lymph nodes using negative selection enrichment kits (Stem Cell Technologies) as reported before (Meng K P, Majedi F S, Thauland T J, et al. Tissue mechanics controls T-cell activation and metabolism. bioRxiv 2019: 581322.). Peripheral blood mononuclear cells (PBMCs) from healthy human donors (IRB #20-001194) were used as cell source for naïve human T cell source in this study to generate human T memory stem cells in vitro. PBMCs will be isolated by Ficoll-Hypaque gradient separation (Lymphoprep, Fresenius) and enriched for CD3+ cells with the human CD3 MicroBeads Isolation Kit (Miltenyi Biotec) following the manufacturer's instruction.

To fabricate tunable alginate 3D scaffolds, a microporous alginate-RGD scaffolds was used as a substrate for T-cell trafficking. Alginate has a good biocompatibility and controllable biodegradation, and has been widely used as base materials for tissue engineering and regenerative medicine. In some embodiments, the degree of oxidation, molecular weight, and calcium content can determine how fast the alginate-based scaffold will degrade in vitro and in vivo. The in vitro (in PBS at 37° C. on a shaker incubator) results show that the half-life of one exemplary scaffold is about 5 weeks. Scanning electron microscope (SEM) images of alginate-RGD scaffolds demonstrated their highly porous microstructure with an average pore size of 65±4 μm. These results confirm the ability to synthesize scaffolds of tunable microstructure and mechanics, which can be decorated with a wide range of different surface proteins or targeting antibodies, depending on their intended applications. In this disclosure, scaffolds were fabricated with various level of stimulatory antibodies (e.g., anti-CD3 and anti-CD28) conjugated alginate-based scaffolds that emulate lymph nodes in terms of morphology and mechanical stiffness. In addition, chemokine/cytokine can be easily loaded into alginate scaffolds conjugated with heparin through heparin binding.

As subcutaneous insertion of the scaffold in one non-limiting embodiment is difficult and not applicable in large scale vaccination, injectable hydrogels will herein be developed using a cryogelation technique as reported elsewhere. Alginate-methacrylate-RGD was developed and crosslinked chemically (e.g., following the guidance of Bencherif S A, Sands R W, Ali O A, et al. Injectable cryogel-based whole-cell cancer vaccines. Nature communications 2015; 6: 1-13; or Thanarajasingam U, Sanz L, Diaz R, et al. Delivery of CCL21 to metastatic disease improves the efficacy of adoptive T-cell therapy. Cancer research 2007; 67: 300-308, by way of non-limiting examples). Such a structure can withstand large deformations and can be easily compressed to a fraction of their sizes and passed through a surgical needle and recover its original shape afterwards as shown herein.

Physical measurement of scaffold mechanics are performed by compression testing (e.g., Instron). The physical properties of the scaffolds are altered by manipulating the alginate and calcium concentrations. In one embodiment, alginate in the range of 0.5 to 2.5 wt % and crosslinking conditions including Ca²⁺ concentration (10-40 mM) during formation allows for sampling a stiffness range of 2-60 kPa. The physiological context for using this range of stiffnesses comes from empirical measurements of lymph nodes from mice prior to infection (˜4 kPa) to a peak stiffness measured after systemic viral infection (˜40 kPa) (Meng K P, Majedi F S, Thauland T J, et al. Tissue mechanics controls T-cell activation and metabolism. bioRxiv 2019: 581322). This range of stiffnesses, in some embodiments, provides a useful scope for the studies described here, as a reasonable physiological range that T cells would be exposed to during quiescence and during an aggressive, systemic infection. To assess the physical degradation rates based on various mechanical tuning of the scaffolds, the durability of the matrices was tested in vivo after implantation of the scaffolds subcutaneously in wild-type (C57/B6) mice. The biocompatibility of scaffolds (without releasing cytokines) was also determined based on the presence of inflammatory markers including lymphocytes (CD3) and macrophages (CD68) at different time points (3, 7, 14 and 28 days) with the same subcutaneous test. The impact of altering antibodies and the mechanical stiffness was tested on the proliferation, activation markers, and cytokine production by co-culture with naïve mouse and human T cells while providing soluble cytokines required for the formation of TMSCs

### Example 4. Evaluating the Role of Chemoattractant Release on T Cell Recruitment

T cells are attracted to tissue microenvironments based on chemokine and integrin signaling among other cues. T cell recruitment into the scaffold was evaluated by loading the chemokines CCL21, CCL19, CCL17 and/or CCL22 into the heparin-conjugated alginate-RGD matrix. These chemokines are well-established to play important roles in recruiting effector T cells into sites of infection and cancer. A range of concentrations will be tested through in vitro chemotaxis studies by using two assays: (1) Transwell analysis with the scaffold (with or without the chemokine(s) in the bottom well and T cells in the insert on the top, followed by immunostaining for T cells (CD4 and CD8) at the bottom of the insert and microscopic counting; (2) scaffold (with or without chemokine(s) encapsulated in collagen gel (tissue mimetic (three-dimensional fibrillar collagen gel) with dispersed T cells (as a surrogate for natural tissue), followed by time-laps imaging to study the migration of T cells into the alginate scaffolds. After 7, days, collagen and scaffold compartments will be digested and population of T cells will be analyzed using flow cytometry.

### Example 5. Engineering the Scaffold with Signals for T Cell Activation

Anti-CD3 and CD28 antibodies were conjugated to alginate-RGD gel as immobilized T cell activation signals. In addition, heparin was conjugated to alginate to load IL-2 through heparin binding; the released IL-2 served as soluble signal for T cell activation. Heparin-functionalized alginate scaffolds were synthesized and optimized to encapsulate target cytokines IL-2. Alginate-Heparin conjugates were developed at several conjugation densities and were used instead of unmodified alginate. Carbodiimide chemistry (NHS/EDC) was utilized to modify amine-linked alginate. Heparin presence provides enhanced efficiency and stability of cytokine that enables precise spatiotemporal control over the release profile of target cytokines. Release of IL-2 was studied under gentle shaking (50 rpm) at 37° C. using ELISA kits (R&D Systems).

Naïve mouse or human T cells were cultured in 3D alginate-RGD scaffolds of various stiffness with/without CD3/CD28/IL-2. After 48 h of stimulation, the activation markers were tested by flow cytometry analysis, and cells will be cultured for 5, 7, and 14 additional days in order to evaluate the ex vivo expansion and assess the stemness of treated cells. The change in T cell activation, differentiation, and formation of TMSCs will be tested at using flow cytometry by checking the surface markers including CD4, CD8, CD62L⁺, CD45RO⁻, CD45RA⁺, CD27⁺, CD95⁺, CD122⁺, and CCR7⁺ for human T cells 8, 12 and CD4, CD8, CD62L⁺, CD44⁻, CD122⁺, and Sca-1⁺ for murine T cells while expressing intracellular IFN-γ cytokine and not IL-4. For intracellular cytokine staining (ICCS), cells were fixed with cold 4% paraformaldehyde (PFA) for 20 min at room temperature and then permeabilized with 0.5% saponin for 10 min before blocking with PBS+1% BSA+5% donkey serum and staining with appropriate antibodies at 1:100 dilution. Cells were washed and analyzed by flow cytometry as above. The following antibodies were used for ICCS from Biolegend: IL-2, IL-4, IL-10, IL-17A, IFN-γ, and TNF-α. The cells were assessed for T cells skewing using intracellular cytokine staining (ICCS) for IFN-γ and IL-4. Th1 cells should show more IFN-γ production than IL-4 (which is the major cytokine of Th2 cells). The optimal levels of stiffness/CD3/CD28/IL-2 combination for T cell activation were used for further investigations.

### Example 6. Enhance TMSC Formation Via Release of Small Molecule TWS119

The 4,6-disubstituted pyrrolopyrimidine, TWS119, was included to enhance TMSC formation. This hydrophobic small molecule inhibitor was incorporated via PLGA nanoparticles. The kinetics of TWS119 release from 3D scaffolds with various initial loading (1, 5, 10, and 20 μM) were studied in vitro using HPLC. Our observations show the ability of controlling the sustained release of TWS119 from monodisperse (PDI: 0.02) 150 nm PLGA nanoparticles. Target delivery regimes were reached for each dose by modifying the formulation and composition.

In some embodiments, the individual components of the disclosed microparticle based vaccination platform have been approved for clinical trials or therapies. In one embodiment, alginate, a biopolymer used as a base material to form microparticles, is approved by FDA for various applications. In some embodiments, GMP-grade alginate is used. In some embodiments, recombinant human CD3/CD28 antibodies are used in the microparticles disclosed herein. Such antibodies are approved for use in CAR-T cell therapy.

### Example 7. Results

FIG. 1 depicts the mechanism of action of artificial antigen presenting cells (aAPC) disclosed herein. (A) After subcutaneous injection of artificial antigen presenting cells (aAPCs) (engineered biomaterial vaccine), (B) viral-related components (Spike peptides) will be released from and uptake by tissue-resident dendric cells and macrophages. Presence of these antigens in addition to sustained release of cytokines and inhibitor as well as conjugated (anti-CD3/28) cues via aAPCs will drive tissue resident naïve T cells toward formation of antigen specific T memory stem cells (TMSCs). (C) Matured immune cells will then migrated toward the draining lymph nodes (dLNs). In dLNs, adaptive immunity will form via antigen specific expansion of T and B cells. This will cause induction in humoral as well as cellular immunity.

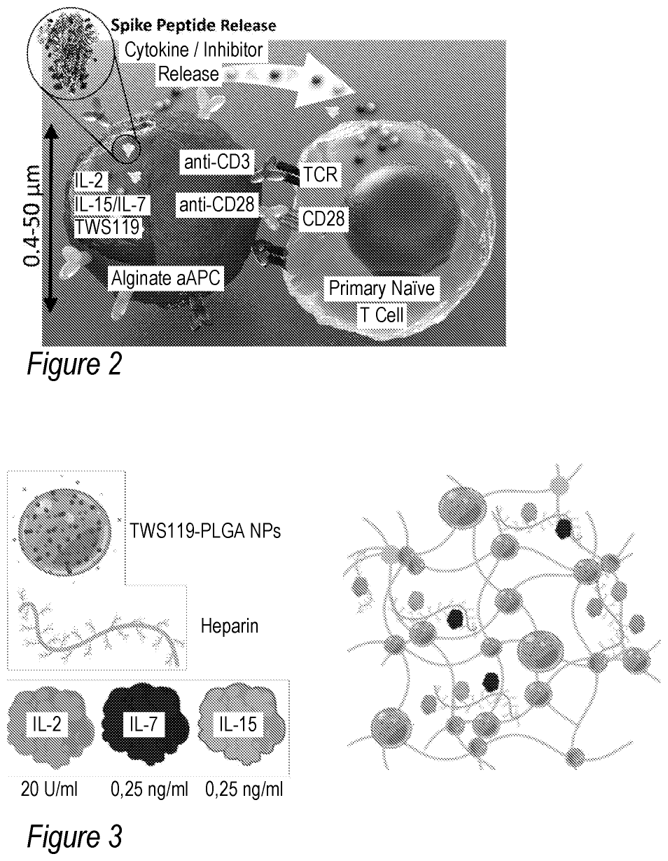

FIG. 2 is a schematic representation of artificial antigen presenting cells (aAPCs). These monodisperse particles were made using microfluidic platforms or membrane emulsification technique in size range of 0.4-50 um. Wide range of natural and synthetic polymers can be used as core materials.

FIG. 3 is an example of aAPCs formulation. Alginate conjugated with heparin will be used as core materials. Alginate is a biocompatible polysaccharide polymer which can be crosslinked via use of divalent cations like Calcium and Barium or via chemical crosslinking. Presence of heparin will increase the affinity of cytokines to the particles in natural way as it represents glycosaminoglycans (GAG). This Alginate-Heparin conjugate was tested toward encapsulation and sustained release of target cytokines (IL-2, IL-7, and IL-15). Due to hydrophobic nature of TWS119, PLGA (50:50 DL-lactide/Glycolide, PURASORB PDLG 5004A) was utilized to encapsulate this small molecule inhibitor. PLGA nanoparticles (NPs) in size range of 100-300 nm were formed and the mixed with Alginate-Heparin prior to forming microparticle hydrogels. The microparticles will be surface functionalized with anti-CD3 and anti-CD28 to provide active engagement with T cells. Adjuvants (like CpG, Poly(I:C)) will be added to the formulation (encapsulated in microparticles) for in vivo experiments. Addition of adjuvant is necessary to increase immunogenicity at the injection site.

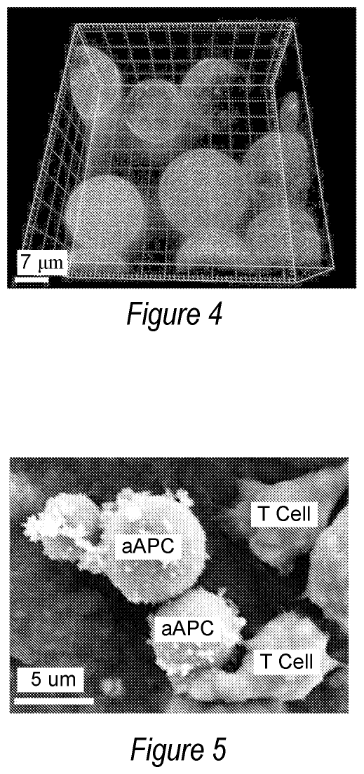

FIG. 4 depicts confocal micrograph of microparticles coated with fluorescent anti-CD3 antibody showing even distribution of antibodies on the microparticles' surface.

FIG. 5 depicts a scanning electron microscopy (SEM) picture demonstrating the interactions between primary T cells and aAPCs.

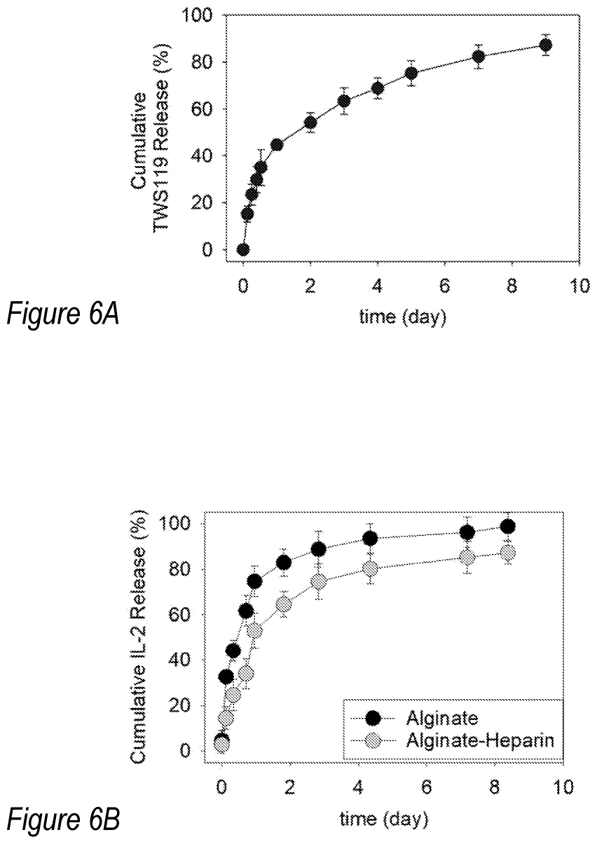

FIG. 6 shows (A) cumulative release of small molecule inhibitor (TWS119) from PLGA loaded encapsulated in aAPCs. (B) Cumulative release of interleukin cytokine (here IL-2) from Alginate and Alginate-Heparin based aAPCs as assessed by ELISA kit.

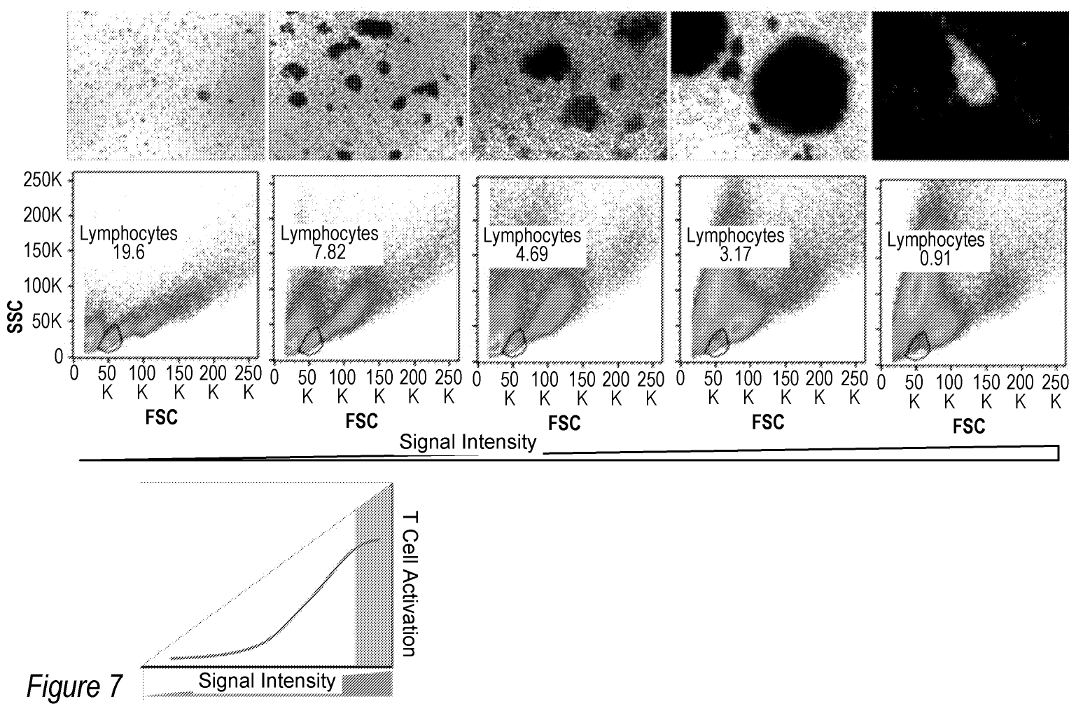

FIG. 7 shows (Top) Representative brightfield microscopy images of primary mouse T cells cultured with aAPCs modified with various density of anti-CD3 and anti-CD28. Here 5 um aAPCs were used which are based on Alginate-Heparin and loaded with IL-2. No TWS119 is presented here.

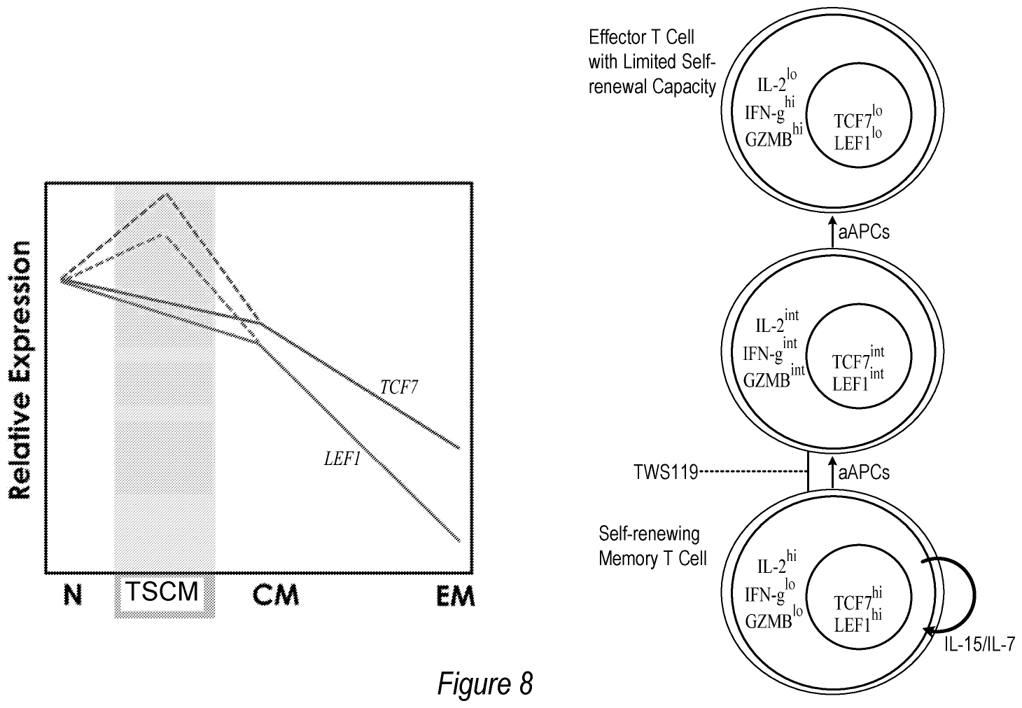

FIG. 8 shows that T cell factor (TCF) 1 and lymphoid enhancer-binding factor (LEF) 1 are downstream transcription factors of the Wnt/β-catenin signaling pathway. Based on the literatures indicate that Wnt signaling might regulate the maturation of T cells. Expression of LEF1 and TCF7, decreases with progressive differentiation of CD8+ T cells from naive (N)→central memory (CM)→effector memory (EM) in humans and mice. TWS119 can activate Wnt signaling in primary CD8+ T cells during the polyclonal activation and induce formation of T memory stem cells (TSCMs). Co-presentation of IL-7 and IL-15 can help further expansion of TSCMs in culture.

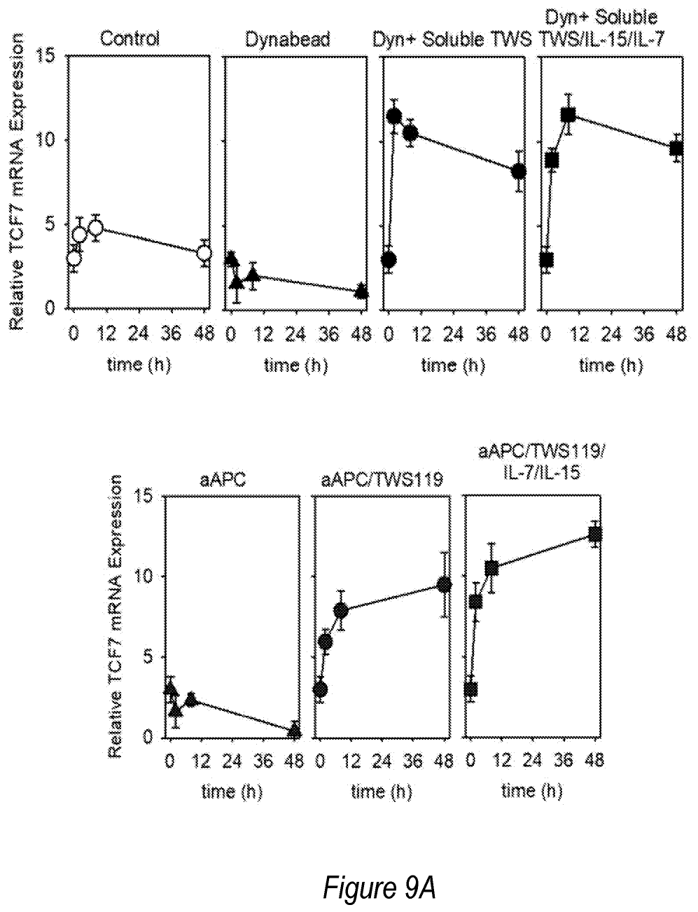

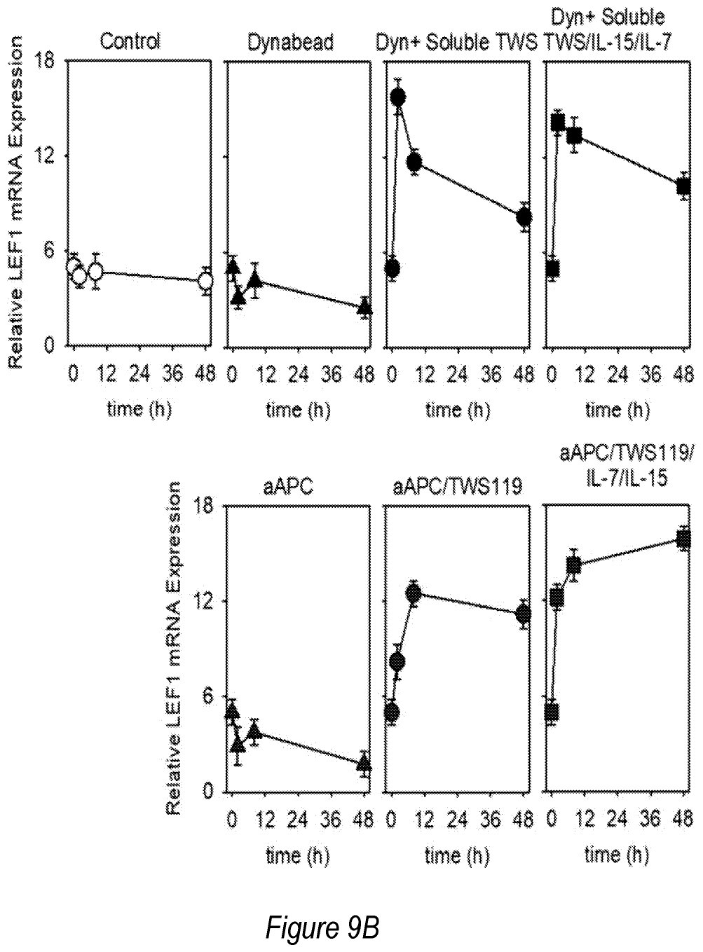

FIG. 9 shows that aAPCs delivering TWS119 can activate Wnt signaling in primary CD8+ T cells. Naive CD8+ T cells were isolated from wild-type B6 mice and then incubated with indicated formulations. Quantitative RT-PCR analysis of the expression of TCF7 and LEF1 in CD8+ T cells treated with various formulations. Data are represented as mean±SD. All data are representative of at 3-5 independently performed experiments.

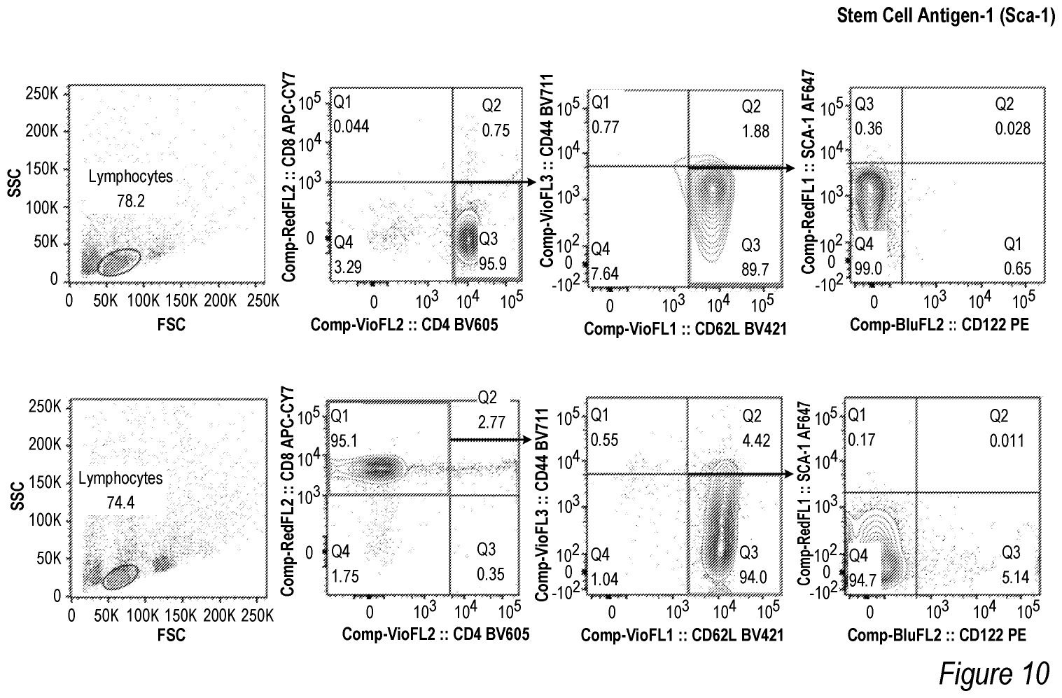

FIG. 10 depicts gating strategy showing population of TSCMs in naïve CD4+ (Top panels) and CD8+ (Lower panels) T cells. TSCMs are identified as CD62L+CD44−Sca-1+CD4/CD8 T cells. Less than 1% of naïve T cells are TSCMs.

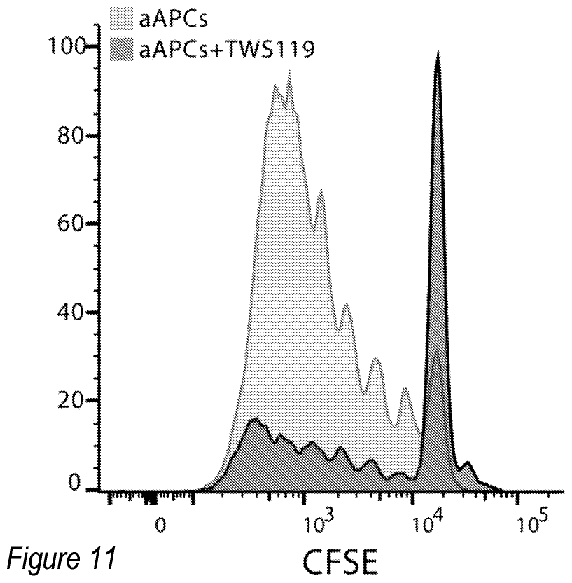

FIG. 11 shows treatment of naïve CD8+ T cells with TWS119 will limit the proliferation as studied by CFSE proliferation staining.

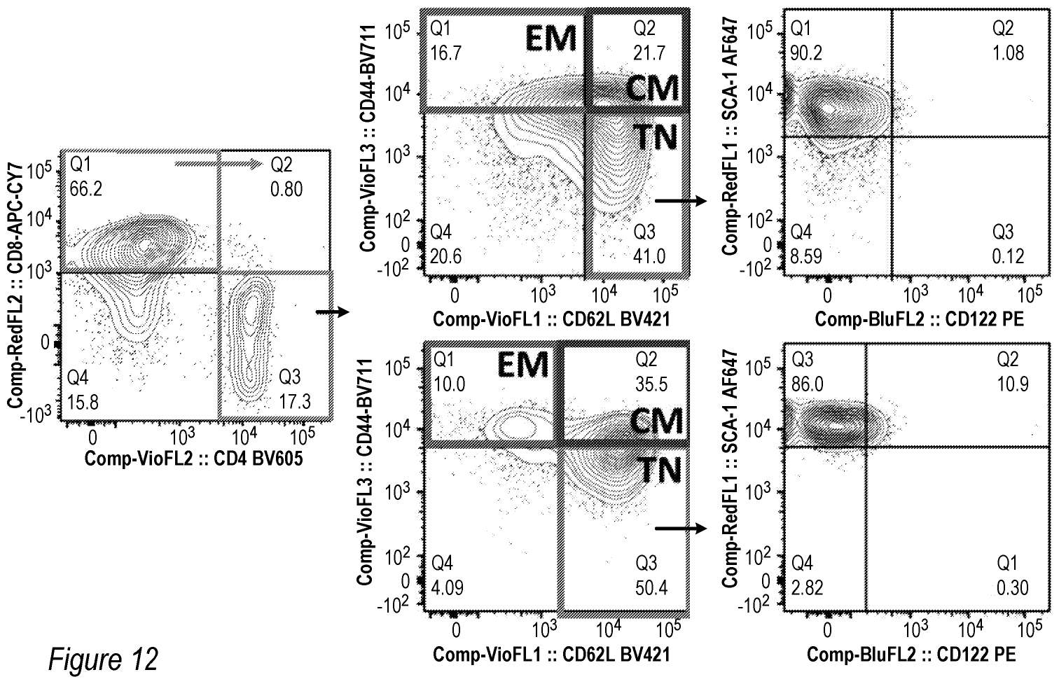

FIG. 12 depicts gating strategy showing population of TSCMs in naïve T cells treated with TWS119 containing aAPCs. Naïve (TN), central memory (CM), and effector memory (EM) T cell population were shown. CD4+(Top panels) and CD8+(Lower panels) T cells. TSCMs are identified as CD62L+CD44−Sca-1+CD4/CD8 T cells. More than 85% of naïve T cells can be differentiated into TSCMs.

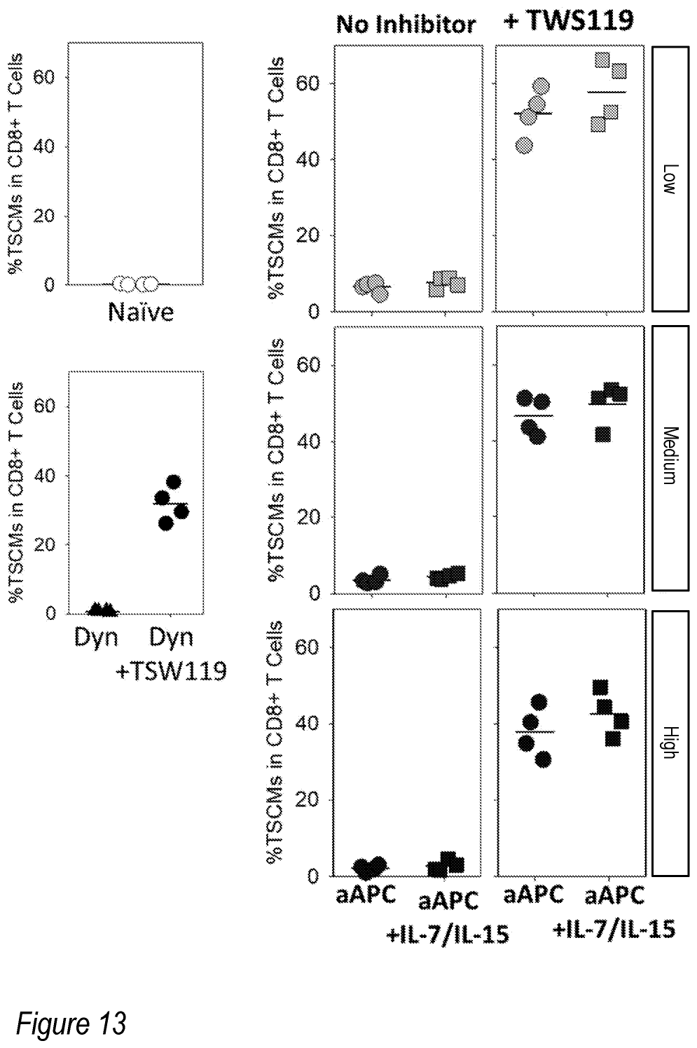

FIG. 13 shows Quantification of TMSCs in CD8+ T cells based on surface markers after four days of co-culturing with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of anti-CD3/anti-CD28 antibodies conjugated on surface of microparticles.

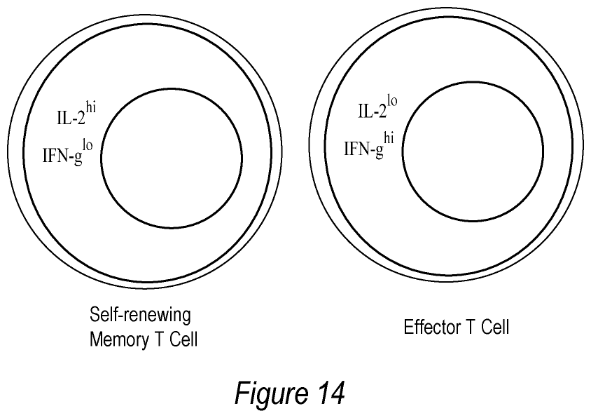

FIG. 14 shows Intracellular expression of IL-2 and IFN-g can be used as cellular markers to identify induced TSCMs.

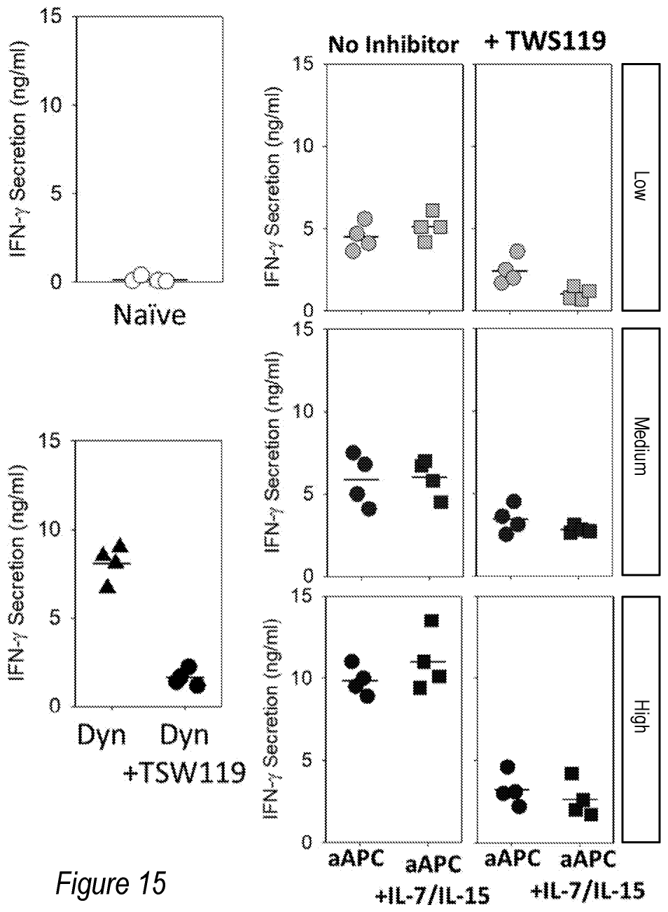

FIG. 15 shows Intracellular expression of IFN-g was used as cellular markers to quantify induced TSCMs. Naïve T cells were co-cultured with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of anti-CD3/anti-CD28 antibodies conjugated on surface of microparticles.

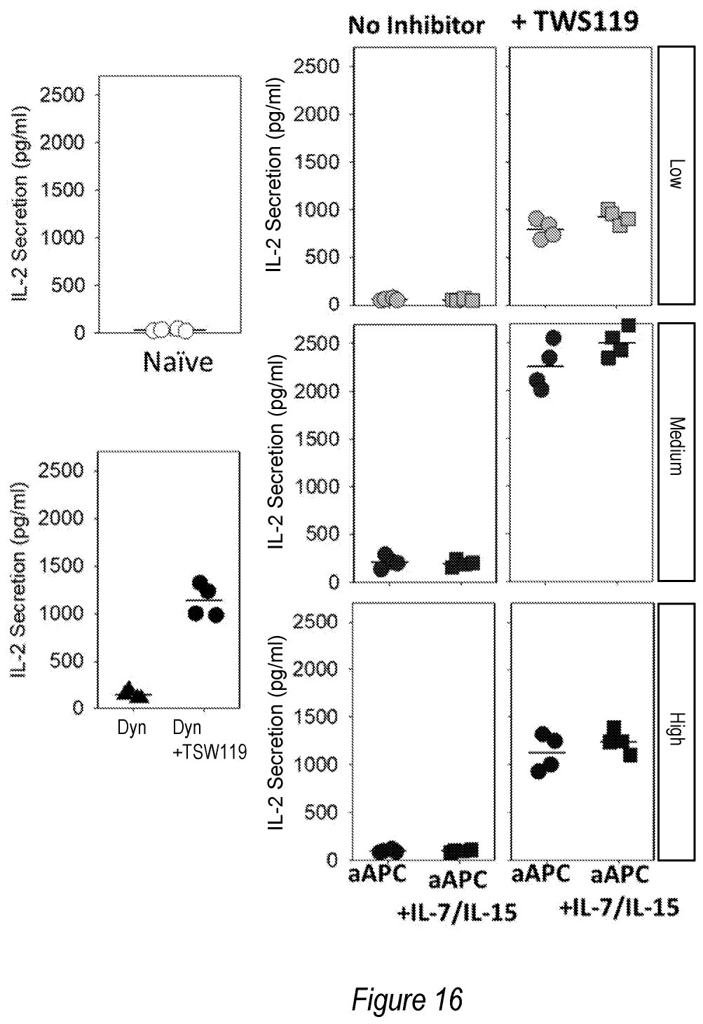

FIG. 16 shows Intracellular expression of IL-2 was used as cellular markers to quantify induced TSCMs. Naïve T cells were co-cultured with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of anti-CD3/anti-CD28 antibodies conjugated on surface of microparticles.

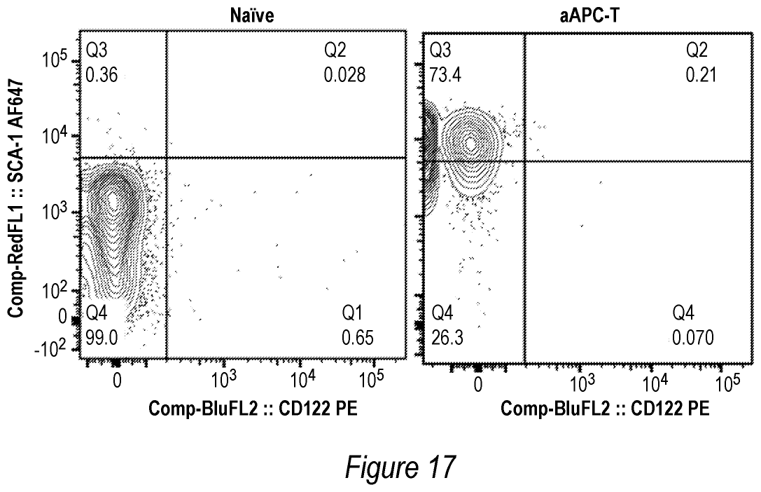

FIG. 17 depicts flow cytometry showing the difference between population of SCA-1+(CD62L+CD44− CD8+) T cells in naïve cells vs. induced TSCMs.

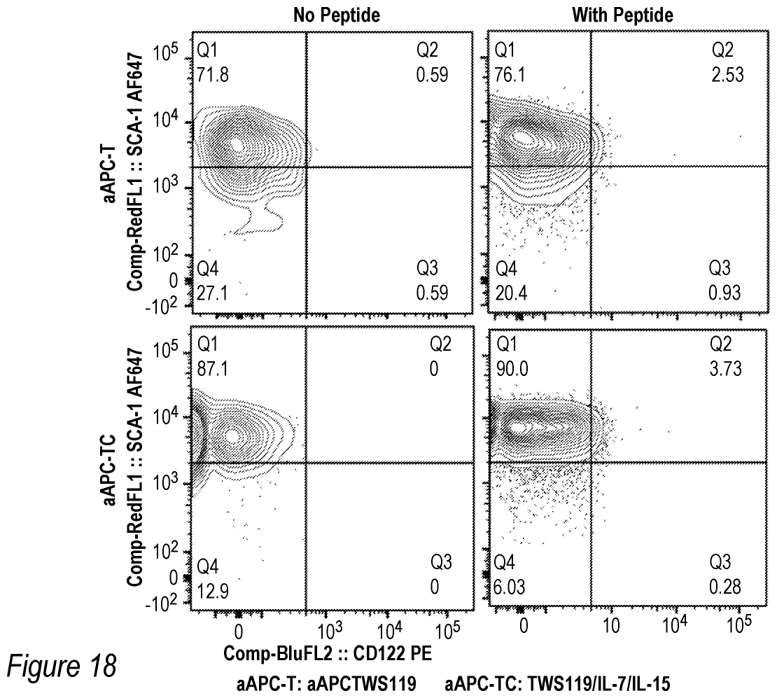

FIG. 18 depicts Flow cytometry showing the difference between population of SCA-1+CD122+ (CD62L+CD44−CD8+) T cells after being treated with aAPCs contained either TWS118 (aAPC-T) or TWS118+IL-7/IL-15 (aAPC-TC) for four days. In order to induce specificity against SARS-CoV-2 (Severe Acute Respiratory Syndrome-related coronavirus 2), the aAPCs were added to solenocyte while loaded with SARS-CoV-2 Spike Glycoprotein peptide pool. This pool includes 316 peptides (Genscript) derived from a peptide scan (15mers with 11 aa overlap) through the entire Spike glycoprotein (Protein ID: P0DTC2) of SARS-CoV-2. Antigen-specific TSCMs can be identified in SCA-1+CD122+ quadrant.

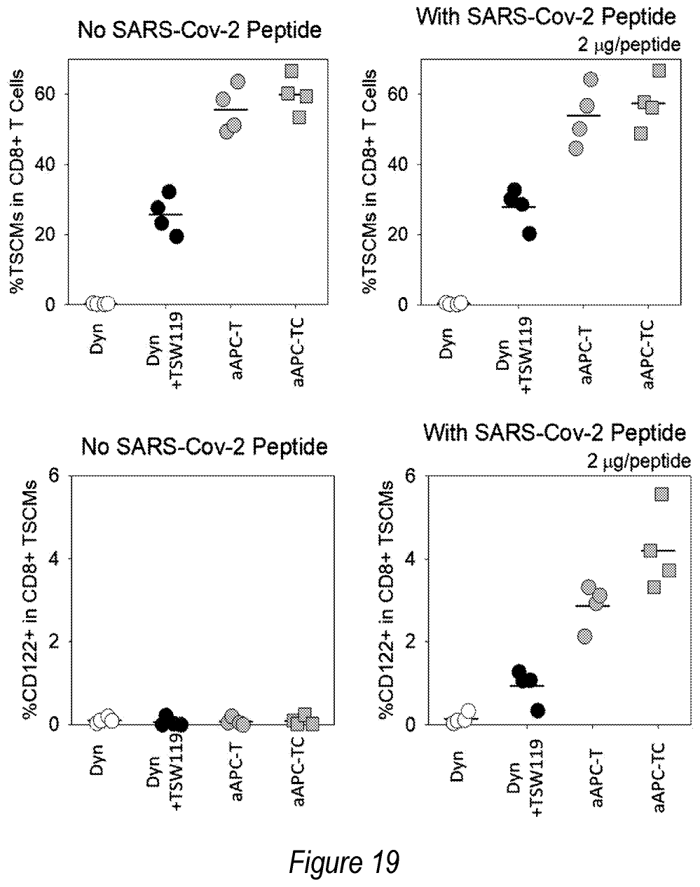

FIG. 19 shows the quantification of flow cytometry results showing the formation of polyclonal, SCA-1+, (Top panels) and SARS-CoV-2 specific, SCA-1+CD122+, (Lower panels) TMSCs. Naïve T cells being treated with aAPCs contained either TWS118 (aAPC-T) or TWS118+IL-7/IL-15 (aAPC-TC) for four days. In order to induce specificity against SARS-CoV-2, the aAPCs were added to solenocyte while loaded with SARS-CoV-2 Spike Glycoprotein peptide pool. This pool includes 316 peptides (Genscript) derived from a peptide scan (15mers with 11 aa overlap) through the entire Spike glycoprotein (Protein ID: P0DTC2) of SARS-CoV-2.

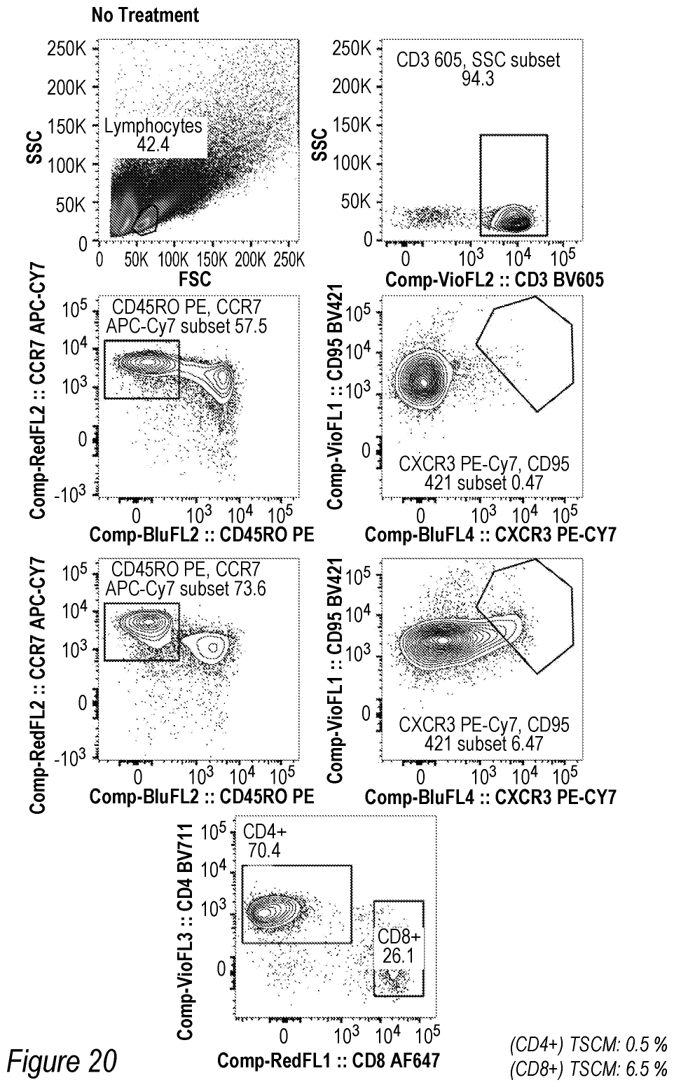

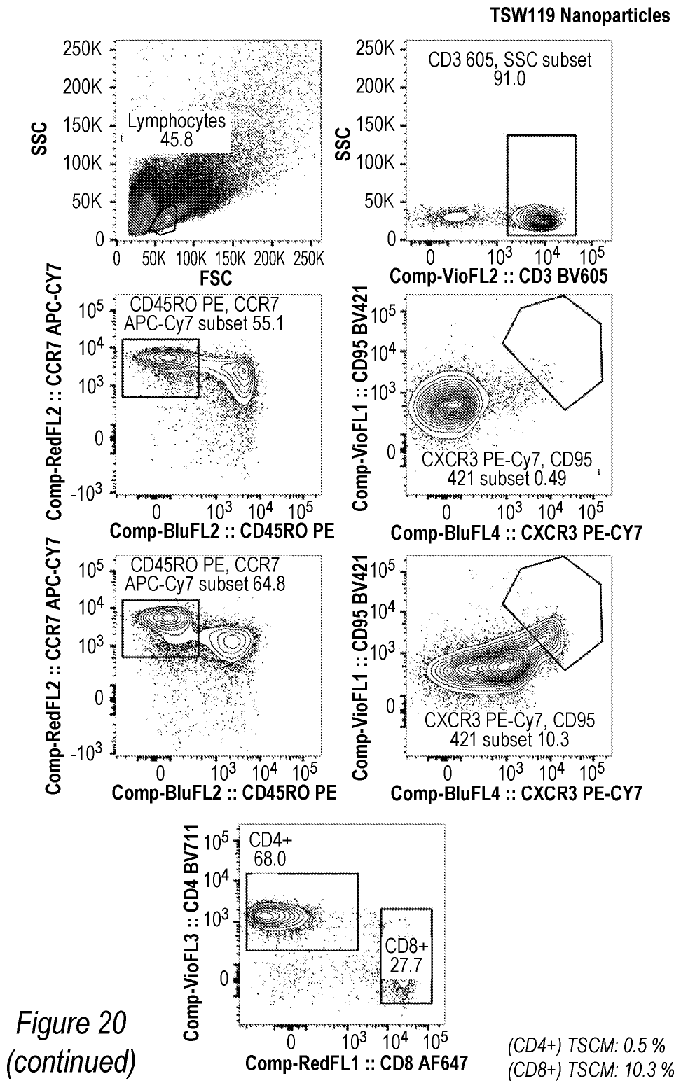

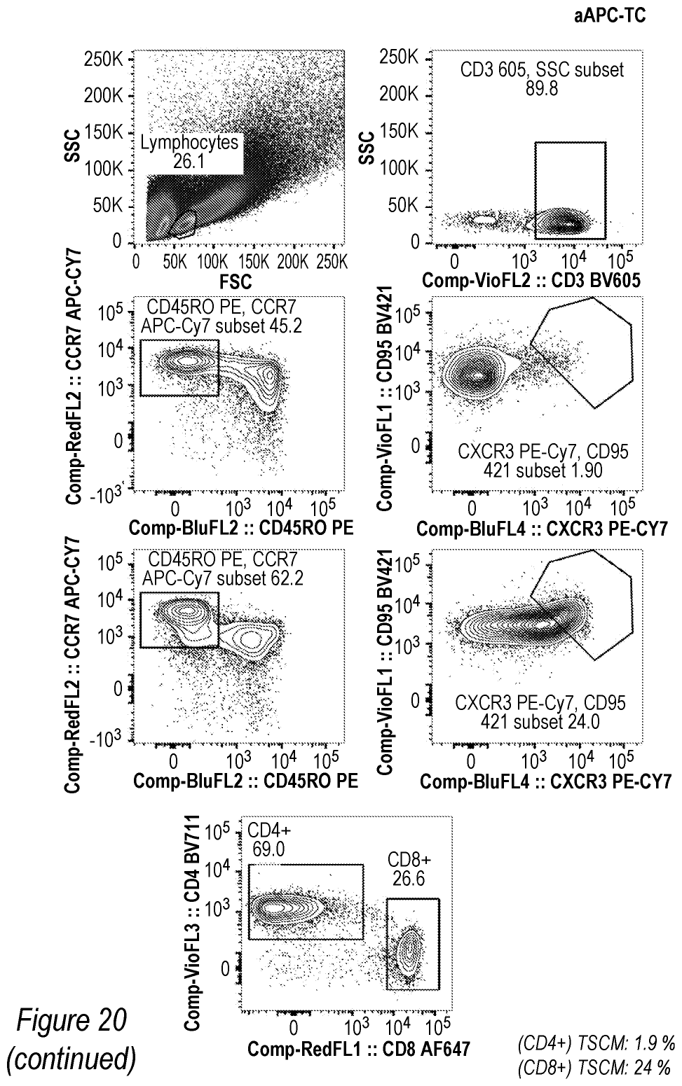

FIG. 20 depicts identification of TSCM cells in human blood. Plots show the gating strategy to identify CD95+ CXCR3+ TSCM cells in CD4+ and CD8+ T cells. Flow cytometry analysis of human PBMCs without any treatment or after being treated with aAPC-TC. Numbers indicate the percentage of cells in the indicated gates. T cell subsets were defined as follows: TN cells: CD3+CD8+(or CD4+) CD45RO−CCR7+CXCR3−CD95− and TSCM cells: CD3+CD8+(or CD4+)CD45RO−CCR7+CXCR3+CD95+.

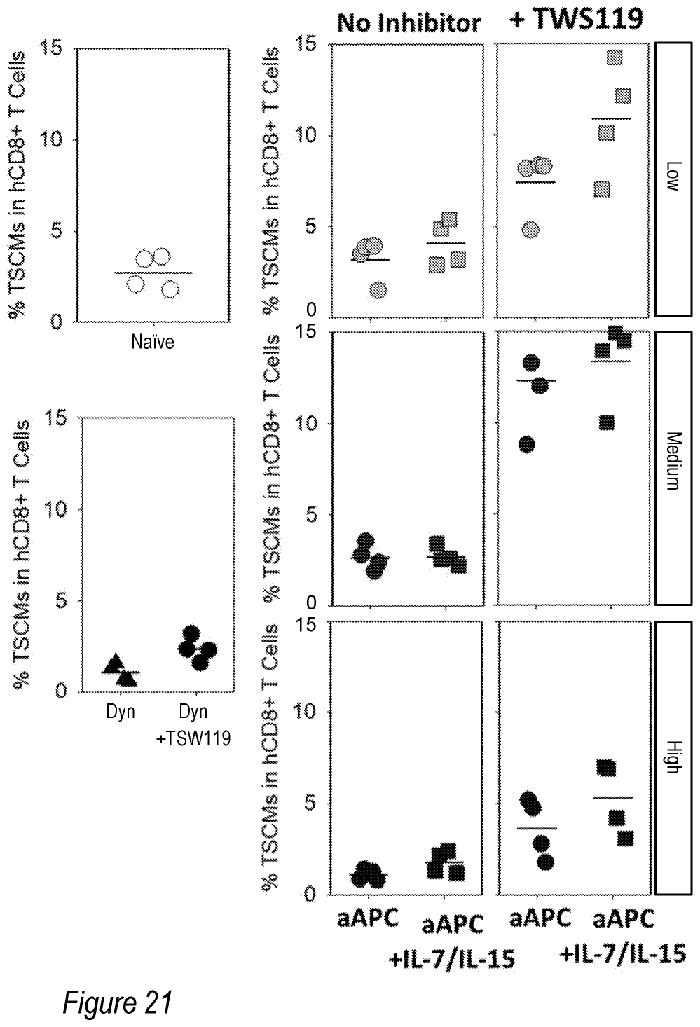

FIG. 21 shows quantification of TMSCs in human CD8+ T cells based on surface markers after four days of co-culturing with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of human anti-CD3/anti-CD28 antibodies conjugated on surface of microparticles.

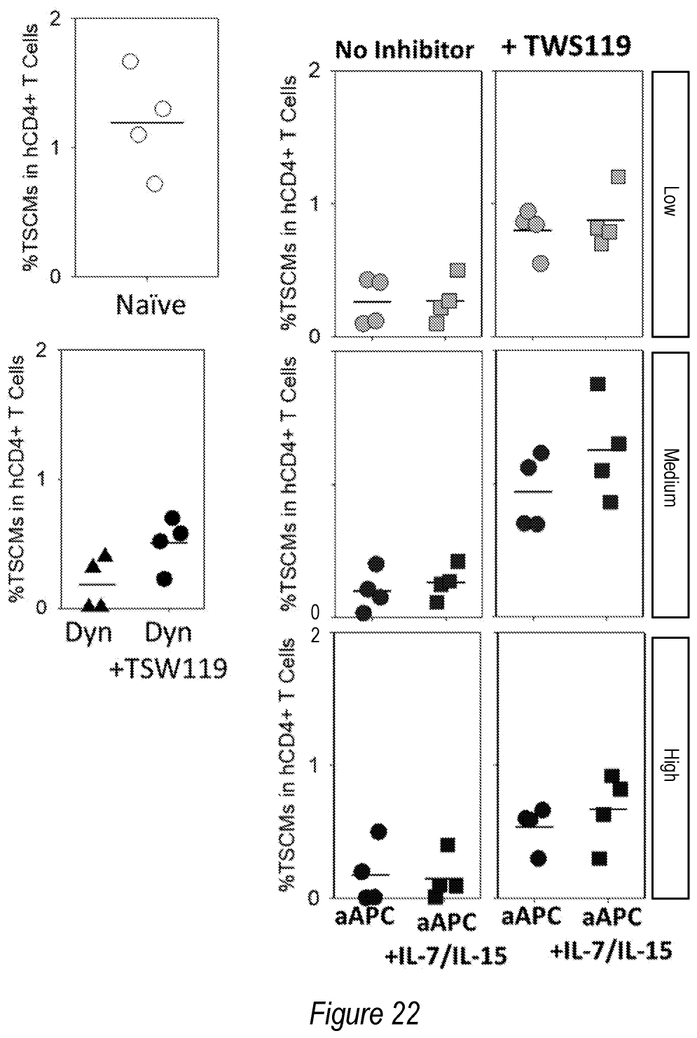

FIG. 22 depicts quantification of TMSCs in human CD4+ T cells based on surface markers after four days of co-culturing with aAPCs with and without TWS119 inhibitor and IL-7/IL-15 cytokines. Soluble addition of TWS119 inhibitor and IL-7/IL-15 cytokines in presence of commercially available Dynabeads. aAPCs at three different signal strengths (low, medium, and high) based on density of human anti-CD3/anti-CD28 antibodies conjugated on surface of microparticles.

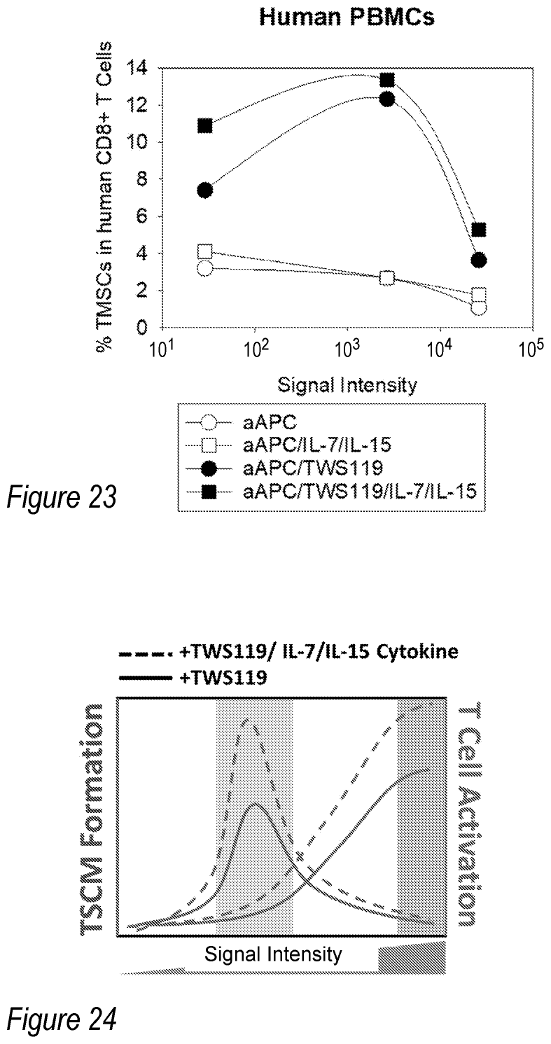

FIG. 23 is a plot showing how change in signal intensity delivered by aAPCs can tune the formation of TMSCs in human CD8+ T cells. Presence of TWS119 is essential for such an induction while co-delivery of IL-7/IL-15 can improve the efficiency and sustain the effect.

FIG. 24 is a schematic drawing showing how change in signal intensity delivered by aAPCs can tune the activation of human T cells (plots in red) or modulate formation of TSCMs. Presence of TWS119 is essential for differentiation of naïve T cells toward TSCMs while co-delivery of IL-7/IL-15 can improve the efficiency and sustain the effects.

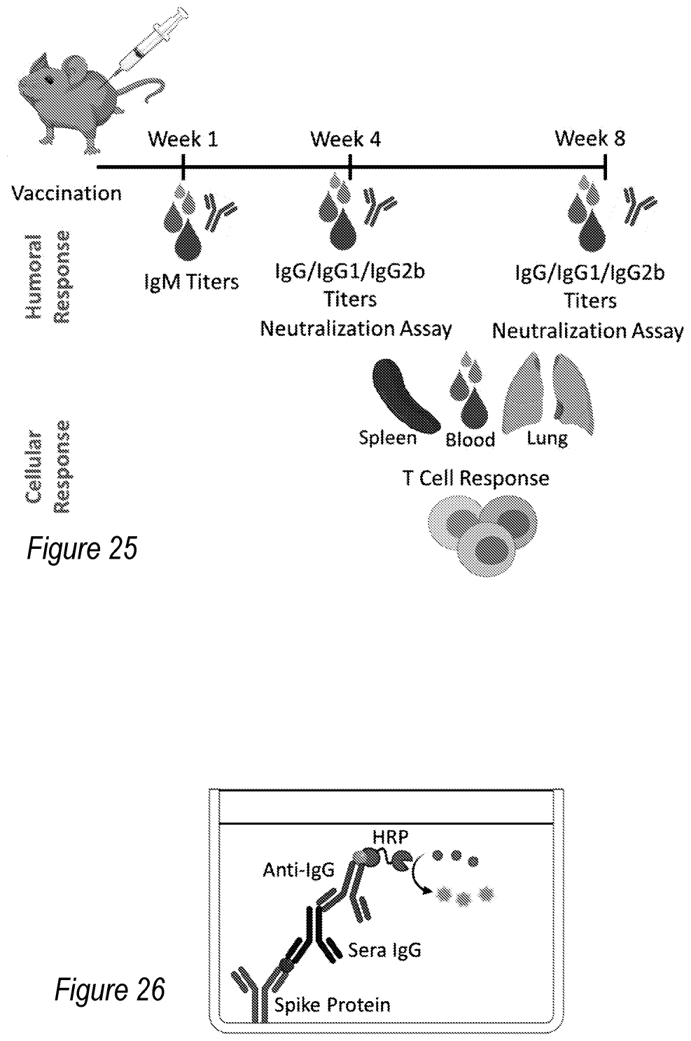

FIG. 25 shows vaccination timeline in experimental animal. Here wild-type (C57BL/6) mice were used for single dose subdermal vaccination. Blood collected from vaccinated animal at 7-day timepoint used for SARS-CoV-2 IgM detection. Blood collected at week 4 and week 8, were used to measure SARS-CoV-2 IgG, IgG1, and IgG2b titers. Lung and spleen tissues were extracted and digested into single cell to analyze cellular response after re-stimulation with r SARS-CoV-2 Spike peptides.

FIG. 26 shows the ELISA method was used to detect IgM, IgG and IgG subunits (IgG1 and IgG2b).

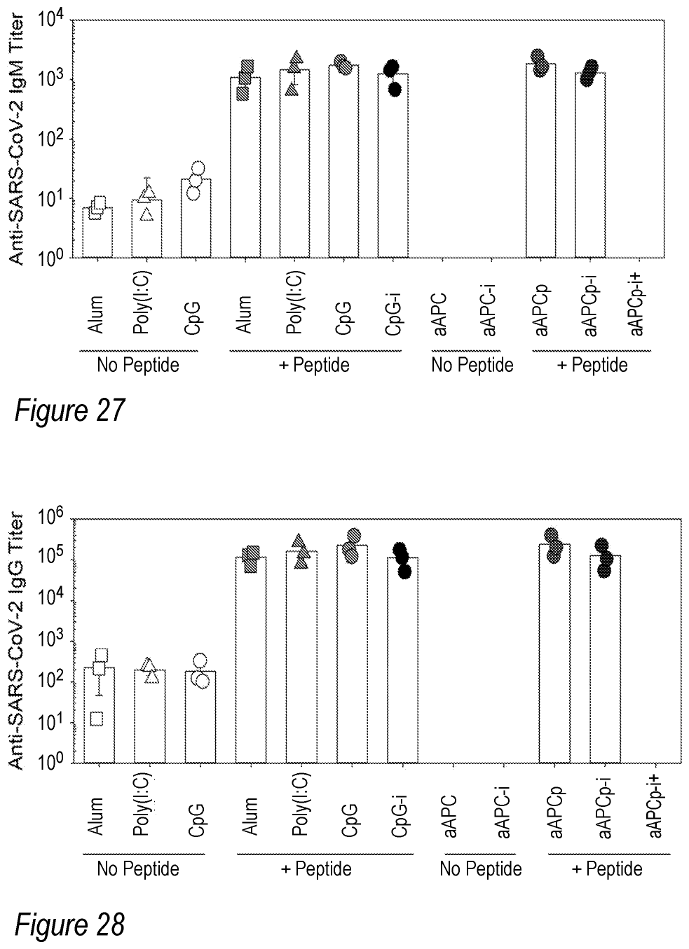

FIG. 27 depicts vaccination with aAPCs elicits potent SARS-CoV-2 Spike humoral response in C57BL/6J. Mice were immunized on days 0 with indicated formulations and humoral responses analyzed on day 7 by ELISA for IgM.

FIG. 28 depicts vaccination with aAPCs elicits SARS-CoV-2 Spike humoral response in C57BL/6J. Mice were immunized on days 0 with indicated formulations and humoral responses analyzed on day 28 by ELISA for IgG.

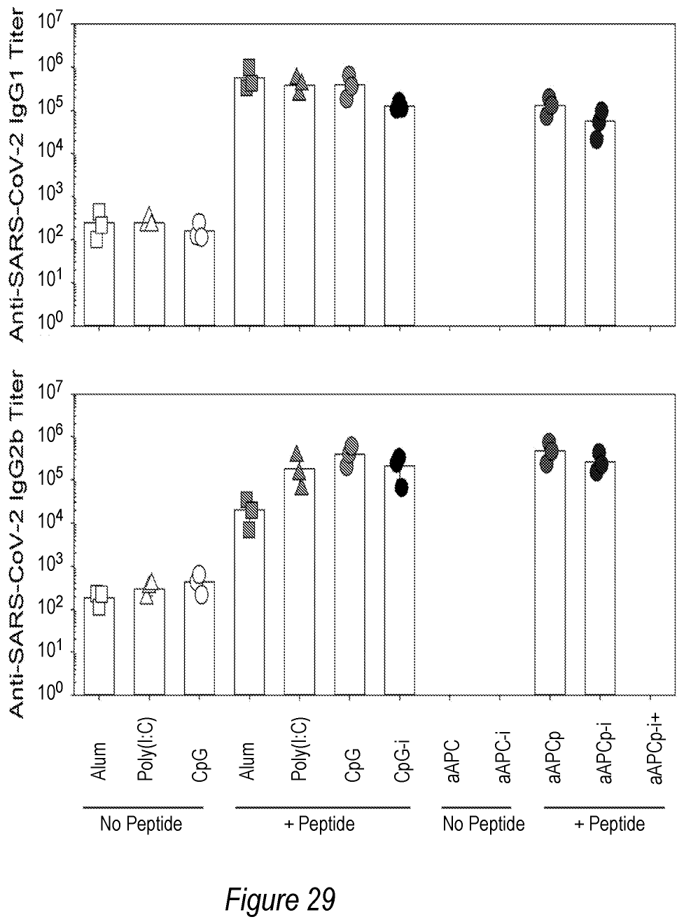

FIG. 29 depicts vaccination with aAPCs elicits SARS-CoV-2 Spike humoral response in C57BL/6J. Mice were immunized on days 0 with indicated formulations and humoral responses analyzed on day 28 by ELISA for IgG1 (Th2) and IgG2b (Th1).

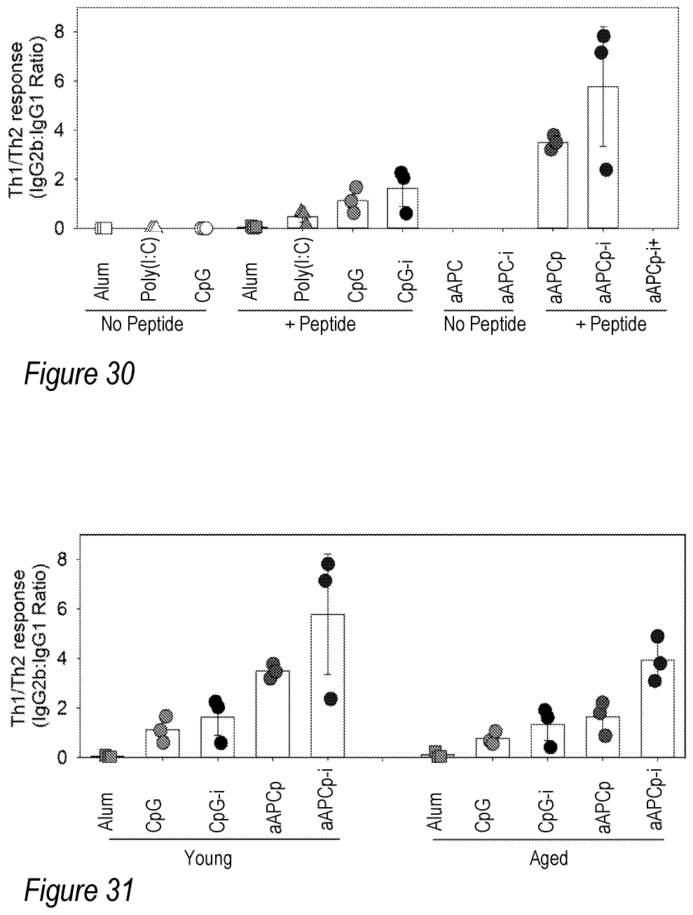

FIG. 30 depicts vaccination with aAPCs elicits potent Th1 response against SARS-CoV-2 Spike in C57BL/6J. Mice were immunized on days 0 with indicated formulations and humoral responses analyzed on day 28 by ELISA for IgG1 (Th2) and IgG2b (Th1). The ratio of IgG2b to IgG1 titer indicated a strong bias towards Th1 for aAPCp immunized animals specially when TWS119 inhibitor was presented (aAPCp-i). Soluble adjuvants Alum, poly(I:C), and CpG produced a balanced Th1/Th2 profile or Th2-dominant response while aAPCs formulations due to providing sustained release of their cargos as well as providing active engagement with local immune cells can switch the response toward Th1.

FIG. 31 depicts vaccination with aAPCs elicits potent Th1 response against SARS-CoV-2 Spike in young (3-month-old) vs. aged (13-month-old) C57BL/6J mice. Mice were immunized on days 0 with indicated formulations and humoral responses analyzed on day 28 by ELISA for IgG1 (Th2) and IgG2b (Th1). The ratio of IgG2b to IgG1 titer indicated a strong bias towards Th1 for aAPCp immunized animals specially when TWS119 inhibitor was presented (aAPCp-i). Soluble adjuvants Alum, poly(I:C), and CpG produced a balanced Th1/Th2 profile or Th2-dominant response while aAPCs formulations due to providing sustained release of their cargos as well as providing active engagement with local immune cells can switch the response toward Th1. Similar trend can be seen in aged animals.

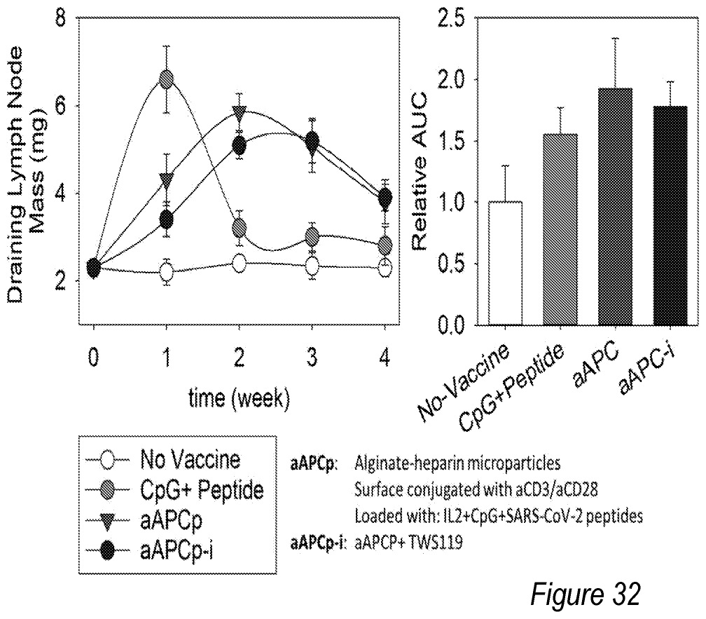

FIG. 32 shows the change in mass of vaccine draining lymph node is evaluated. Successful vaccination can result in accumulation and expansion of antigen-specific immune cells in draining lymph node close to vaccination site. Mean±SD (n=3).

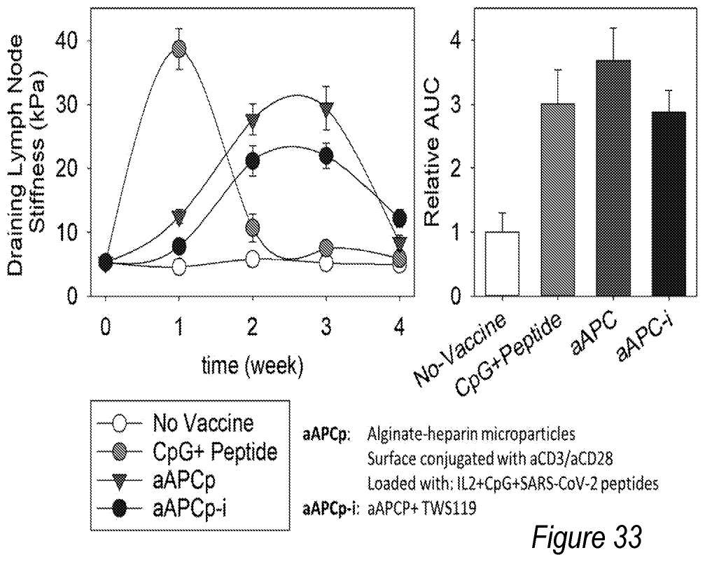

FIG. 33 shows the change in mechanical stiffness of vaccine draining lymph node is evaluated. Successful vaccination can result in accumulation and expansion of antigen-specific immune cells in draining lymph node close to vaccination site. Mean±SD (n=3).

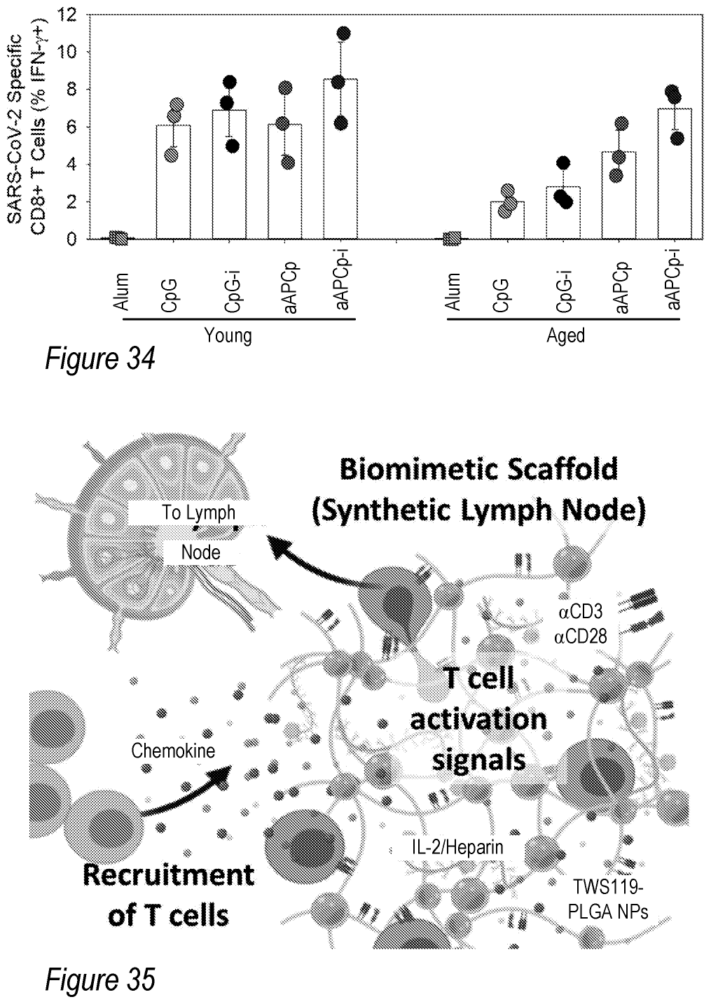

FIG. 34 depicts vaccination with aAPCs elicits enhanced SARS-CoV-2 Spike-specific T cell responses in C57BL/6J. Mice were immunized on day 0 and T cell responses analyzed on day 28. peripheral blood cells collected at week 4 and were analyzed by flow cytometry after being stimulated with SARS-CoV-2 Spike peptide pool. This pool includes 316 peptides (Genscript) derived from a peptide scan (15mers with 11 aa overlap) through the entire Spike glycoprotein (Protein ID: P0DTC2) of SARS-CoV-2. This stimulation can trigger intracellular cytokine production to detect antigen-specific T cell responses. Shown are frequencies of IFNγ, T cells among peripheral blood CD8+ T cells.

FIG. 35 shows the mechanism of action of the 3D scaffolds in training tissue resident immune cells to induce robust and prolonged immunity against SARS-CoV-2 infection.

### Example 8. Induction of T Memory Stem Cells In Vivo to Boost Vaccine Immunity

### Materials and Methods

Unless noted otherwise, all chemicals were purchased from Sigma-Aldrich, Inc. (St. Louis, MO). All glassware was cleaned overnight using concentrated sulfuric acid and then thoroughly rinsed with Milli-Q water. All the other cell culture reagents, solutions, and dishes were obtained from Thermo Fisher Scientific (Waltham, MA), except as indicated otherwise. Interlukine-2 (IL-2) was kindly provided by BRB Preclinical Repository (NCI, NIH, Frederick, MD). All in vitro experiments were conducted in accordance with UCLA's institutional policy. Human samples were deidentified blood samples from commercial resources. Animal studies were performed following the approval by the Institutional Animal Care and Use Committee.

TWS119 Nanoparticles. Poly(lactic-co-glycolic) acid (PLGA) nanoparticles (NPs) were prepared to control presentation of 4,6-disubstituted pyrrolopyrimidine (also called TWS119, Cayman Chemicals). PLGA-TWS119 nanoparticles were prepared using a nanoprecipitation method as previously reported. Briefly, 200 mg carboxylic acid-terminated PLGA (Resomer XX; 50:50) was dissolved in 5 ml dichloromethane and stirred with TWS119 (dissolved in DMSO) prior to being sonicated into 1 wt/v % poly (vinyl alcohol) (PVA) aqueous solution (50 ml) by probe sonicator for 2 minutes. The resulted emulsion was then added to 100 ml of 0.5 wt/v % PVA solution and stirred at 40° C. for 4 hours to accelerate evaporation dichloromethane. The solution was then centrifuged at 1500×g for 5 minutes to remove macro-size aggregates. The supernatant was ultracentrifuged and washed three times at 21,000×g for 20 minutes to isolate nanoparticles. The resulting nanoparticle solution was flash frozen in liquid nitrogen and lyophilized for 3 days prior to use. Hydrodynamic diameter and surface charge of formed PLGA nanoparticles were studied using dynamic light scattering (DLS) measurements (Zetasizer 3000HS, Malvern Instruments Ltd., Worcestershire, UK) in backscattering mode at 173° for the diluted suspensions in water.

Preparation and Characterization of Artificial Antigen Presenting Cells (aAPCs). Alginate-based microparticles were fabricated as reported before. The ultrapure and GMP-grade alginate (PRONOVA UP MVG, NovaMatrix) was used in this study. Microparticles were formed using hydrophobic glass microfluidic droplet junction chips (Dolomit, Charlestown, MA) with 100 μm of inner channel diameter. Dual, physical (ionic) and chemical (covalent), crosslinking was utilized to form microparticles with variety of sizes and stiffnesses. Briefly, a mixture of alginate (or Alginate-heparin) solution (1% w/v) and four-arm polyethylene glycol hydrazide (5 kDa, Creative PEG work, Chapel Hill, NC) (5 mM) was used as the aqueous phase. Mineral oil modified with Span 80 (10 wt %) surfactant was used as the continuous phase. Size and rate of formed droplets were controlled by tuning the flow rates using two syringe pumps (Fusion 200, Chemyx, Stafford, TX) for aqueous and oil flows independently. Stability and quality of formed droplets were tracked using a Leica DMIL inverted fluorescence microscope. Formed droplets were collected in calcium ions (100 mM CaCl₂) bath and incubated at 37° C. for 30 minutes for post-formation crosslinking prior to being washed three times with NaCl solution (20 mM) and concentrated (20,000 rpm for 5 minutes) before being incubated in a hydroxybenzotriazole (HOBt) and 1-ethyl-3-(3-dimethylaminopropyl) carbodiimide (EDC) solution for 2 hours to allow the chemical crosslinking. Microparticles were washed, concentrated, and kept at −20° C. prior use. For in vitro studies, alginate microparticles were modified with super paramagnetic iron oxide nanoparticles (SPION; 50 nm, carboxylated, Chemicell GmbH, Berlin, Germany) for ease of separation from the cultured cells. Alginate/SPION solutions was probe sonicated for 2 min at 40% of intensity at 4° C. prior to being used as aqueous solution for microparticle formation.

Carbodiimide chemistry was used to conjugate anti-CD3 (2C11; Bio X Cell) and anti-CD28 (37.51; Bio X Cell) to the surface of microparticles as we reported before. Briefly, microparticles were dispersed in conjugated buffer (MES 150 mM, NaCl 250 mM, pH 6.5) and being mixed with EDC and NHS for 10 min to activate carboxylic groups of alginate matrix. The microparticles then being washed with calcium (1 mM CaCl₂) supplemented PBS twice prior to being incubated with three different concentrations of CD3/CD28 antibodies at 4° C. overnight. The antibodies-functionalized microparticles were then washed and stored at −20° C. prior to further use. Unreacted activated functional groups were quenched using Tris buffer (100 mM, pH 8).

Microparticles were prepared at three different antibody densities to study effect of density of activation signals on T cells fate. The maximum (High) antibody density is similar to the one used for conventional plate-bound stimulation (0.01 μg/mL). Dilutions of 10- and 100-fold were made as medium and low conjugation densities. When various size of microparticles were used, the number of particles at different sizes were normalized to their surface area in order to provide similar total surface for T cell engagement and activation.

Quantification of the total amount of anti-CD3/CD28 antibodies presented on functionalized particles was analyzed using micro-BCA assay according to the manufacturer's protocol. Dynamic light scattering (DLS) measurements were performed using a Zetasizer. The content of magnetic nanoparticles in fabricated microparticles were estimated by measuring the iron content of the particles using Inductively Coupled Plasma-Optical Emission Spectrometry (ICP-OES) after digestion the particles with EDTA and alginate lyase.

Encapsulation of Antigens, Cytokines and Small Molecules in Micro/Nano Particles. It has been shown that presence of heparin in formulation can significantly increase the binding affinity of positively charged proteins or those with heparin-binding domains. Chemical synthesis of alginate-heparin was performed as we reported before. In the current study, selected cytokines, IL-2, Il-7, and IL-15 were mixed with alginate/alginate-heparin prior to being injected into the microfluidic platform for microparticle formation.

To minimize the efforts on peptide antigen development, the stablished SARS-CoV-2 Spike Glycoprotein peptide pool by GenScript (Piscataway, NJ) was utilized. This pool includes 316 peptides derived from a peptide scan (15-mers with 11 amino acid overlap) through the entire Spike glycoprotein (Protein ID: P0DTC2) of SARS-CoV-2. Various concentration of these peptide mixture was mixed with alginate-heparin conjugate prior to droplet formation and crosslinking.

To study the in vitro release of cytokines and peptides from microparticles, 20×10⁶ microparticles incubated in 2 ml PBS (pH 7.4; supplemented with 1 mM CaCl₂) at 37° C. At predetermined time points, 500 μl of the supernatant was collected, and replaced with an equivalent volume of PBS. The concentration of released cytokines were determined using enzyme-linked immunosorbent assay (ELISA) kits (R&D Systems) for each cytokine individually and in combination to assess the possible protein-protein interaction. Release kinetic of SARS-CoV-2 Spike Glycoprotein peptides was estimated using micro-BCA assay as mentioned before.

To form microparticles for TWS119 delivery, PLGA-TWS119 nanoparticles were mixed with alginate/alginate-heparin prior to forming microparticles. The concentration of released TWS119 from nanoparticles before and after loading into microparticles was determined by measuring the UV absorption of TWS119 at 315 nm.

Characterization and Sterilization of Microparticles. For stiffness characterization of microparticles, standard V-shaped gold-coated silicon nitride tipless AFM cantilevers (Bruker/Veeco, BrukerNano, Camarillo, CA) were modified with 10 μm diameter latex beads as described before. The spring constant of modified cantilevers were measured by thermal fluctuations technique to be 0.15 N/m. Measurements were performed using a Veeco AFM II Dimension 3100 (BrukerNano) instrument in liquid mode after immobilizing microparticles. The force displacement curves were recorded at a frequency of 1 Hz with the vertical ramp size of 5 μm.

Scanning electron microscopy (SEM) images of the cells and microparticles were taken to see the morphology of microparticles as well as their interaction with T cells. Here cell-microparticles were fixed with 2.5% glutaraldehyde, followed by post-fixation in osmium tetroxide prior to serial dehydration in increasing concentrations of ethanol (25, 50, 75, 90, and 100%) for 15 min each, and iridium sputtering. Samples were sputtered with iridium (South Bay Technology Ion Beam Sputtering) prior to imaging with a ZEISS Supra 40VP scanning electron microscope (Carl Zeiss Microscopy GmbH).

To sterilize the fabricated microparticles before in vitro or in vivo functional assays, X-ray irradiation (Gulmay Medical RS320 X-ray unit) was employed as per ISO 11137-2:2013 recommended protocols. A dose of 25 kGy (2.5 Mrads) was used for sterilization. Our results indicated no significant (p>0.05) change in biological activity of particles following the X-ray irradiation.

T Cell Culture and Characterization. Pathogen-free C57/B6 mice (Jackson Labs) were used to isolate muse T cells. T cell-culture media is RPMI supplemented with 10% heat-inactivated FBS, 1% penicillin/streptomycin, 1% sodium pyruvate, 1% HEPES buffer, 0.1% μM 2-mercaptoethanol. CD4⁺/CD8⁺ T cells were purified from mice spleens and lymph nodes using negative selection enrichment kits (Stem Cell Technologies) as reported before. Peripheral blood mononuclear cells (PBMCs) from healthy human donors were used as cell source for naïve human T cell source in this study to generate human T memory stem cells in vitro. PBMCs were isolated by Ficoll-Hypaque gradient separation (Lymphoprep, Fresenius) and enriched for CD3⁺ cells with the human CD3 MicroBeads Isolation Kit (Miltenyi Biotec) following the manufacturer's instruction. Direct co-culturing with was used for assessing the effectiveness of the designed formulations on the polarization, activation, maturation, and differentiation of primary naïve T cells. After 48 hours of stimulation of 10⁵ of naïve T cells, the activation markers were tested, and cells were cultured for 14 additional days in order to evaluate the ex vivo expansion and assess the sternness of treated cells. The change in T cell activation, differentiation, and formation of TMSCs were tested at using flow cytometry by checking the surface markers including CD4, CD8, CD45RO⁻, CD95⁺, CXCR3⁺, and CCR7⁺ for human T cells and CD4, CD8, CD62L⁺, CD44⁻, CD122⁺, and Sca-1⁺ for murine T cells while expressing intracellular IFN-γ cytokine. For intracellular cytokine staining (ICCS), cells were fixed with cold 4% paraformaldehyde (PFA) for 20 minutes at room temperature and then permeabilized with 0.5% saponin for 10 min before blocking with phosphate buffered saline (PBS)+1% bovine serum albumin (BSA)+5% donkey serum and staining with appropriate antibodies at 1:100 dilution. Cells were washed and analyzed by flow cytometry as above. The following antibodies were used for ICCS from Biolegend: IL-2 (clone JES6-5H3, 503808); TNFα (clone MP6-XT22, 506313), IFN-γ (clone XMG1.2, 505814).

In addition to flow cytometry, the expression of LEF1, and TCF7 was assayed by Quantitative PCR (qPCR) analysis. Total RNA was isolated using TRIzol reagent. RNA was reverse transcribed, and single-stranded complementary DNA (cDNA) was synthesized using the SuperScript III cDNA Synthesis Kit. Relative gene expression was calculated using the 2^{-ΔΔCt} method, with normalization to the Cₜ of the housekeeping gene housekeeping gene encoding β-actin (Actb).

In Vivo Vaccination. The efficacy of designed vaccine formulations was tested by using murine model as reported before for SARS-CoV and SARS-CoV-2. All animal experiments were conducted in accordance with UCLA's institutional policy on humane and ethical treatment of animals following protocols approval by the Animal Research Committee (Protocol #ARC-2020-131). Immunologically mature (6-10 weeks-old) and aged (10-13 month-old) wild type C57/B6 mice were used for this study. Male C57BL/6 mice (five animals per group) were subjected to subcutaneous injection into the abdomen of anesthetized mice. Mice were injected with 50 μl of PBS containing designed formulations. All formulations will deliver an adjuvant (e.g., 5 nmol of CpG) to induce immunogenicity at the site of vaccination. Three adjuvants were used: alum, Poly(I:C), and CpG to optimize the formulation.

Analysis of Humoral Response. In order to test the effectiveness of the proposed approach in inducing SARS-CoV-2 antibody production, mice were bled from the saphenous vein at week 0, 1, 4, and 8, and sacrificed by the end of week 8. Serum samples can be evaluated by ELISA for SARS-CoV-2-S1 antibody production. ELISAs can be performed to determine sera antibody binding titers according to published protocols. Briefly, ELISA plates were coated with 200 ng/well CoV2 RBD protein (GenScript; Cat: Z03483) overnight at 4° C. Plates were pre-blocked with 2% BSA for 2 hours at 25° C. Serially diluted mouse sera were transferred to the ELISA plates and incubated for 2 hours. Plates were washed with washing buffer (BioLegend) 4 times and then incubated for 1 hour with a secondary antibodies of horse radish peroxidase (HRP)-conjugated rabbit anti-mouse IgM, HRP-rabbit anti-mouse IgG (Fcγ), HRP-goat anti-mouse IgG1 and IgG2b (Jackson ImmunoResearch) at dilution of 1:2000. Plates were again washed with washing buffer 4 times; after which the plates were developed with 3,3′,5,5′-tetramethytlbenzidine for 15 min, and the reaction was stopped with sulfuric acid (1N). The absorbance at 450 nm was measured by an ELISA plate reader. Titers were determined at an absorbance cutoff of 0.5 OD. Anti-SARS-CoV-2 IgG ELISA using commercial kits (Sino Biological) can be performed according to manufacturer's protocol as reported before.

SARS-CoV-2 Surrogate Neutralization Assay. For the surrogate neutralization assay the cPass kit (GenScript) was used according to manufacturer's instructions. Here, serially diluted sera were incubated with SARS-CoV-2 Spike RBD-HRP and added to ACE2 precoated plates and ELISA assay was performed as mentioned above.

Pseudoviral Neutralization Assay. SARS-CoV-2 pseudotyped lentivirus (pSARS-CoV-2; Luc-eGFP; BPS Bioscience, San Diego, CA) was used in a neutralization assay using ACE2 expressing cells in vitro, as previously described. The selected pseudotyped Lentivirus was produced by replacing the VSV-G fusion glycoprotein with SARS-CoV-2 Spike protein (Genbank #QHD43416.1) as a surrogate viral envelope protein. These pseudovirions also contain a firefly luciferase and eGFP cassette (Luc-P2A-eGFP) driven by a CMV promoter. The luciferase and eGFP are co-expressed under the CMV promoter in the transduced cells. Therefore, the Spike-mediated entry into the target cell can be conveniently measured via luciferase reporter activity or eGFP expression in a Biosafety Level 2 facility. To test neutralizing antibody responses, serum from mice (immunized and control) were preincubated with pseudotyped lentivirus at various dilutions. After incubation for 1 h at 37° C., the mixture was added to ACE2/HEK293 cell (#79951; BPS Bioscience, San Diego, CA) to detect viral infectivity. Media was changed the following day and 48 hours after infection, eGFP signal in infected cells was determined by plate reader and flow cytometry.

Analysis of Cellular Immune Response. The population of cytokine (e.g., IFNγ, TNFα, and IL-2) producing CD8⁺ or CD4⁺ T cells extracted from peripheral blood and spleen from C57BL/6J mice on weeks 4 and 8 post-immunization were assessed using flow cytometry using ICCS assay. Here, after isolation of T cells, they were activated overnight with SARS-CoV-2 Spike glycoprotein overlapping peptides at 5 μg/peptide per ml (consisting of 315 peptides, derived from a peptide scan [15-mers with 11 amino acid overlap] through Spike glycoprotein of SARS-CoV-2) (GenScript). Phorbol Myristate Acetate (PMA, 50 ng/mL) and ionomycin (1 μM) were used as positive controls, and complete medium only as the negative control. Following the stimulation, cells were stained with the anti-mouse CD3/CD4/CD8a/IL-2/IFNγ/TNFα (Biolegend) antibodies. Sample acquisition was performed on Cytek Dxp10 (3 laser, 10 color) digital flow cytometer and data analyzed with FlowJo V10 software (TreeStar). Culture supernatants were also harvested and used for ELISA assay to assess production of Th1/Th2 cytokines.

Study the Formation of TMSCs In vivo. To analyze SARS-CoV-2-specific activated T cell, vaccine site draining lymph nodes were removed from mice 7 days after immunization, and single cell suspensions were prepared in RPMI 1640 containing 10% fetal calf serum (FCS). Following (2×) washing, cells were analyzed by flow cytometry to study T cell activation and differentiation as well as analyzing TMSC population based on the expression of surface markers as well as intracellular cytokine secretion in T cell subset, as described above.

Mechanical Stiffness of Vaccine-Draining Lymph Nodes (dLNs). LNs were extracted from mice 0, 1-, 2-, 3-, and 4-weeks post-immunization in wet state in PBS, and the mechanical property of dLNs was characterized by using an Instron mechanical apparatus under unconfined compression at a constant speed of 1 mm/min. LN dimensions were measured using a digital caliper prior to measurement. The elastic modulus, E, was calculated from the slope of the stress vs. strain curves (first 10% of strain).

Immunocompromised Mouse Model of Stem Cell Transplants. Transplantation of human bone marrow-derived mesenchymal stem cell (hBMMSC) was used to create immunocompromised mouse model. Here, wild-type C57BL/6J WT mice (6-8 weeks old) received subcutaneous injection of 4×10⁶ hBMMSCs. To induce ectopic bone formation, spray-dried hydroxyapatite microparticles with average size of 5 μm (Fluidinova, S.A.) were mixed with the hBMMSCs at 1:1 ratio prior to injection as reported before. To create mice with immunosuppression in order to save the stem cell transplants, Cyclosporine A, a clinically used immunosuppressant drug, was injected into mice at constant dose of 1 mg/kg/day until being sacrificed. In vivo bone formation at eight-week time point following hBMMSCs/hydroxyapatite injection was assessed using a micro-CT scanner (uCT SkyScan 1172; SkyScan), and volumetric data were converted to Digital Imaging and Communications in Medicine (DICOM) format which used to generate 3D and multiplanar reconstructed images to make measurements. Mice received Vax-T or soluble vaccines similar to healthy mice without immunosuppression. Humoral and cellular responses against SARS-CoV-2 were assessed in similar manner as mentioned above.

Hematology, Biochemistry, and Tissue Collection. After 7 days of vaccination, all mice were sacrificed, and blood and major organs were collected for blood analysis and organ studies. Using a standard blood-collection technique, 1000 μL blood was drawn into LTT-Lavender top EDTA anticoagulant collection tube for hematology analysis. Blood serums were also isolated using SST-serum Separator Tube containing clot activator with gel for blood chemistry analyses. Samples were analyzed by IDEXX BioAnalytics (West Sacramento, CA). Major organs from these mice were also extracted, fixed and processed, and stained with hematoxylin and eosin. Pathology was examined using a digital microscope.

Statistical Analysis. The Kruskal-Wallis rank sum test, one-way ANOVA and two-tailed Student's t-test were utilized as appropriate to analyze the data at a significance of a or p<0.05. Quantitative data were expressed as mean±standard deviation (SD). To determine the number of specimens for the proposed experiments, power analysis was conducted based on preliminary data.

### Results

Engineering Artificial Antigen Presenting Cell (aAPC) Platform to Induce Human TMSC in vitro. To investigate the capability of the drug delivery approach to induce TMSCs in vitro, aAPCs were first fabricated and characterized for T cell activation. A microfluidic device featuring a droplet generator was used to fabricate microparticles from alginate as the matrix for aAPCs. Alginate has excellent biocompatibility and controllable biodegradation and mechanical property. Degree of oxidation, molecular weight, and calcium content are tuned to control how fast the alginate-based scaffold will degrade in vitro and in vivo. Alginate-based microparticles comprising various concentrations of polymer from 0.5 to 5 wt % were generated. The flow ratios were tuned to make spherical particles with a variety of sizes ranging from 400 nm to 250 μm. The particles were crosslinked to increase mechanical stiffness, as measured by atomic force microscopy (AFM) (FIG. 42).

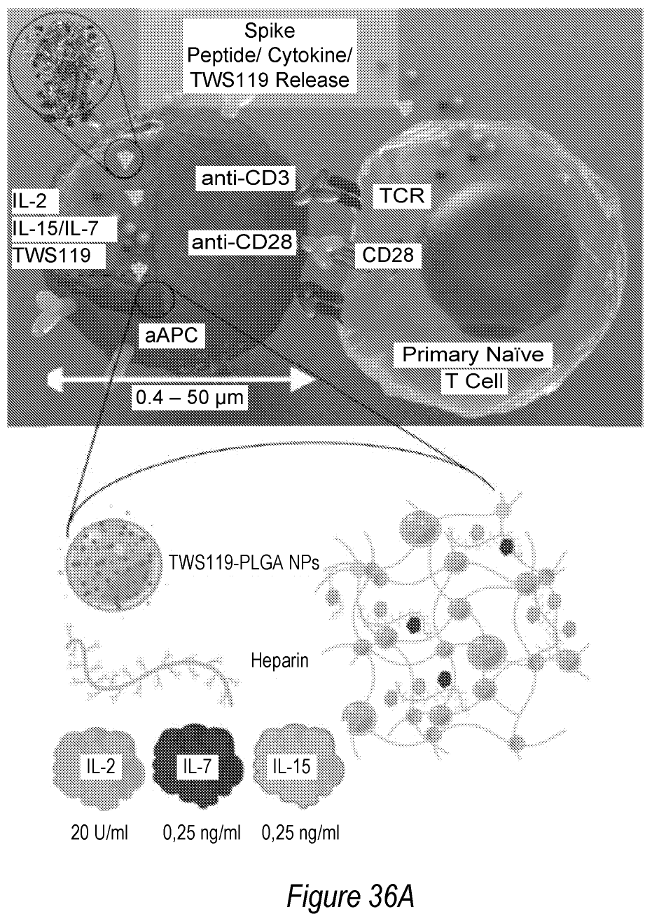

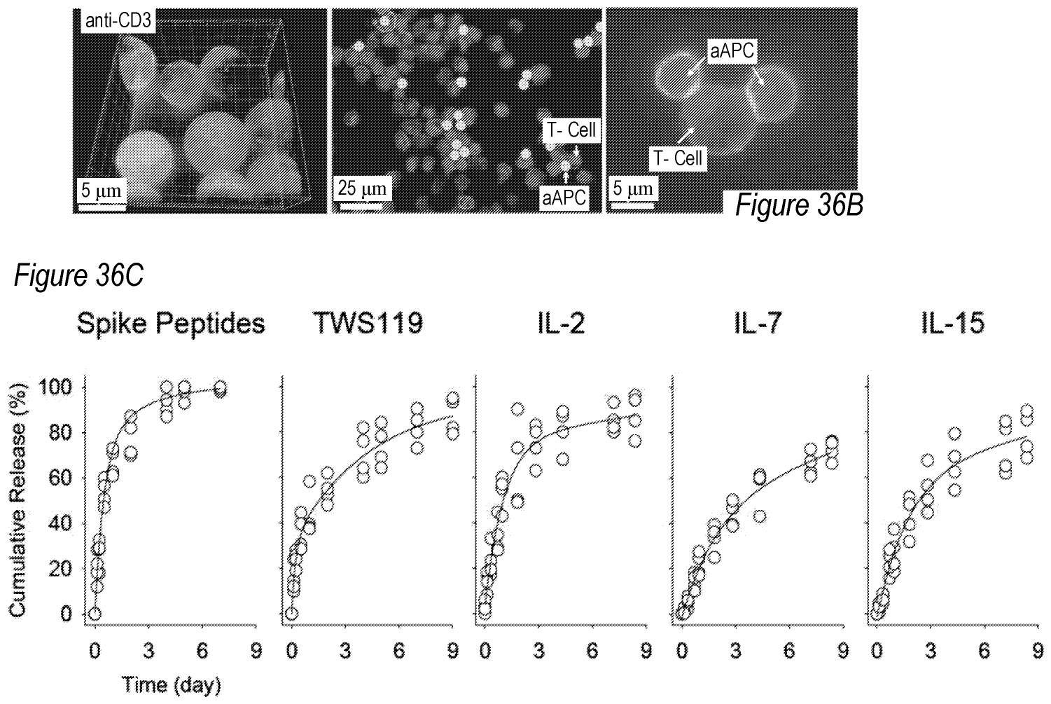

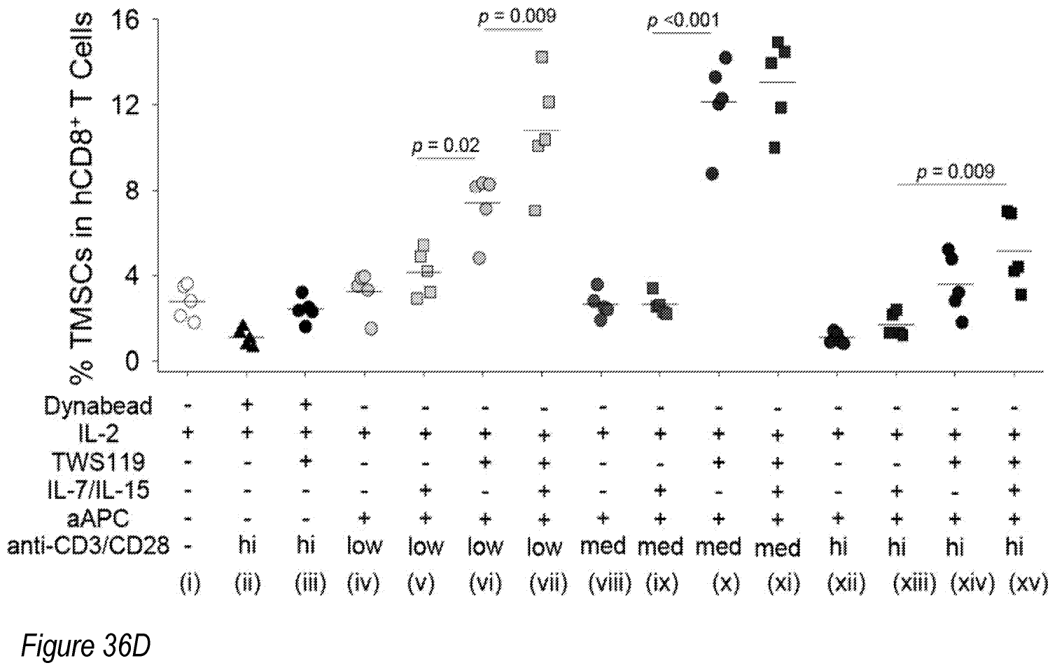

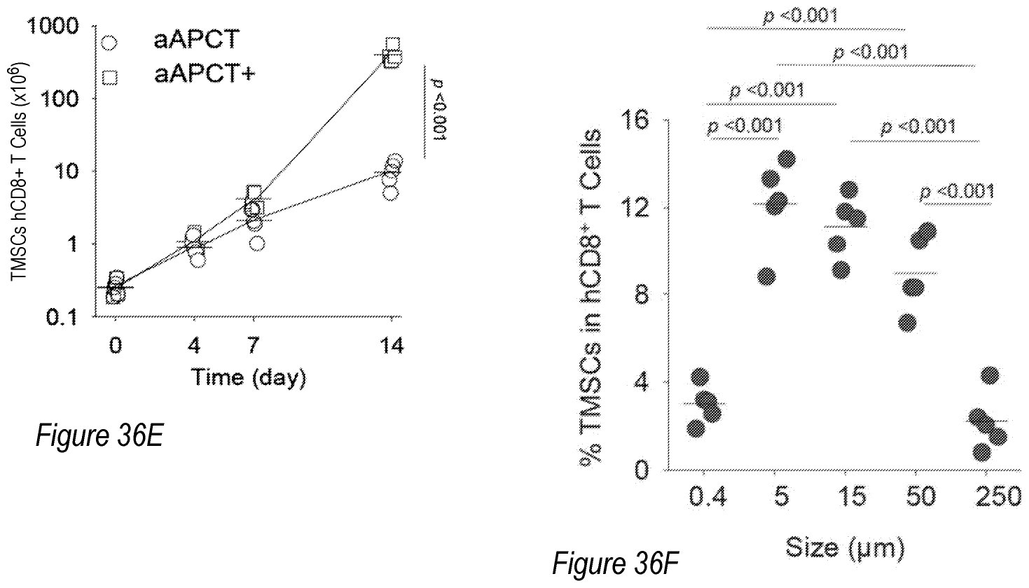

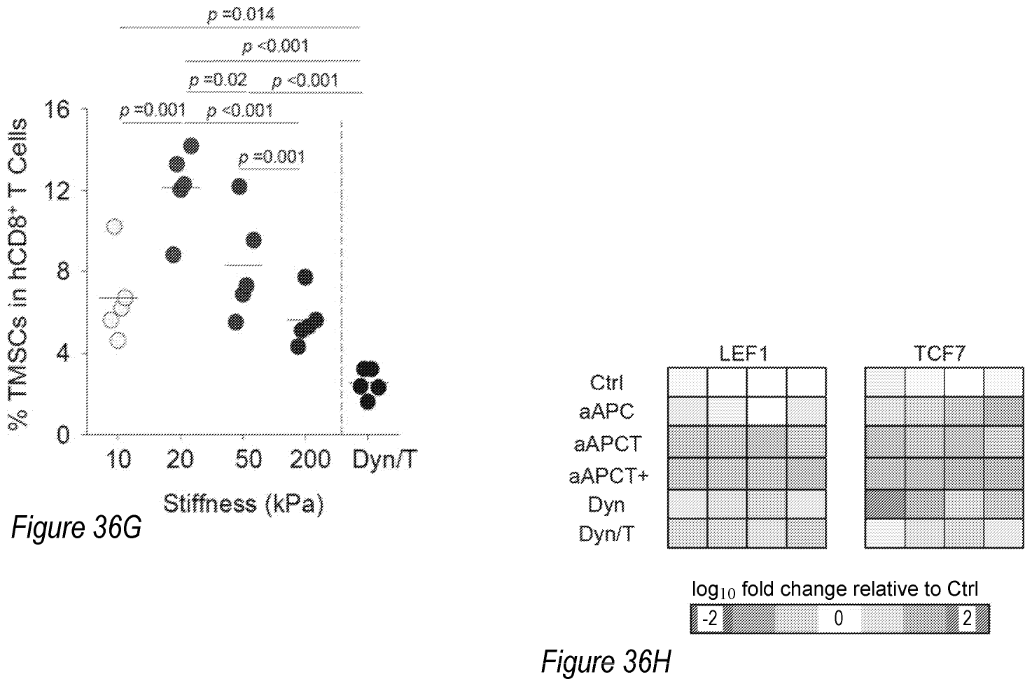

To test the efficacy of these particles in activating T cells, CD3 and CD28 antibodies were conjugated on the surface of the microparticles at predefined densities for T cell activation and signaling. Quantitative flow cytometry was used to estimate conjugation density of antibodies on the surface of microparticles (Table 1). These aAPCs actively engage with T cells (FIG. 36B).

TABLE 1
Conjugation Density of Antibodies
on the Surface of Microparticles

| | | Surface | Protein | |
|---|---|---|---|---|
| Signal | Diameter | Area | Density | Number of |
| Intensity | (μm) | (μm²) | (#/μm³) | Particles/test* |
| Low | 5.4 | 92 | 52 | 1,500,000 |
| Med | 5.4 | 92 | 457 | 1,500,000 |
| Hi | 5.4 | 92 | 3,515 | 1,500,000 |
| Med | 0.4 | 0.5 | 503 | 273,375,000 |
| Med | 16.5 | 855 | 480 | 160,661 |
| Med | 52.7 | 8,721 | 427 | 15,749 |
| Med | 247.6 | 192,500 | 465 | 713 |

*per 1.5 × 106 T cell. One and a half millions of 5.4 μm particles were used for T cell activation experiments with 1:1 ratio. This replicates the Dynabead for T cell activation protocol. The number of smaller or larger microparticles was normalized based on their surface area to deliver a similar level of stimulation.

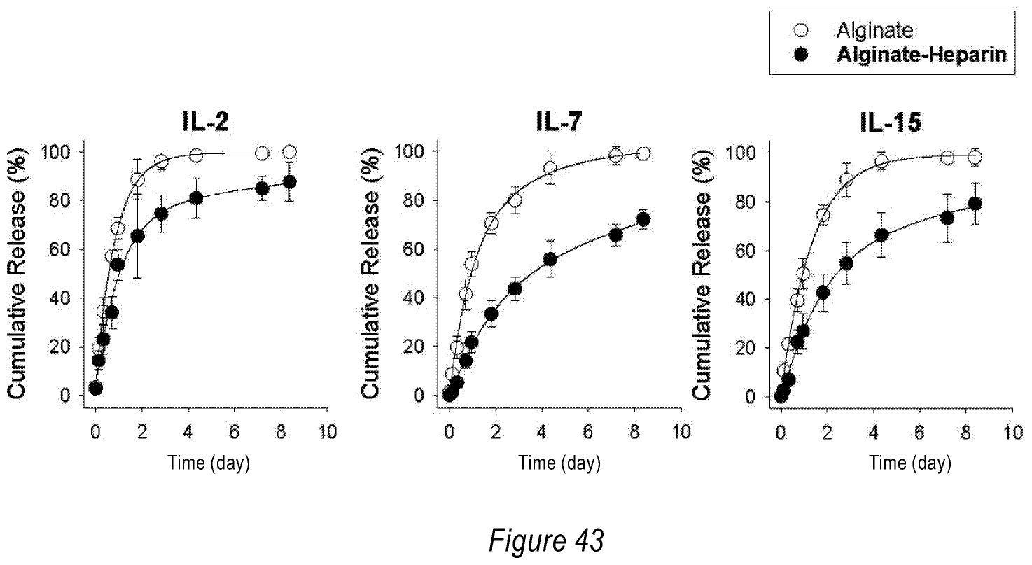

Sustained and prolonged exposure of T cells to cytokines allows tuning of immune responses. Recently it was showed that sustained release of cytokines from polymeric microparticles can regulate T cell fate, and the coating of microparticles with heparin increases the affinity of positively charged proteins or those with heparin-binding domains, prolonging their release. Here, heparin-functionalized alginate microparticles were synthesized and optimized to encapsulate cytokines IL-2, IL-7, and IL-15 (FIG. 43). IL-2 is well known to support the activation and differentiation of T cells. The related cytokines IL-7 and IL-15 not only instruct the generation of TMSCs from naïve precursors but also can enhance the self-renewal and expansion of TMSCs. Approximately 90% of the IL-2, IL-7, IL-17, and an antigen representative (Spike peptides from SARS-Cov-2) were released from the microparticles before day 7 (FIG. 36C).

To promote the development of TMSC, the activity of 4,6-disubstituted pyrrolopyrimidine was tested, i.e., TWS119, a potent inhibitor of the serine-threonine kinase glycogen synthase kinase-3β (GSK-3β) that induces Wnt-β-catenin signaling to enhance TMSC formation. This hydrophobic small molecule inhibitor was incorporated into poly(lactic-co-glycolic acid) (PLGA) nanoparticles by using a well-established nanoprecipitation method. To load these nanoparticles into the microparticles, monodispersed, TWS119-loaded and 150-nm (in diameter) PLGA nanoparticles were mixed with alginate-heparin prior to microparticle formation. The kinetics of TWS119 release from microparticles was studied in vitro (FIG. 36C), and showed that approximately 50% was released by day 2 and 80% by day 7 respectively.

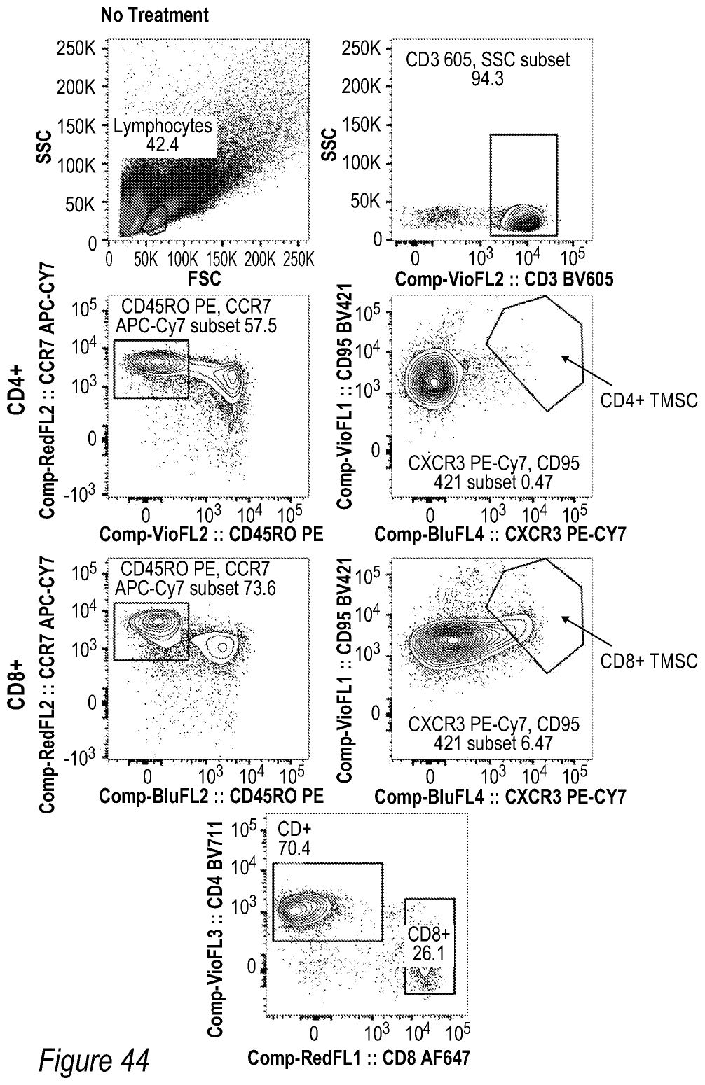

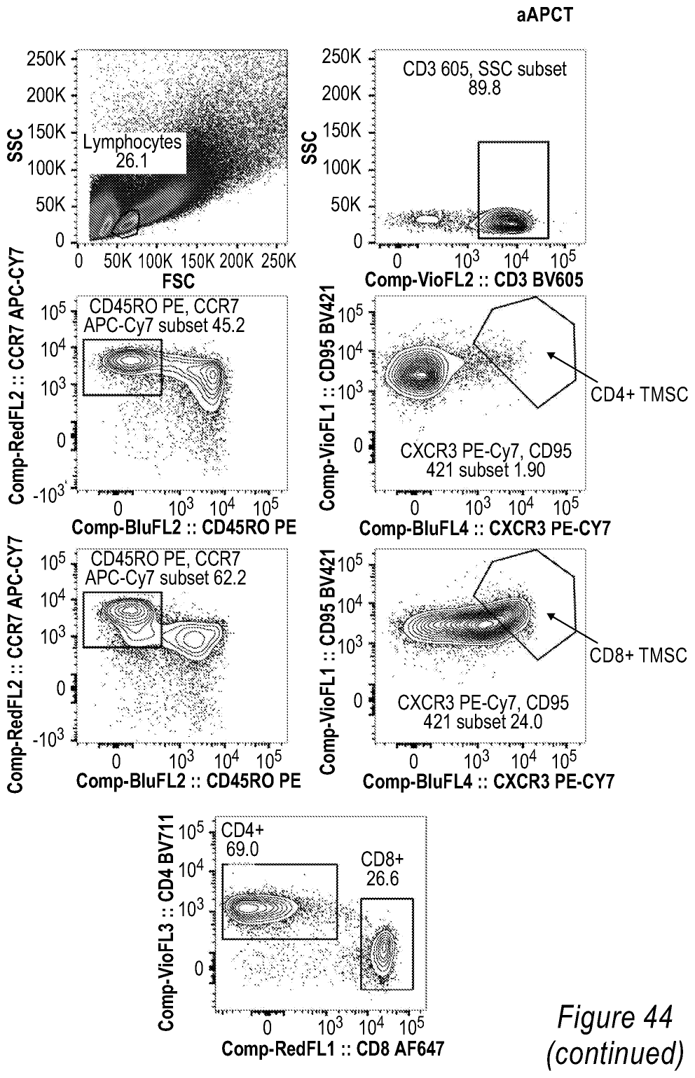

In previous work, it was found that raising or lowering the amount of T-cell receptor (TCR) signaling can substantially alter the differentiation of helper T cells. aAPCs with low, medium, and high coating densities of anti-CD3/anti-CD28 were developed herein. These aAPCs were formulated with TWS119-nanoparticles, Spike peptides, and IL-2, IL-7, and IL-15 cytokines. These aAPCs were co-cultured with peripheral blood mononuclear cells (PBMCs) from healthy human donors for four days and the phenotype of the resulting T cells was examined. Although the formation of both CD4⁺ and CD8⁺TMSC cells have been reported, the present example focused on CD8⁺TMSCs due to their direct antiviral capabilities. Human TMSC was defined here as CD8⁺ T cells that were CD45RO negative, CXCR3 positive, CCR7 positive, and CD95 positive (FIG. 44). Surprisingly, it was found that the formation of human TMSC was favored in conditions where antigen receptor signaling was low (FIG. 36D). It was also noted that the presence of TWS119 alone could not induce TMSC formation in vitro when T cells were stimulated by Dynabeads or by aAPCs that offered high TCR stimulation. Furthermore, IL-7 and IL-15 significantly expanded TMSCs in culture, but provided only a moderate advantage for the formation of TMSCs (FIG. 36E, FIG. 24). These data show that aAPCs with low-to-modest TCR stimulation and release of TWS119 and IL-2 can drive significant TMSC formation in vitro.

It was previously showed that T cells engaged aAPCs of different sizes and mechanical stiffnesses, resulting in an augmentation of TCR signaling. The induction of CD8⁺ TMSCs was then tested using aAPCs of various diameters in the range of sizes of immune cells (5-15 μm) and larger (250 μm). It was found that 5 μm-diameter aAPCs provided the maximal induction of TMSCs (FIG. 36F). A range of biologically relevant stiffnesses (elastic moduli) was also mimicked for the present aAPCs, based on mechanical stiffness of tissues T cells may experience in secondary lymphoid organs and peripheral tissues (10-200 kPa). It was found that TMSC formation is stiffness-dependent and was maximized by using aAPCs with the stiffness around 20 kPa (FIG. 36G). These data show that CD8⁺ T cells respond to the size and mechanical stiffness of aAPCs that modulate their differentiation potential into TMSCs, similar to the activation of CD4⁺ T cells. On the other hand, mechanically rigid, polystyrene aAPCs (Dynabeads) are particularly inefficient for TMSC formation. Based on these in vitro studies, 5-μm microparticles (20 kPa) with medium level of CD3/CD28 antibodies were used for the follow-up studies.

TMSC formation was further verified by examining the gene expression of transcription factors LEF1 and TCF1. Consistent with the flow cytometry analysis of TMSC surface markers (FIG. 36D), aAPCs with a sustained release of TWS119 significantly upregulated the gene expression of Lef1 and Tcf7 (encoding TCF1), which was much more effective than Dynabeads (FIG. 36H, FIG. 8).

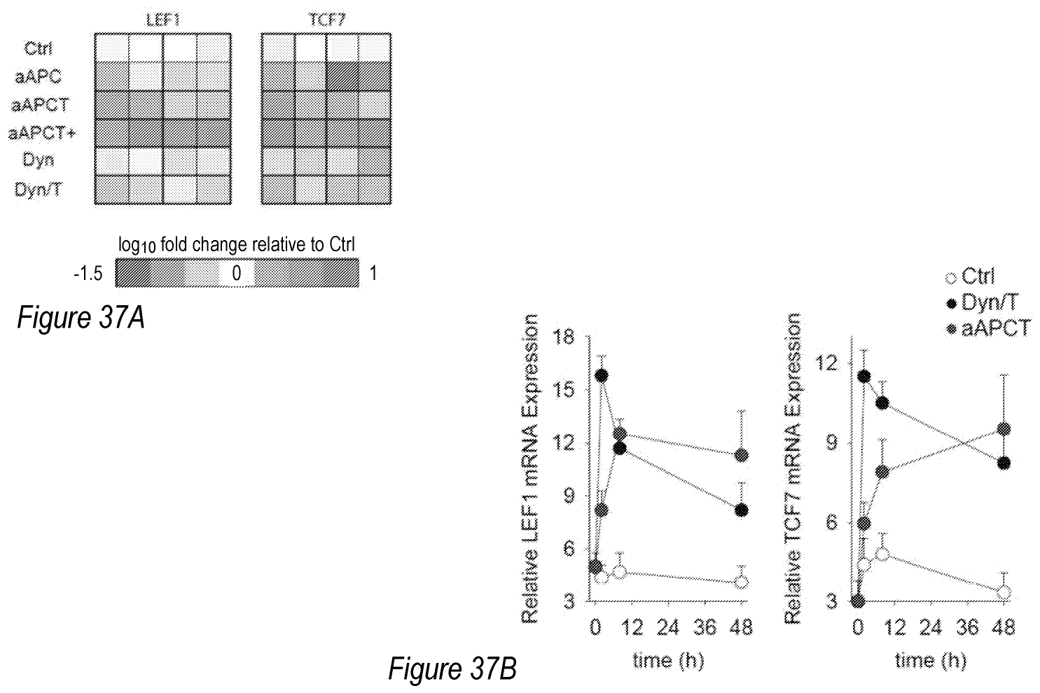

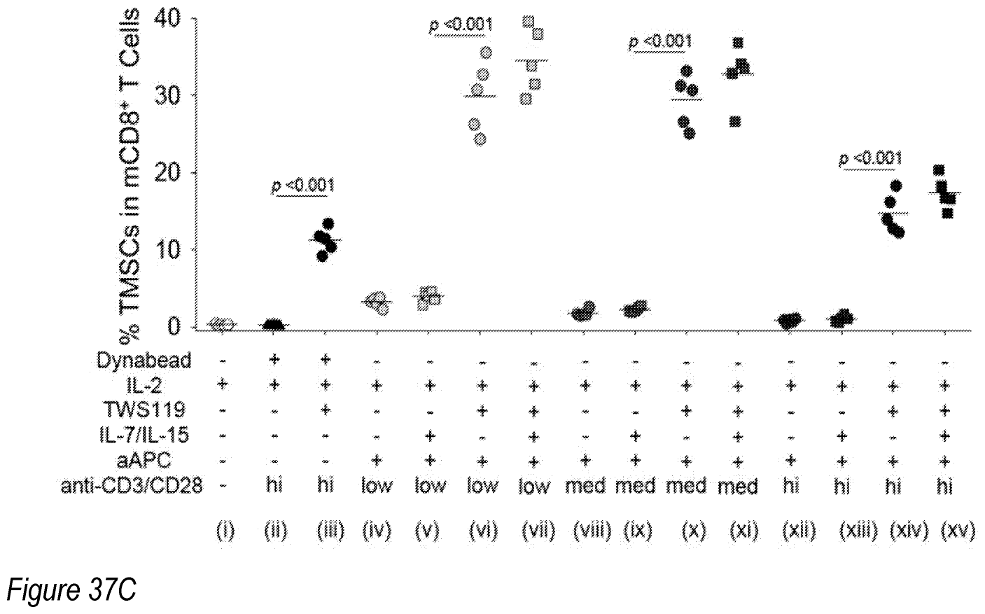

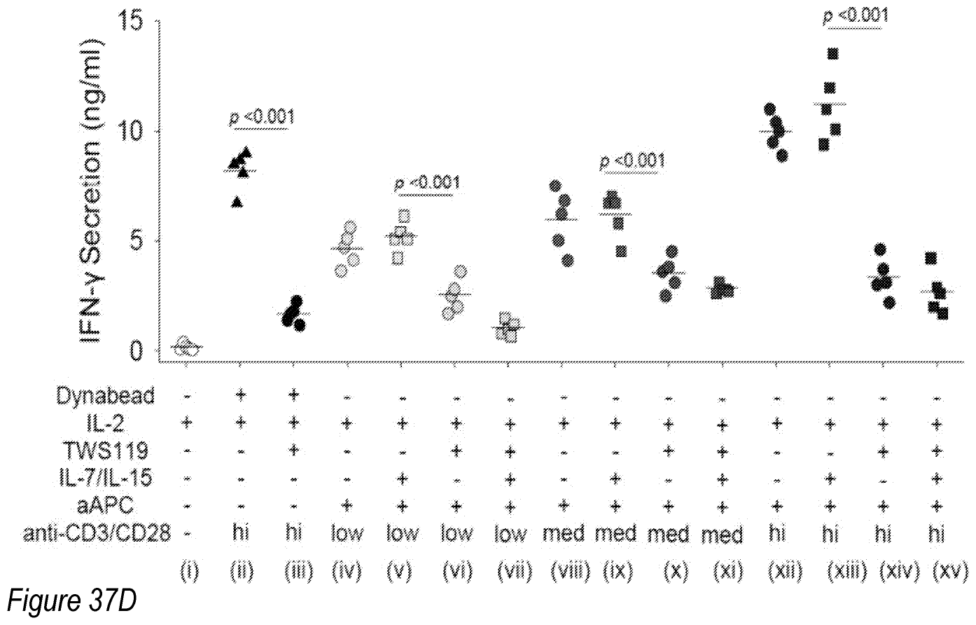

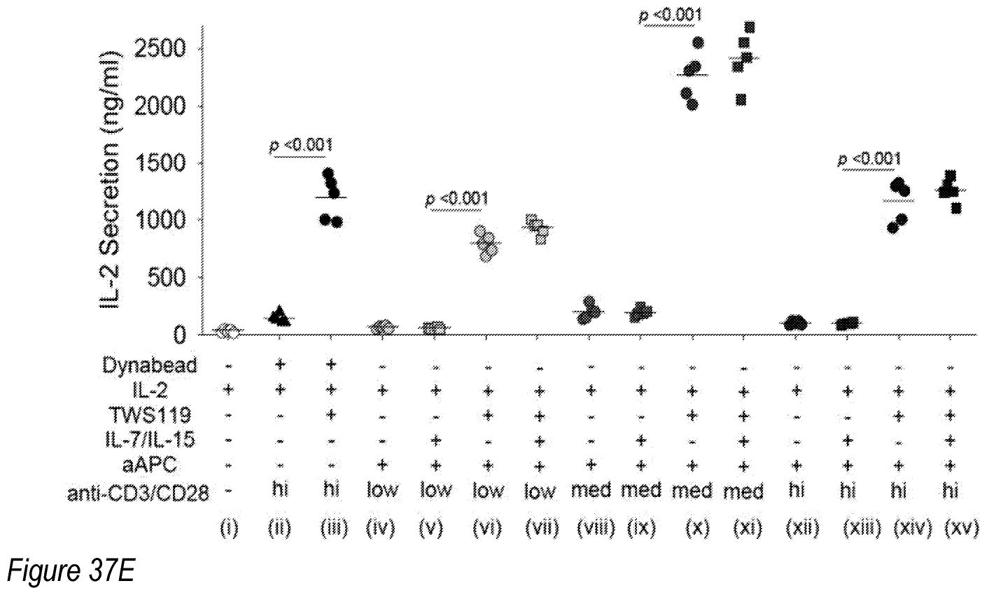

Murine version of aAPCs Induces a Robust Mouse TMSC Formation in vitro. A murine version of aAPC was also developed to assess response to vaccination in mice. TMSC in mice was identified as CD8⁺ T cells with CD44^{low} and CD62L^{high} that also expressed high levels of stem cell antigen-1 (Sca-1) (FIGS. 10, 17). Similar to that in human T cells, aAPCs with a sustained release of TWS119 induced the gene expression of Lef1 and Tcf7 after 48 hours (FIG. 37A), which was sustained after 48 hours (FIG. 37B, FIG. 9), while Dynabeads and soluble TWS119 induced a transient increase of Lef1 and Tcf7, which decreased rapidly after 12 hours.

In murine CD8⁺ T cells, low-to-moderate TCR stimulation maximized TMSC induction in the presence of TWS119, which was more robust than that in human CD8⁺ T cells (FIG. 37C). Previous findings show that IFN-γ^{low} and IL-2^{high} predicts a developmental fate towards memory, whereas IFN-γ production alone indicates differentiation towards Tc1 effectors (FIG. 14). TCR activation signals induced high levels of IFN-γ, but TWS119 inhibited IFN-γ expression and T cell proliferation (FIG. 37D, FIG. 11). Furthermore, medium/low level of TCR activation was most effective to increase IL-2 production in the presence of TWS119 (FIG. 37E). These results suggest that TWS119 suppresses the further differentiation of TMSC into effector cells.

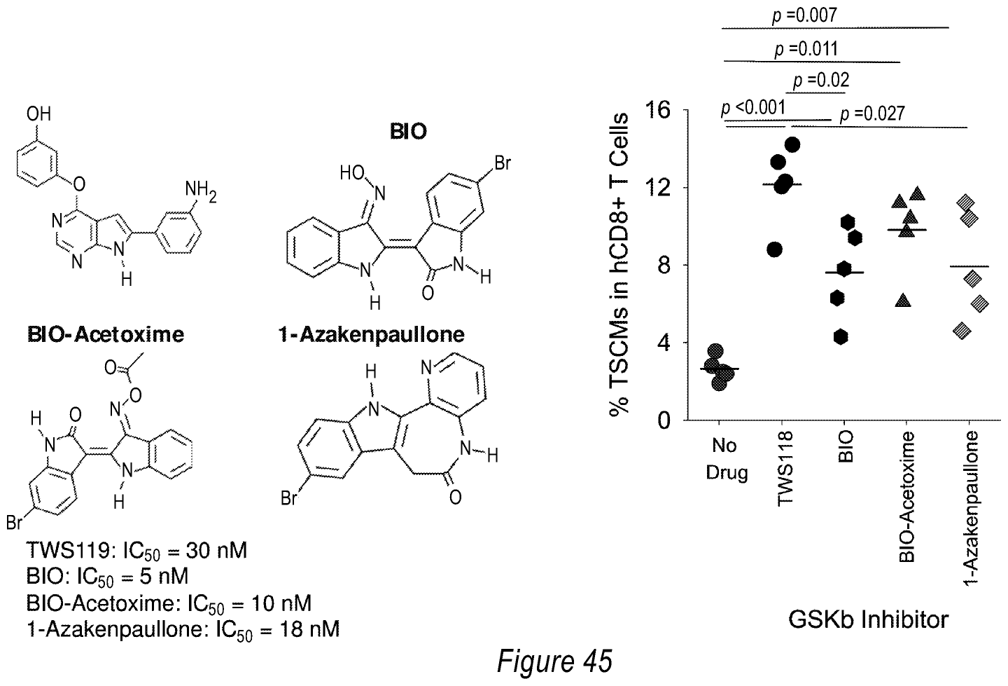

Other small molecules that inhibit GSK-3β were also tested, including: 6-bromo-substituted indirubin (BIO), BIO-acetoxime, and 1-Azakenpaullone. These small molecules were individually loaded into the present aAPC platform by encapsulating them first into PLGA nanoparticles, as it was done with TWS119. These aAPCs were then evaluated for the enhancement of TMSC formation in vitro. It was found that they also promoted the development of TMSCs (FIG. 45). TWS119 was used in subsequent studies due to its well-understood mechanism of action and its active development for humans as a cancer therapeutic (NCT01087294). These results confirm that, like in humans, low-to-moderate TCR stimulation in the presence of sustained inhibition of GSK-3β promotes the development of TMSC from mouse T cells.

Engineering aAPC Platform to Induce Antigen-Specific TMSC in vivo And Boost Vaccination Immunity. The ability to deliver vaccine antigens was then added to the present biomaterial platform. The well-established peptide pool of SARS-CoV-2 Spike glycoprotein was mixed with alginate-heparin conjugate prior to formation of aAPCs. Sustained release of these Spike peptides from the aAPCs was confirmed (FIG. 36B). To move towards in vivo translation, instead of activating purified T cells, the Spike peptide-eluting aAPCs were co-cultured with mouse splenocytes and resulted in robust TMSC formation (FIG. 19). The inclusion of Spike peptides also upregulated CD122 (IL-2/IL-15 receptor β chain) (FIG. 19), an important marker of antigen-specific TMSC formation.

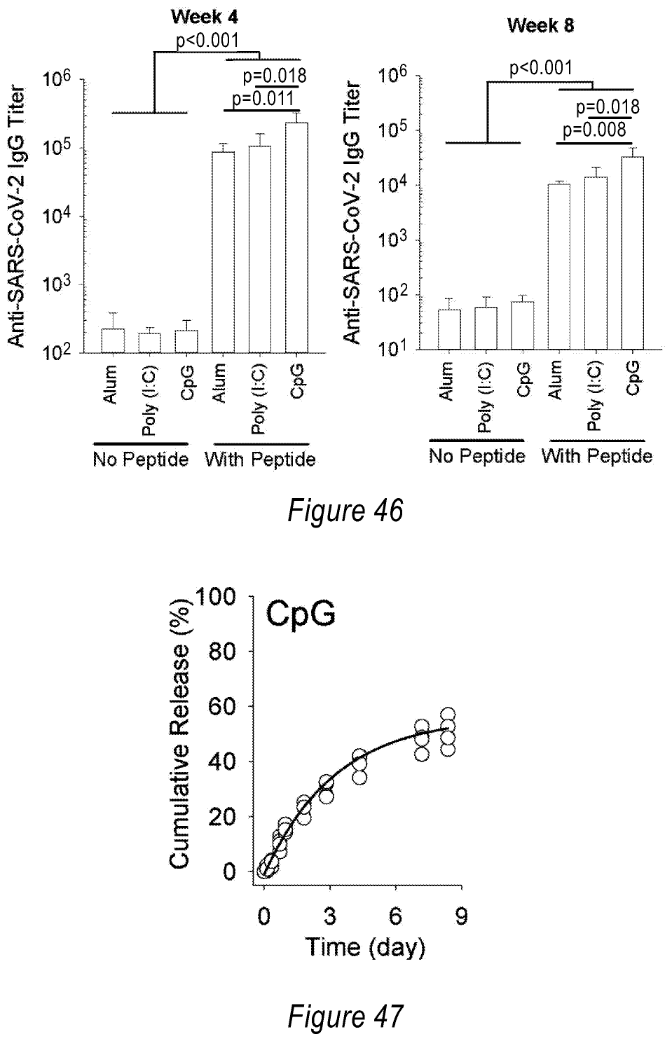

The efficacy of the present vaccine microparticles was then tested using a murine model, as previously reported for SARS-CoV and SARS-CoV-2. Early proof-of-concept animal data suggested the need for an adjuvant. The incorporation of three adjuvants into the formulation was individually tested: aluminum hydroxide (Alum), TLR agonists poly(I:C) and CpG. While all three adjuvants induced strong humoral response, CpG generated stronger immunity than poly(I:C) and Alum (FIG. 46). Microparticles enabled a sustained release of CpG (FIG. 47), and CpG was chosen for subsequent work.

Since IL-7/IL-15 only offered moderate improvement of TMSC induction in vitro, the present aAPC was simplified by excluding IL-7/IL-15 for in vivo studies. Considering the potential presence of T cell activation signals in vivo, the CD3/CD28 antibodies were also eliminated from the formulation in one experimental group, i.e., Vax-T. A single injection of aAPC (alginate-heparin microparticles (5 μm, 20 kPa) was compared with medium level of anti-CD3/anti-CD28 surface antibodies, releasing SARS-CoV-2 peptides/IL-2/CpG), aAPCT (aAPC+TWS119), or Vax-T (aAPCT without CD3/CD28 antibodies) with bi-weekly injections of the soluble components (SARS-CoV-2 peptides+IL-2+CpG with and without TWS119), and investigated TMSC formation and humoral responses in the mouse model.

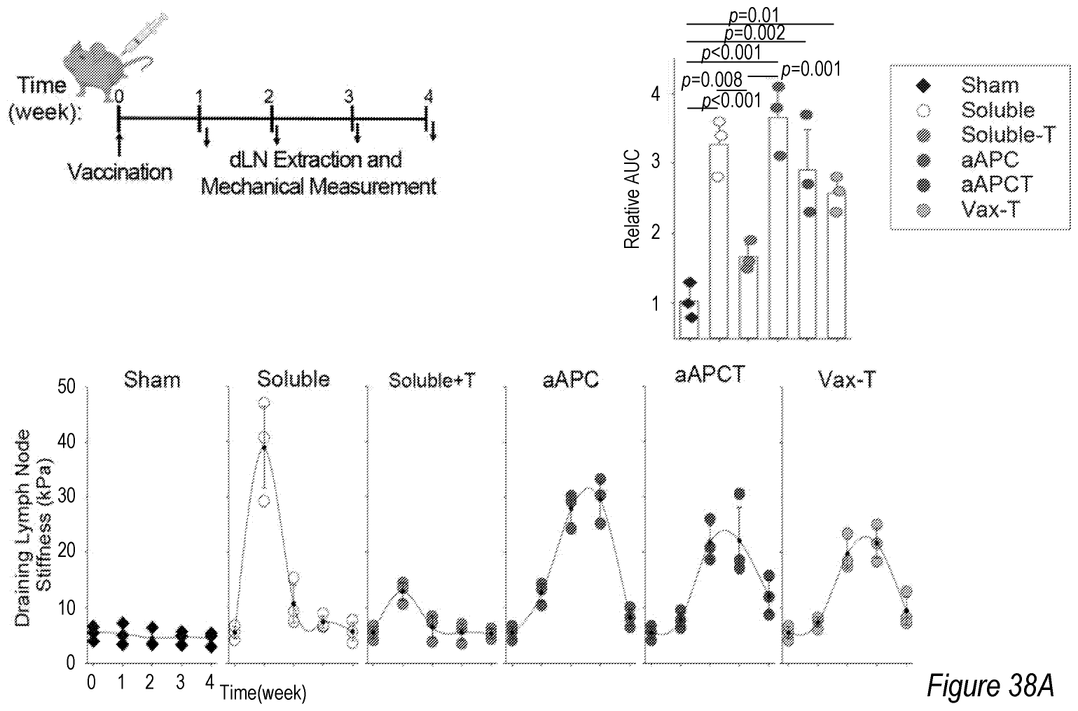

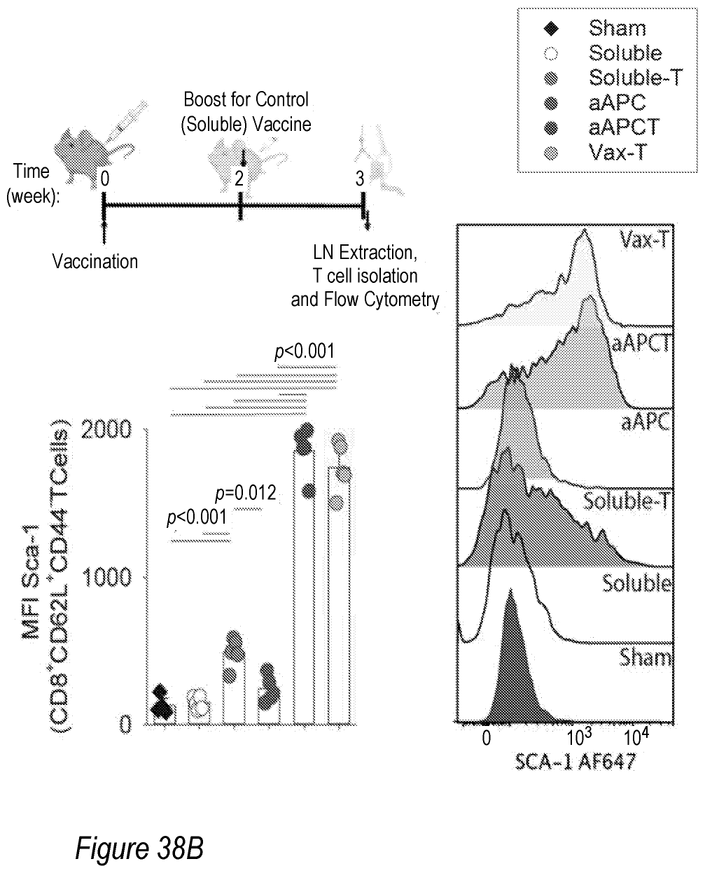

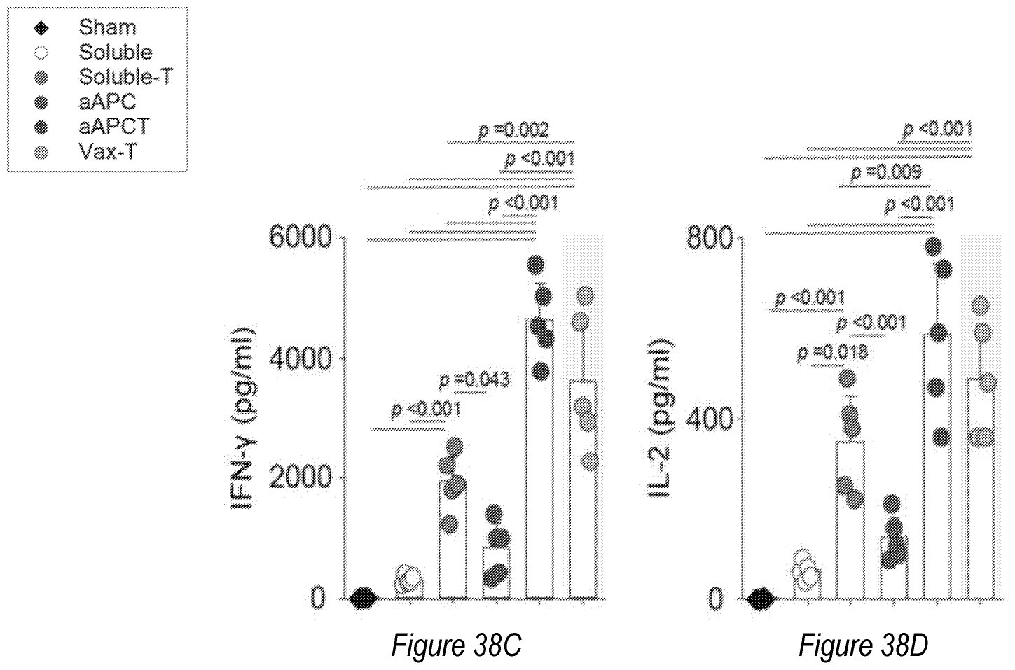

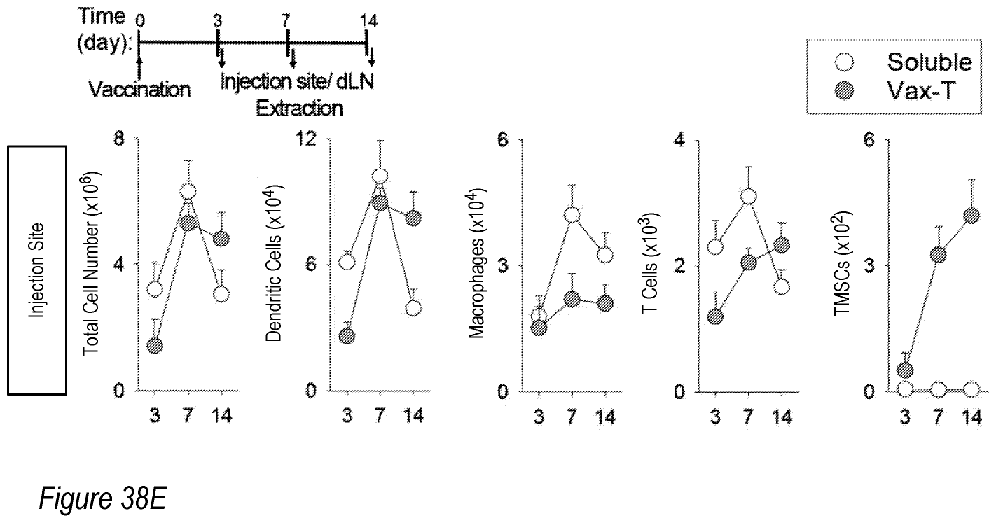

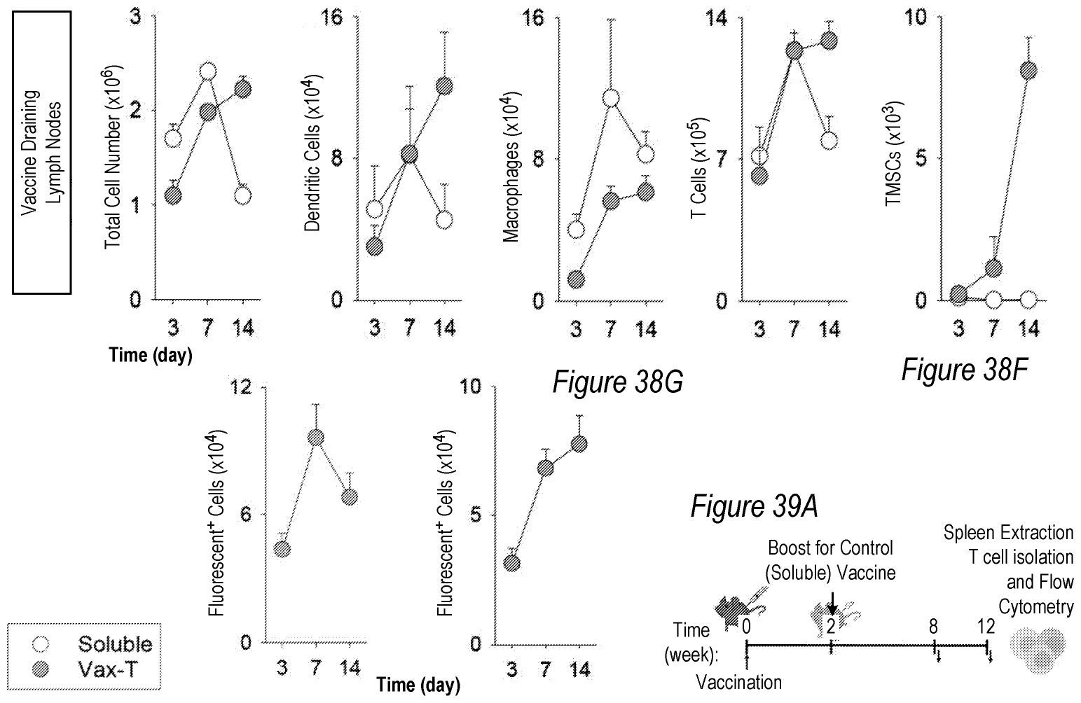

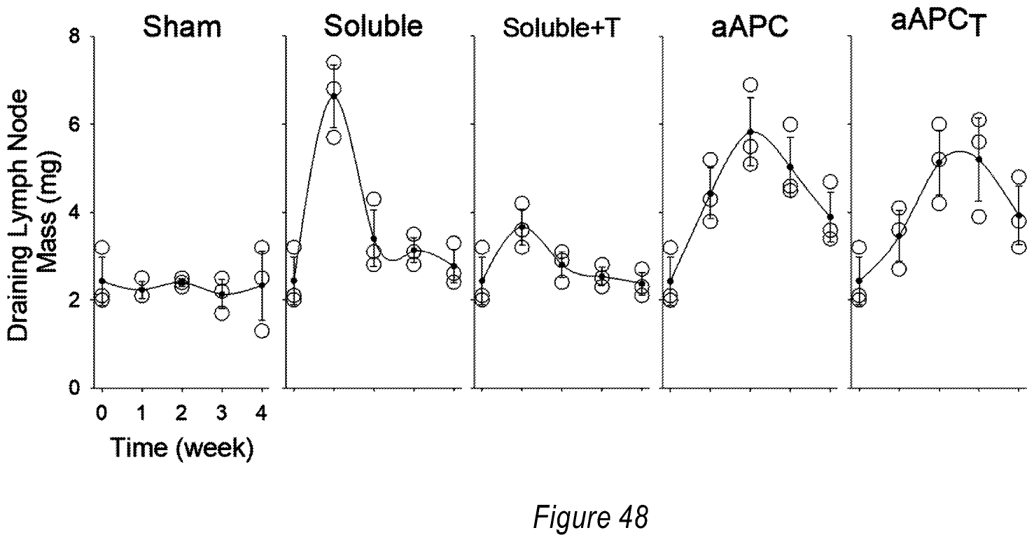

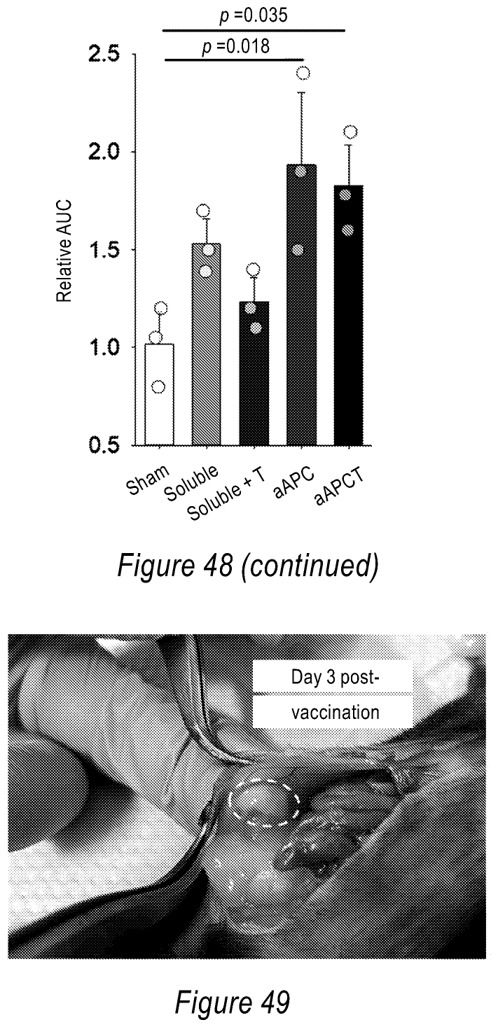

Upon vaccination, the change in stiffness of draining lymph node (dLN) was first examined at different time points. It has been known for centuries that infection and consequent inflammation elicit rigidity of regional LNs, and recent work revealed that this mechanical rigidity promoted T cell activation. Vaccines, including mRNA COVID vaccines, can also induce mechanical stiffening of lymph nodes. The physiological change in bulk stiffness of dLNs was measured over the course of four weeks (FIG. 38A). For soluble injections (CpG/IL-2/Spike peptide), an ˜8-fold increase in stiffness was detected one week after vaccination, followed by a rapid return to baseline (FIG. 38A, soluble). The addition of TWS119 significantly dampened the induction when administered in solution (FIG. 38A, soluble-T). Delivery of these ingredients within the microparticle platform sustained LN stiffness for more than three weeks (FIG. 38A, aAPC, aAPCT and Vax-T). The integrated stiffness over time was comparable to the soluble formulation. The mass of dLNs, which is driven by the proliferation of stromal cells and lymphocytes, showed similar trends after vaccination (FIG. 48). These results showed that a sustained release of vaccine components by aAPCs elicited a sustained activation of dLNs beyond that induced by conventional bi-weekly injection of soluble vaccines.

To analyze the development of SARS-CoV-2-specific T cells, mice were immunized with various vaccine formulations, and dLNs were extracted three weeks after immunization. Flow cytometry revealed in vivo induction of TMSCs under the conditions where TWS119 was delivered (FIG. 38B). Importantly, the development of TMSC was much higher for the microparticle platforms than for the soluble components, and Vax-T had similar effect as aAPCT, suggesting that in vivo delivery of CD3/CD28 antibodies was not necessary. To evaluate the ability of these TMSC to respond to spike antigen, putative TMSC (naï-like CD8⁺ T cells that are CD62L⁺CD44⁻) was sorted, restimulated with the Spike peptide pool overnight, and cytokine production was evaluated. It was found that TMSCs elicited by TWS-119 delivery co-produced both IFN-γ (FIG. 38C) and IL-2 (FIG. 38D), suggesting the fast recall capability of TMSC that makes them desirable vaccine responders.

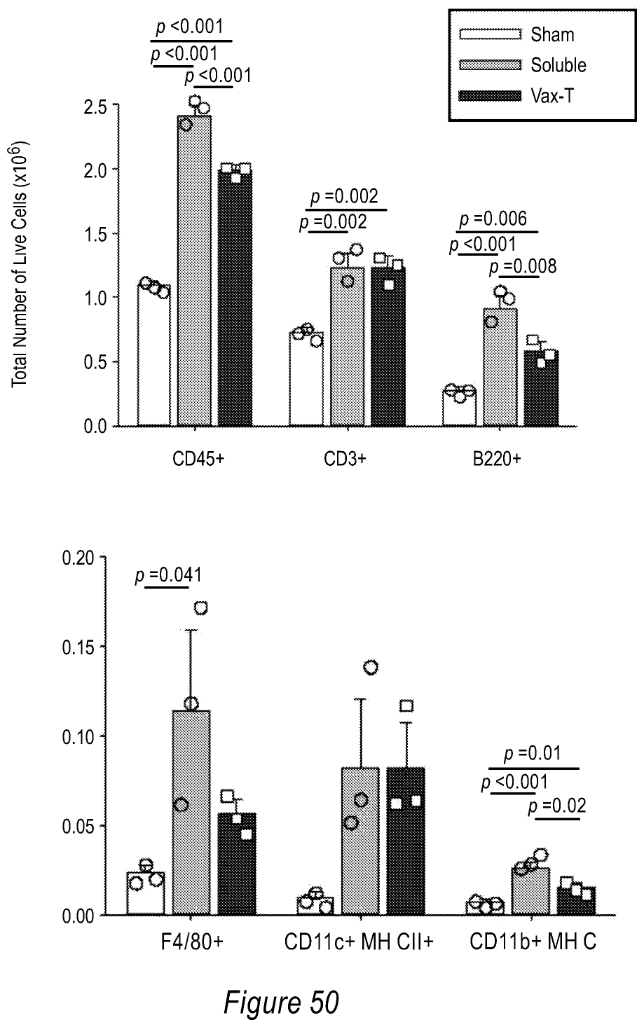

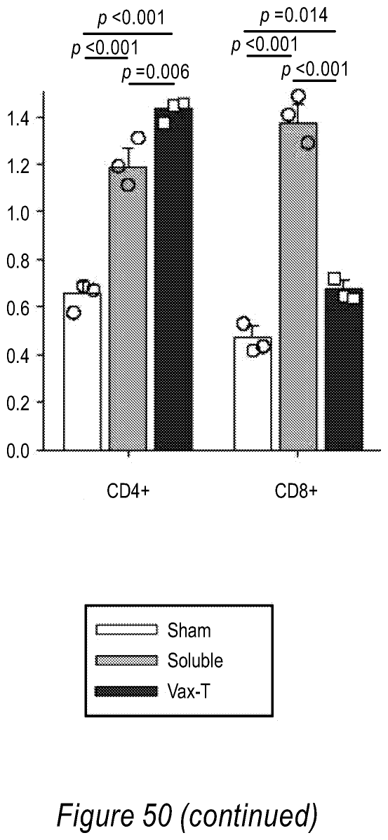

The cellular responses at the injection site and dLN to soluble and Vax-T vaccines as representatives were then compared. Examination of the retrieved samples from injection sites (FIG. 38E, FIG. 49) as well as vaccine-dLNs (FIG. 38F, FIG. 50) on day 3, 7, and 14 post-vaccination indicated that Vax-T was more effective than the bi-weekly injection of soluble Spike peptide/IL-2/CpG to increase a sustained recruitment of dendritic cells and T cells (but not macrophages) at day 14, and Vax-T induced a robust TMSC formation at both the injection site and in dLNs at day 7, which further increased at day 14. Dendritic cells and macrophages can digest the vaccine components locally and carry them to the dLNs. As shown in FIG. 38G, the number of cells carrying fluorescent PLGA nanoparticles increased between day 3 and 7 at both the injection site and dLN; interestingly, at day 14, the number of fluorescent cells decreased at the injection site but increased in dLN, suggesting cell trafficking from the injection site to dLN. These results suggest that both migrated cells and the transported TWS119 nanoparticles into dLNs could have contributed to the further induction of TMSCs.

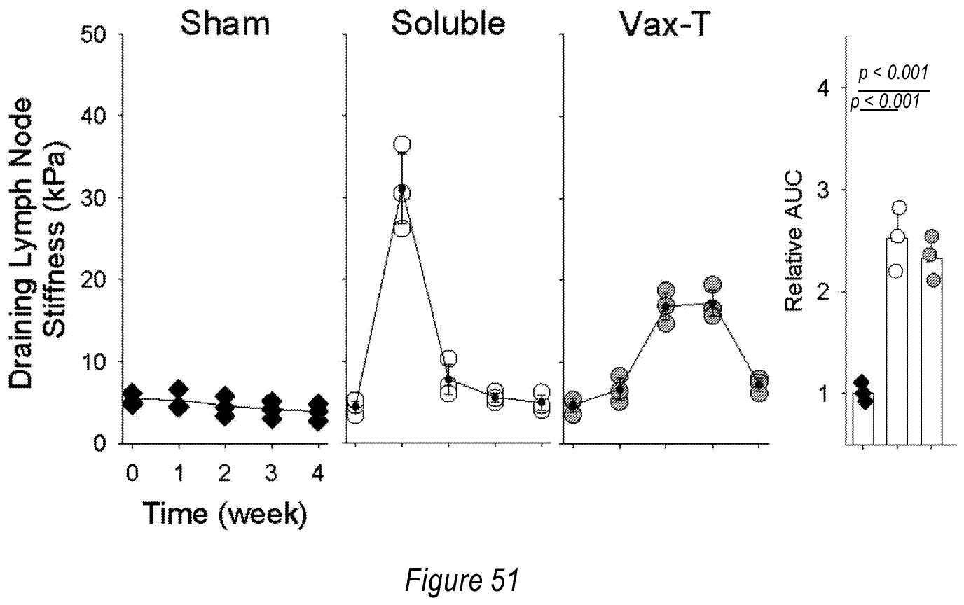

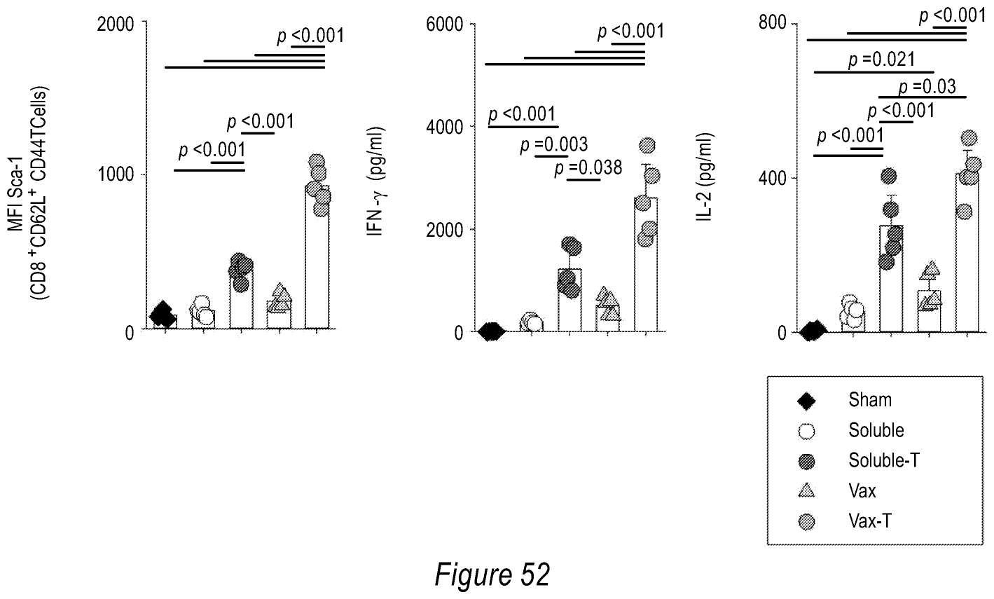

Biomaterial-Based Vaccines Can Enhance Antigen-Specific T Cell Long-Term Responses in Young and Aged Mice. Because vaccine response is far weaker in the elderly compared to the younger populations, the ability of the present microparticle vaccine platforms to elicit TMSC in elderly mice was next ascertained, comparing wildtype C57BL/6 young (6-10 weeks-old) and aged (10-13-month-old) mice. Within 4 weeks post-vaccination, similar trend on dLN stiffening and TMSC induction (week 3) was demonstrated when vaccinating aged mice with Vax-T, in comparison with soluble vaccine controls (FIGS. 51,52), although the fold induction of TMSCs was about half of that in young mice (FIG. 38B).

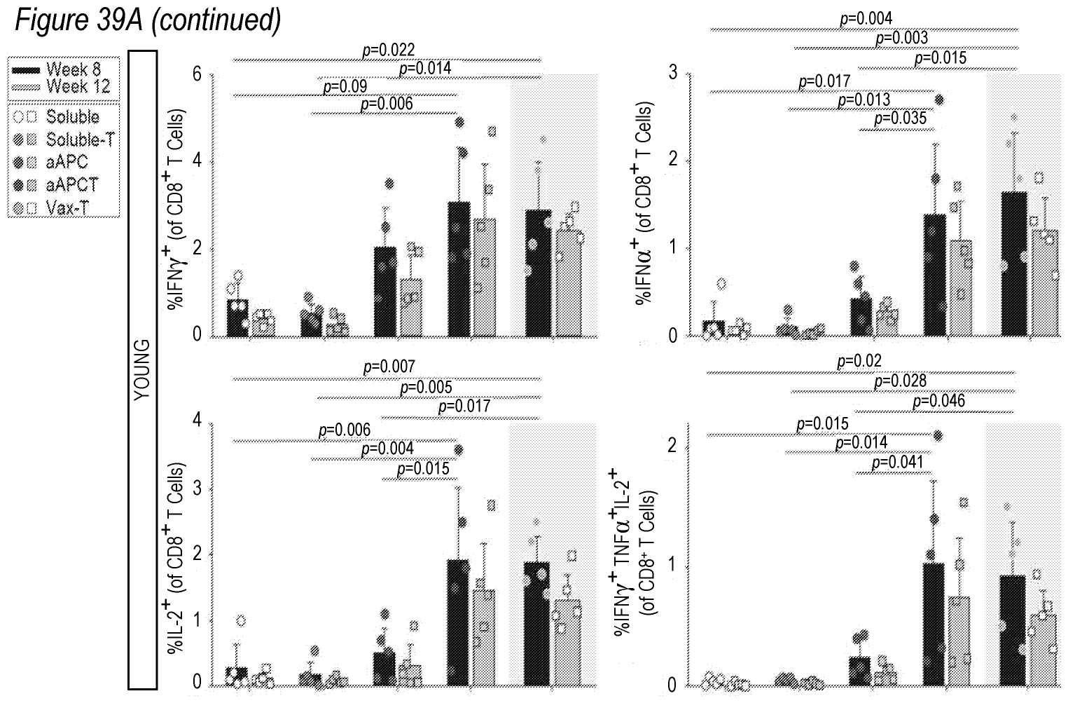

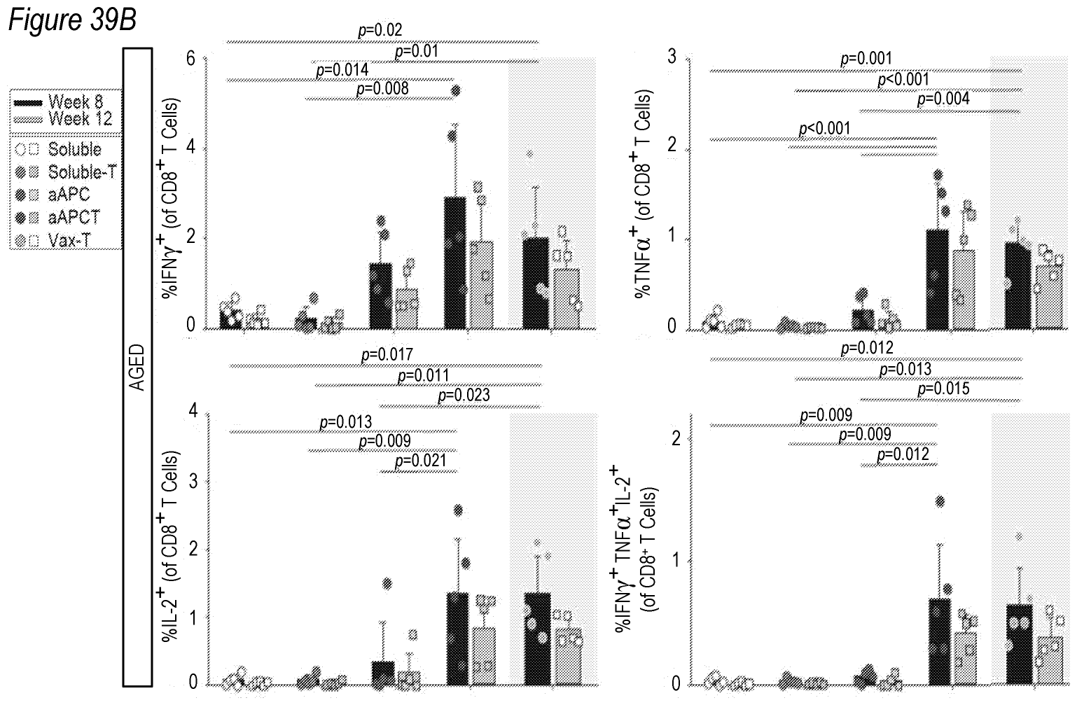

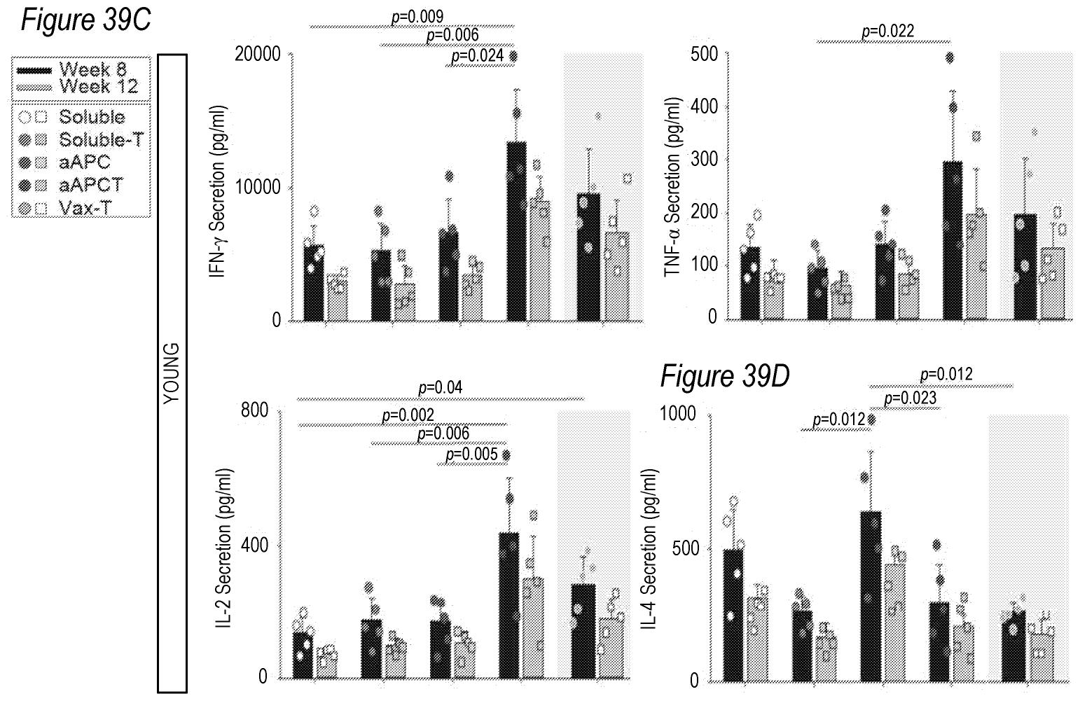

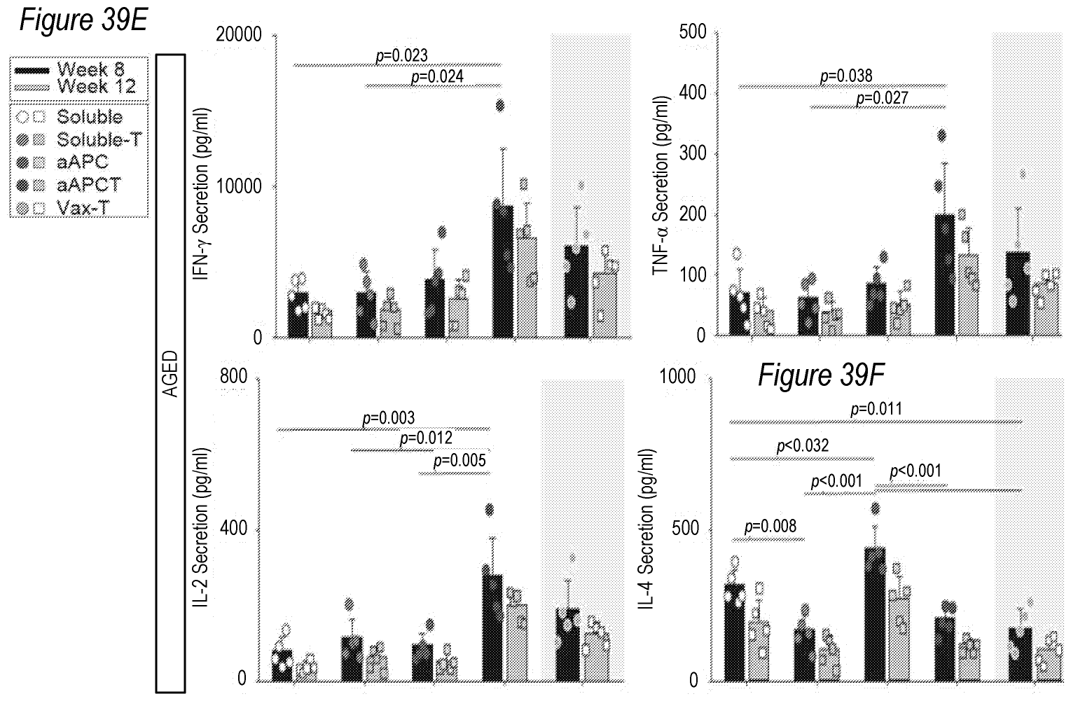

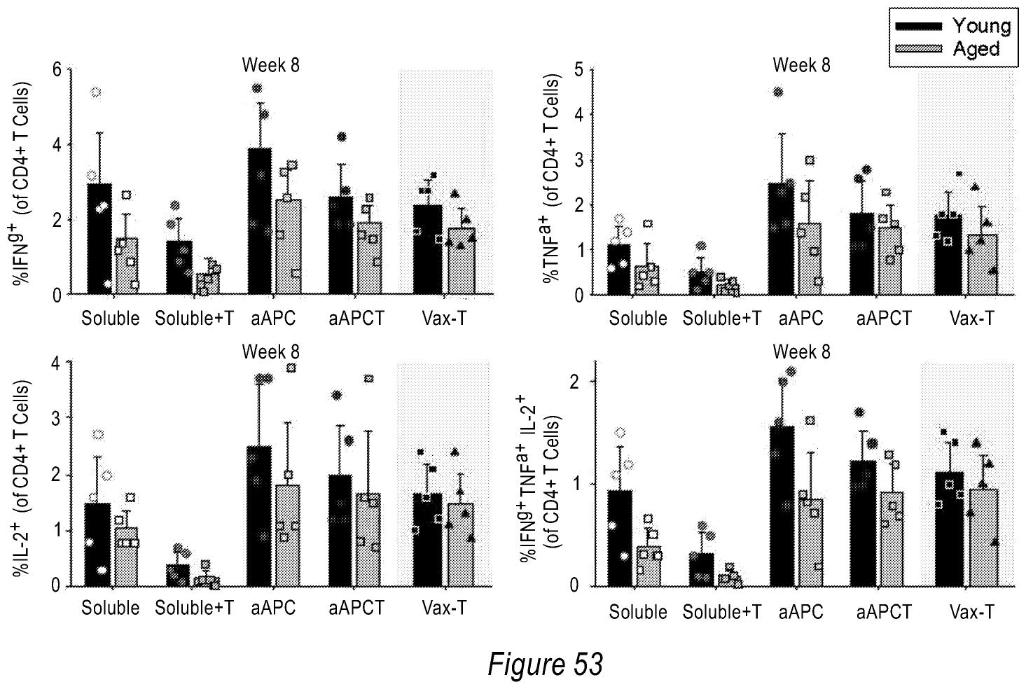

Since T cell responses to vaccines are more sustained than elevation of antibody titers and play an important role in clearing virally infected cells, the long-term responses of T cells after 8 weeks in young and aged mice were examined. These mice were vaccinated twice with soluble vaccine components (Spike peptides, cytokines, adjuvant, and ±TWS119) (week 0 and week 2) as compared to a single immunization of microparticle-based vaccines (aAPC, aAPCT, and Vax-T) (FIG. 39A). To determine whether the present vaccine platform could induce Spike peptide-specific T cell responses, mice were immunized as described above, and after eight and twelve weeks, T cells were purified from the lymphoid tissues, and cytokine production was evaluated by overnight stimulation with SARS-CoV-2 Spike peptides. The population of cytokine-producing (IFN-γ, TNF, and IL-2) cells among responding CD8⁺ T cells was measured. While vaccination with TWS119 induced polyfunctional T cells (producing IFN-γ, TNF, and IL-2) on week 8 and 12 after immunization in young mice (FIG. 39A), the sustained release of TWS119 (by aAPCT and Vax-T) greatly outperformed two injections of the soluble vaccines. Similar trend was observed in aged mice with slightly lower T cell responses (FIG. 39B). On the other hand, for CD4+ T cells, polyfunctional recall responses improved upon sustained delivery by aAPCs but did not apparently require TWS119 (FIG. 53). This result shows that in vivo development of CD8⁺ TMSC by aAPCs results in robust recall responses after a single immunization.

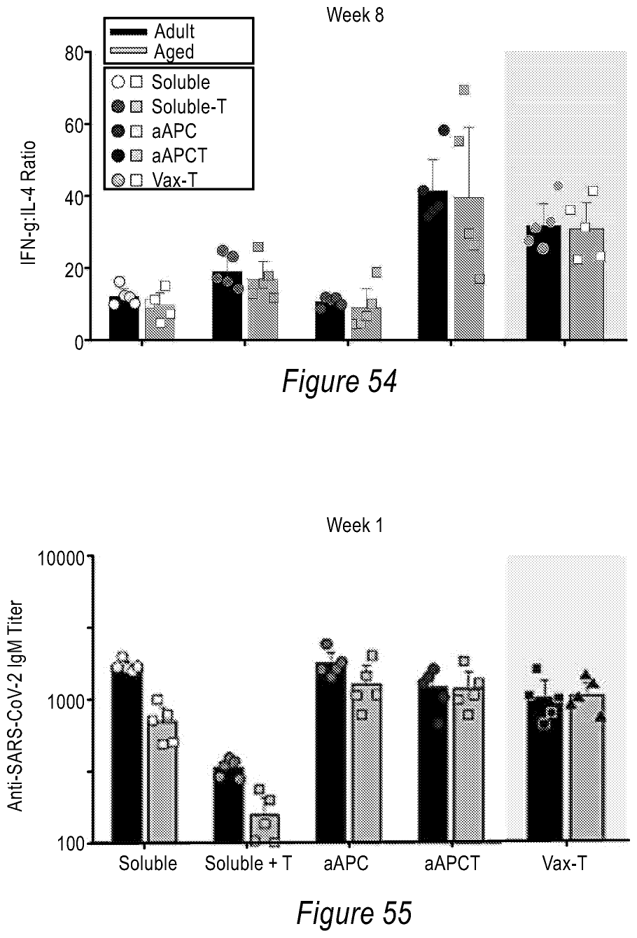

To further investigate the nature of cellular immunity elicited by the vaccines, mice were vaccinated as above, and after 8 and 12 weeks, the level of cytokines secreted by splenocytes was measured following three days of restimulation with the overlapping Spike peptide pool. Cytokine responses from splenocytes vaccinated with aAPCT showed significantly higher production of Th1 cytokines, specifically IFN-γ, TNF, and IL-2, compared to the other groups in young (FIG. 39C) and aged (FIG. 39E) mice. However, mice vaccinated with microparticle formulations lacking TWS119 secreted high levels of IL-4, the key Type-2 cytokine (FIGS. 39D, 39F). Regardless, the ratio of IFN-γ to IL-4 was significantly higher for aAPCT and Vax-T compared to the control soluble formulations (FIG. 54).

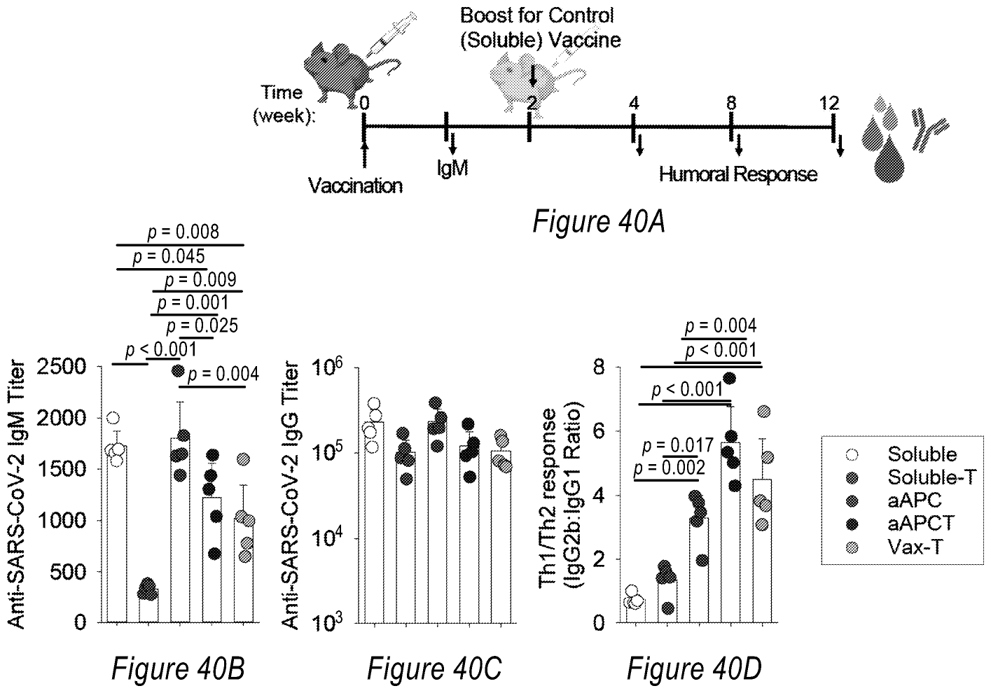

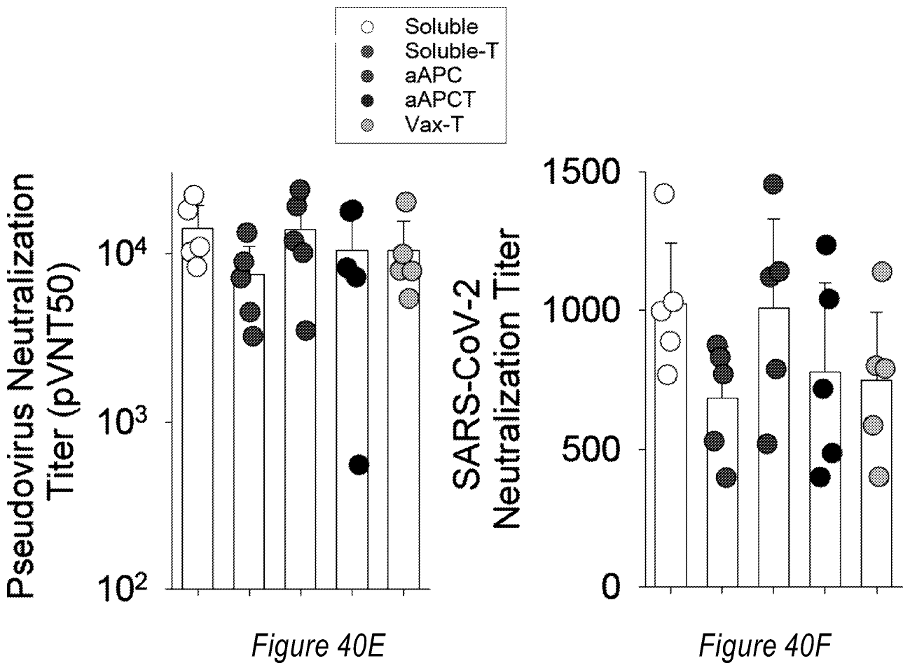

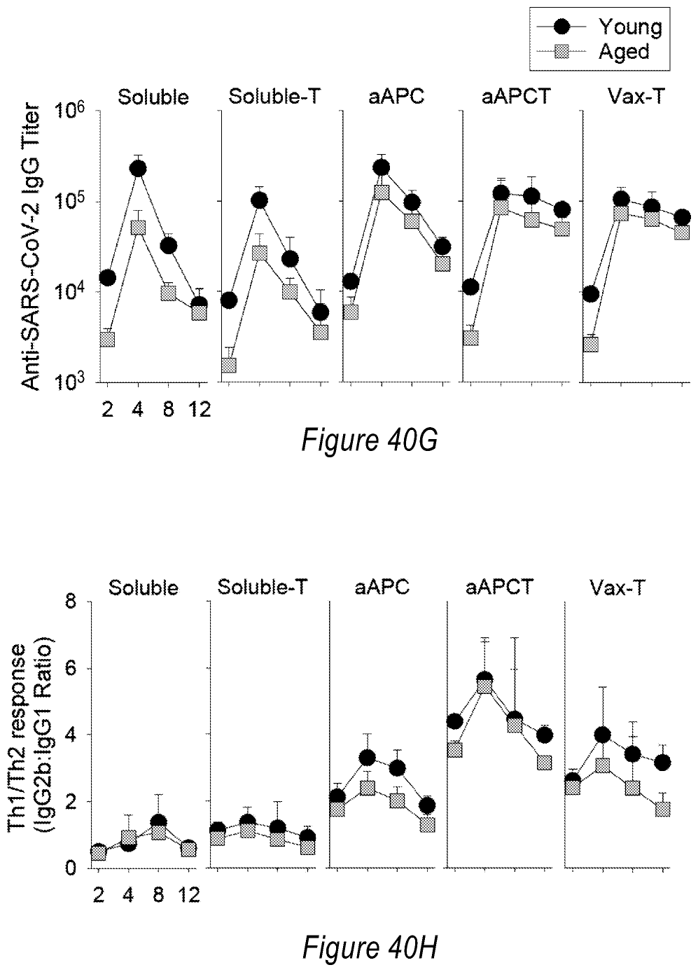

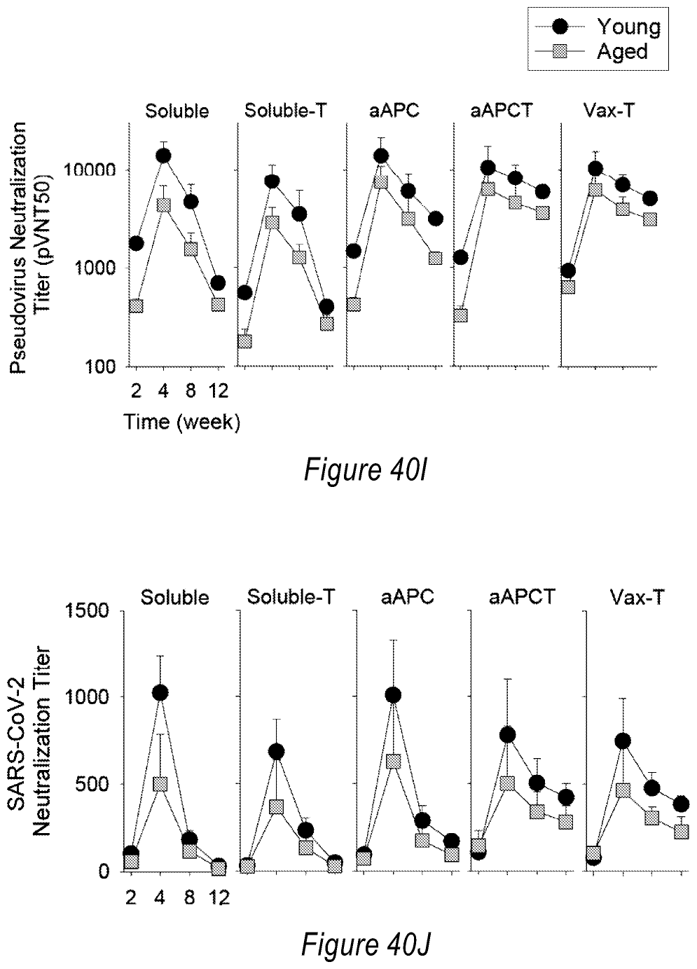

Biomaterial-Based Vaccines Can Sustain Humoral Responses in Young and Aged Mice. Looking at early humoral responses (FIG. 40A), it was found that all mice showed robust Spike protein-specific IgM on day 7 after vaccination, except for the control mice that received mock immunization (no spike peptides) or soluble formulations with TWS119; the reduction of IgM responses by TWS119 was not seen in the mice that received prolonged release (FIG. 40B, FIG. 55).

Mice that received microparticle vaccinations (aAPC, aAPCT, and Vax-T) and soluble formulations showed similar levels of SARS-CoV-2-specific IgG in their serum four weeks after vaccination (FIG. 40C). At week 12, the responses elicited by a single injection of aAPCT or Vax-T were much higher than other groups; in addition, aAPC vaccination lacking TWS119 had higher IgG titer than that elicited by two injections of the soluble vaccine components (FIG. 40G). Remarkably, elderly mice showed humoral responses nearly comparable to the younger mice at all time points when immunized with aAPC, aAPCT or Vax-T. In contrast, vaccination with the soluble components resulted in lower humoral response in the elderly mice (FIG. 40G).

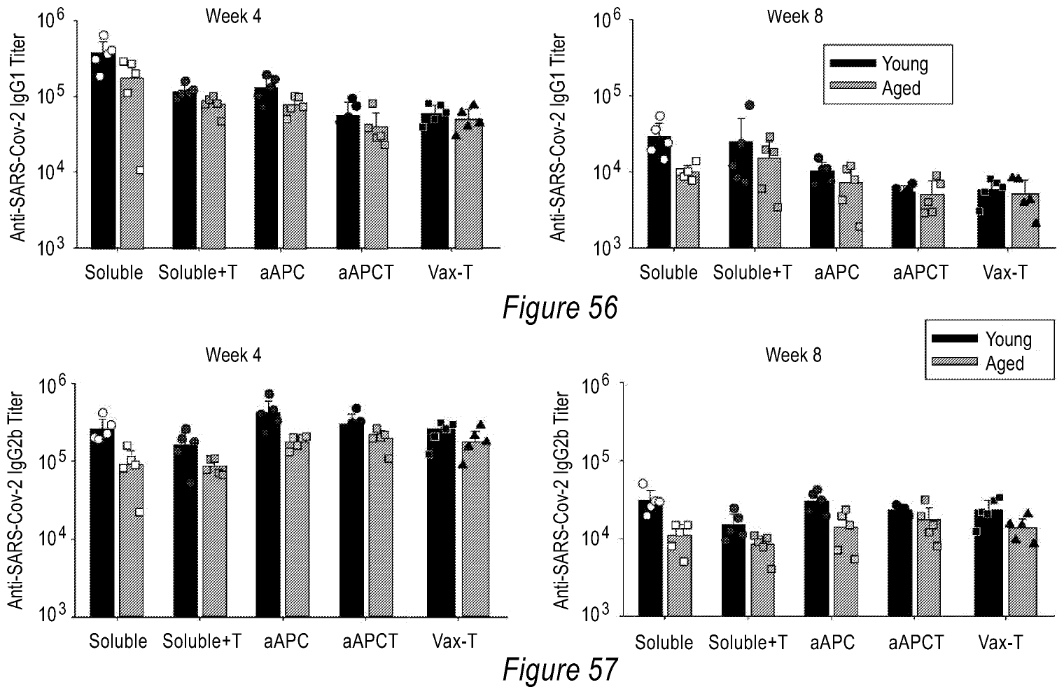

The production of different IgG subclasses by responding B cells, driven by cytokines produced by differentiated T follicular helper cells (Tfh1 versus Tfh2), have different degrees of protection for viral responses for as yet unclear reasons. One report showed that IgG2 subsets in mice (corresponding to Type-1 immunity) exhibited effective anti-viral activity as compared to Type-2 immune responses. To assess if there was any bias in the Spike-peptide-specific IgG responses, the spike-specific IgG subclasses were tested in the immunized mice above. It was found that the microparticle formulations (aAPC, aAPCT and Vax-T) showed significantly lower IgG1 titers (corresponding to Type-2 immunity) compared to the mice that were immunized with soluble formulations (FIG. 56). On the other hand, aAPC, aAPCT and Vax-T groups elicited stronger IgG2b subclass production (driven by Tfh1 responses) (FIG. 57). The ratio of IgG2b to IgG1 titer indicated a strong bias towards Type-1 immunity for aAPC, aAPCT and Vax-T immunized mice, while soluble formulations produced either a balanced Th1/Th2 profile or Type-2-dominated response at 4, 8, and 12 weeks after vaccination (FIGS. 40D, 40H). Elderly mice exhibited lower enhancement of Type-1 compared to younger mice (FIG. 40H). Remarkably, even at the 12-week time point, the elderly mice showed higher Type-1 enhancement from the microparticles bearing TWS119 than at any time point in the control groups (FIG. 40H). These results showed that the present microparticle platform preferentially elicited Type-1 subclasses of specific antibodies.

The ability of Spike peptide-specific antibodies to neutralize SARS-CoV-2 pseudovirus binding to HEK293 cells expressing angiotensin-converting enzyme 2 (ACE2) was next tested, as previously described. The results demonstrated that all the groups exhibited robust Spike peptide-specific neutralizing antibody responses at week 4 (FIG. 40E), except for the controls that received mock immunization without spike peptides (data not shown). However, it was found that, at week 12, the microparticle-based formulations that included prolonged release of TWS119 (aAPCT and Vax-T) provided a significantly more (p<0.001) protracted neutralization response as compared to soluble vaccinations or the aAPC microparticles that lacked TWS119 (FIG. 40I). The aged mice showed similar trend as in young mice but slightly lower responses for all conditions (FIG. 40I). These observations demonstrate why looking at long time points like week 12 are critical, as all vaccination strategies produced comparable neutralizing antibodies in the early timepoint (week 4). The effectiveness of the vaccines was also tested in vitro by using SARS-CoV-2 surrogate neutralization assay to examine the blockage of SARS-CoV-2 Spike RBD-HRP binding to ACE2 precoated plates. The neutralization assay in cell culture showed the same trend (FIGS. 40F, 40J) as pseudovirus neutralization experiments.

Taken together, these data demonstrate that vaccination approaches that elicit TMSC enabled strong and sustained production of Th1-biased antibodies against SARS-CoV-2 Spike protein. In contrast, mice that received two doses of soluble vaccine formulations could not sustain neutralizing IgG responses. There were no significant differences in the neutralizing activity of the soluble formulations compared to the aAPC/Vax-T vaccines at earlier timepoints. The difference was more significant at later timepoints (e.g., 12 weeks) in both young and aged mice, although the responses in aged mice were lower. Taking into account both humoral responses (increased IgG2b:IgG1 isotype ratio, FIG. 40H) and cellular responses (cytokine secretion after restimulation, FIGS. 39C, 39D), the present aAPCT and Vax-T elicited TMSC and enabled sustained and robust Th1-biased responses.

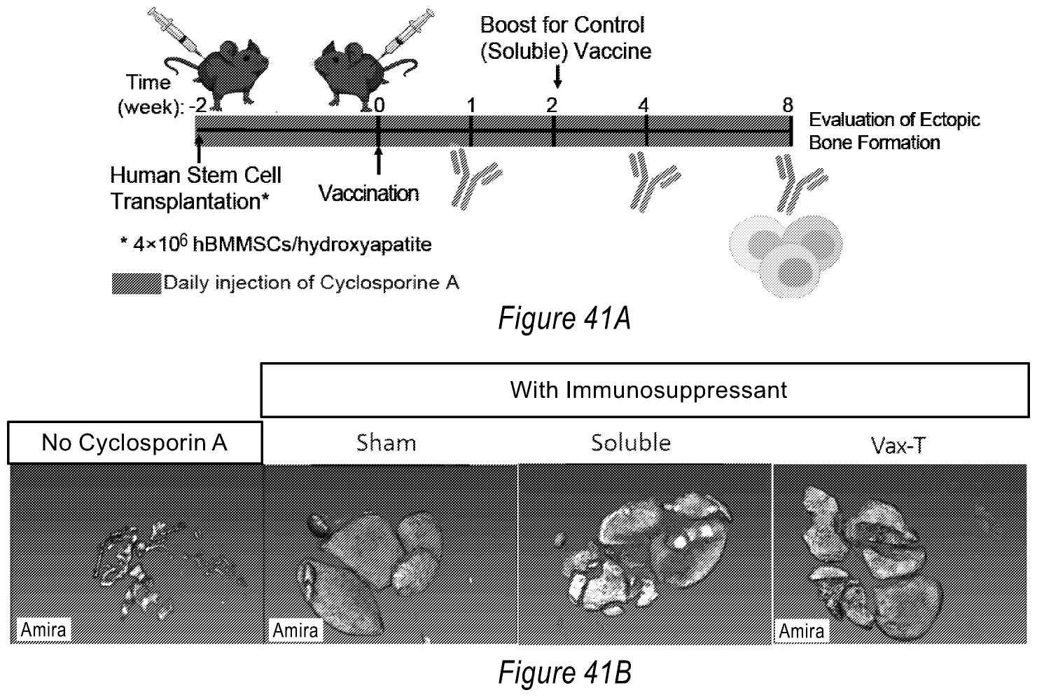

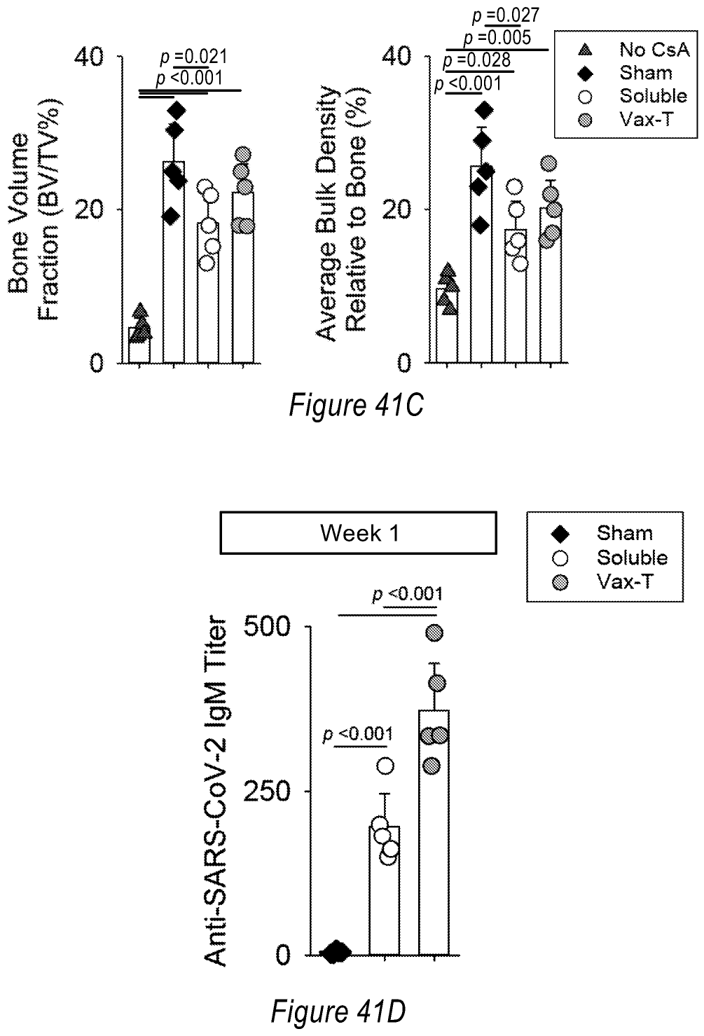

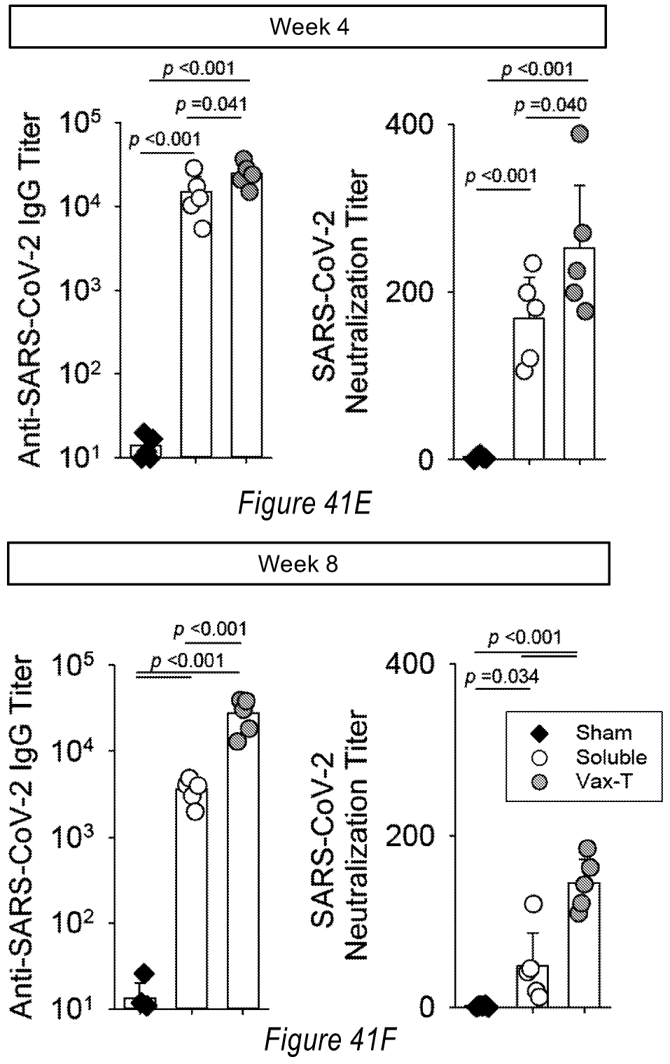

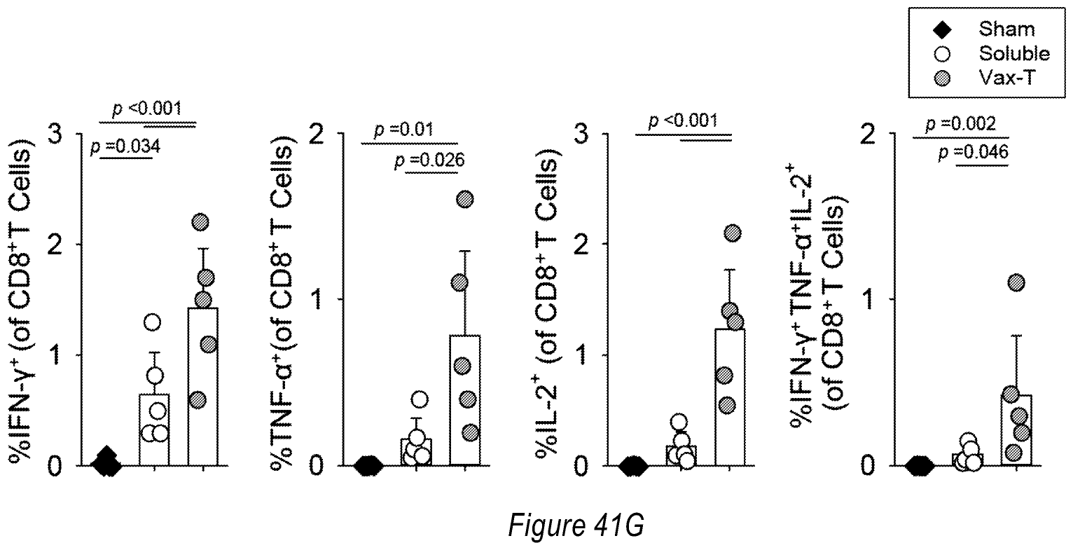

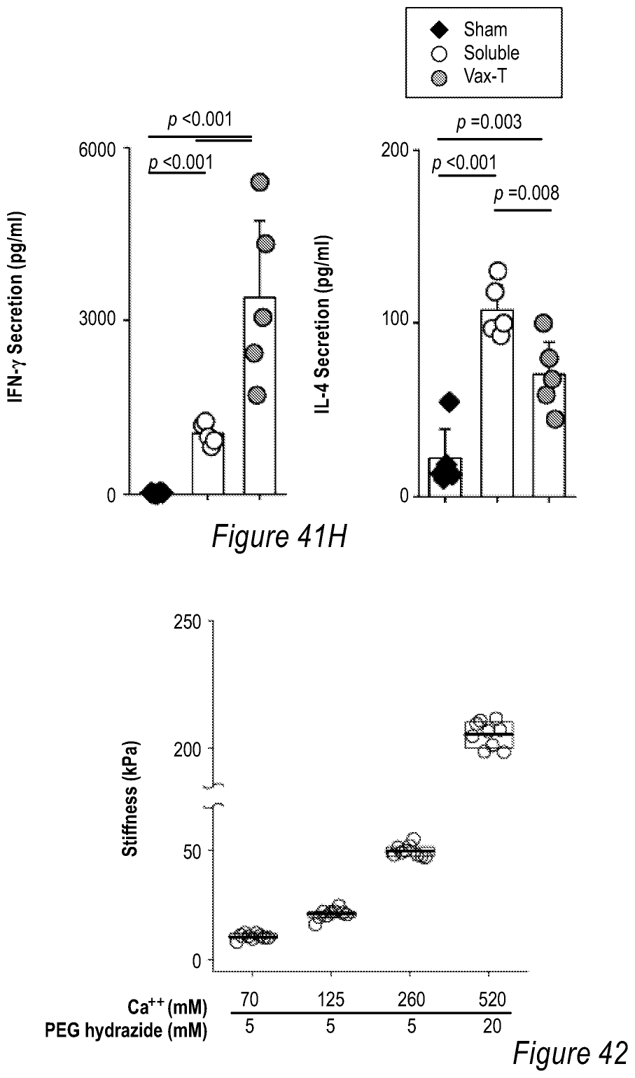

Vax-T Can Boost Vaccination Immunity in Immunocompromised Mice. Patients who undergo solid organ transplants and stem cell transplants are subjected to immunosuppressive medications that dramatically limit their ability to respond to vaccines. These immunocompromised patients face the highest risks of severe outcomes from infections including COVID-19. The ability of Vax-T, one of the most effective yet simplest microparticle-based vaccines, to elicit responses in immunocompromised mice was tested (FIG. 41A). A transplant model of xeno-mesenchymal stem cells (MSC) that allowed tracking survival and osteogenic regenerative potential over time was used. The osteogenic regeneration potential of MSCs is controlled by the interactions of MSCs with the recipient immune system. Four millions human bone marrow-derived MSCs mixed with spray-dried hydroxyapatite microparticles with an average size of 5 μm (1:1 ratio) were injected subcutaneously. In the absence of immunosuppression, the recipient immune response eliminated the osteogenic ability of the stem cells (FIG. 41B); however, osteogenesis was rescued by administration of T cell immunosuppressant drug Cyclosporine A (1 mg/kg/day). In vivo osteogenesis was validated using a micro-CT 3D and multiplanar images (FIG. 41C), confirming a significant level of immunosuppression was achieved. In this immunosuppressive context, the potential of Vax-T to elicit Spike peptide-specific responses was evaluated.

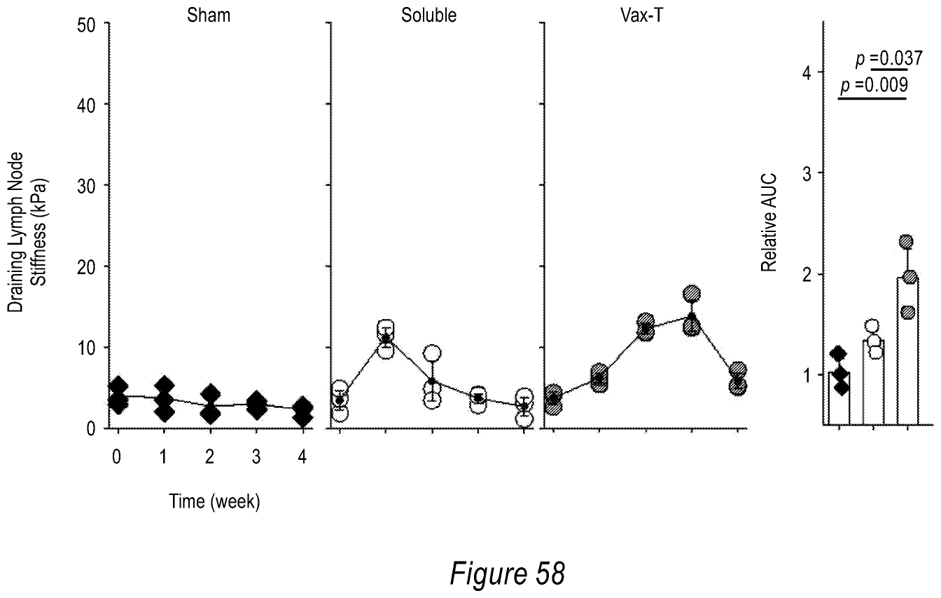

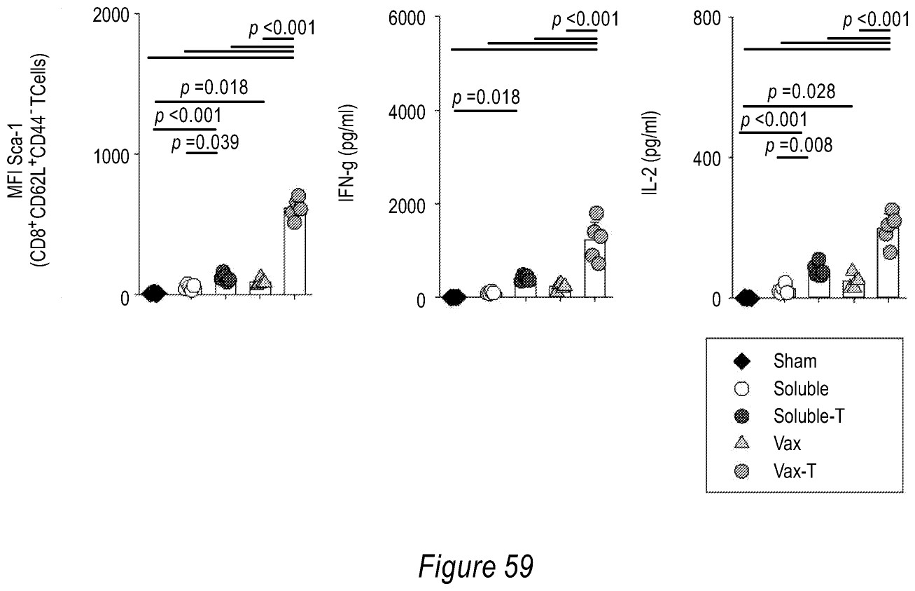

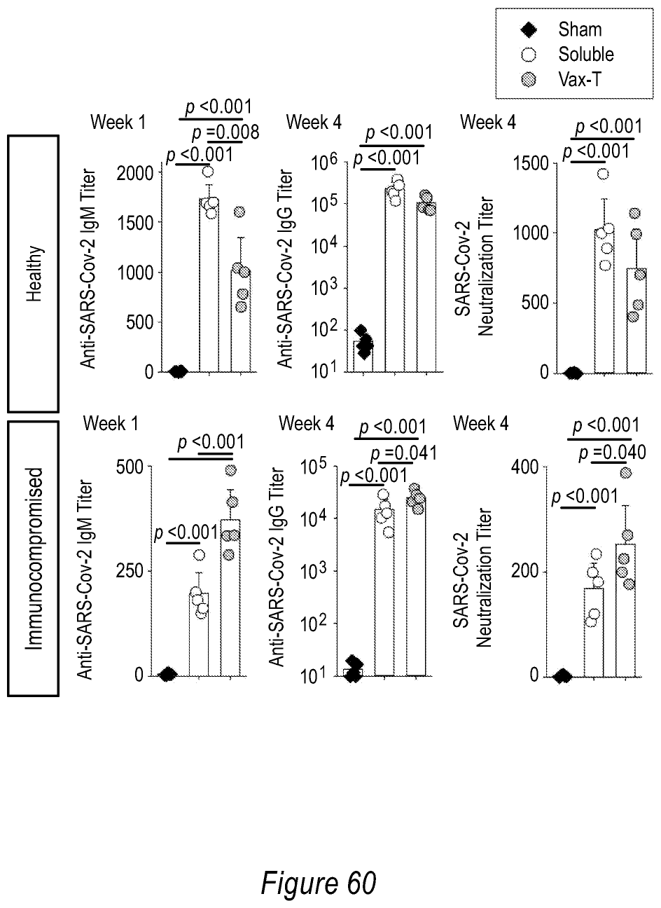

Mice were immunized as above, and dLN stiffening was evaluated first. Although the soluble vaccine caused much lower dLN stiffening in immunocompromised mice, Vax-T induced similar response as in healthy mice (Supplementary FIG. 26, FIG. 3a). Vax-T, but not soluble TWS119 injection, was able to induce TMSC formation in immunocompromised mice to the level about one-third of that in healthy mice (FIG. 58, FIG. 38B). Antibody responses were also evaluated from saphenous vein blood drawn at weeks 1, 4, and 8 after immunization. All immunocompromised mice receiving soluble vaccine and Vax-T had robust spike peptide-specific antibody (IgM and IgG) responses, and Vax-T outperformed soluble vaccine (FIGS. 41D-41F), although the antibody titers were lower than that in healthy mice (FIG. 60).

Production of neutralizing antibody was tested using SARS-CoV-2 surrogate and SARS-CoV-2 pseudotyped lentivirus neutralization assays as described above. All mice except those receiving mock immunization without spike peptides (sham), exhibited robust spike peptide-specific neutralizing antibody responses at week 4 post-vaccination (FIG. 41E). By 8 weeks, however, only the mice that received Vax-T generated detectable neutralizing antibodies (FIG. 41F).

To determine whether Vax-T could induce systematic T cell responses in immunocompromised mice, antigen-specific cytokine-production from T cells extracted from spleens 8 weeks after immunization was evaluated. Flow cytometry was used to measure cytokine production from CD8⁺ T cells. T cells were isolated and activated overnight with the SARS-CoV-2 Spike glycoprotein overlapping peptide pool. The results showed that one dose of Vax-T induced significantly more polyfunctional IFNγ/TNF/IL-2-producing T cells than two doses of soluble vaccine (FIG. 41G). ELISA showed that Vax-T induced higher level of pro-inflammatory IFNγ but lower level of anti-inflammatory IL-4 in immunocompromised mice, suggesting a Th1 type response (FIG. 41H). A similar trend was observed in peripheral blood, wherein the percent of CD8⁺ T cells producing the Type-1 cytokine IFN-γ was significantly higher for Vax-T treated mice compared with the soluble formulation (data not shown).

### Discussion

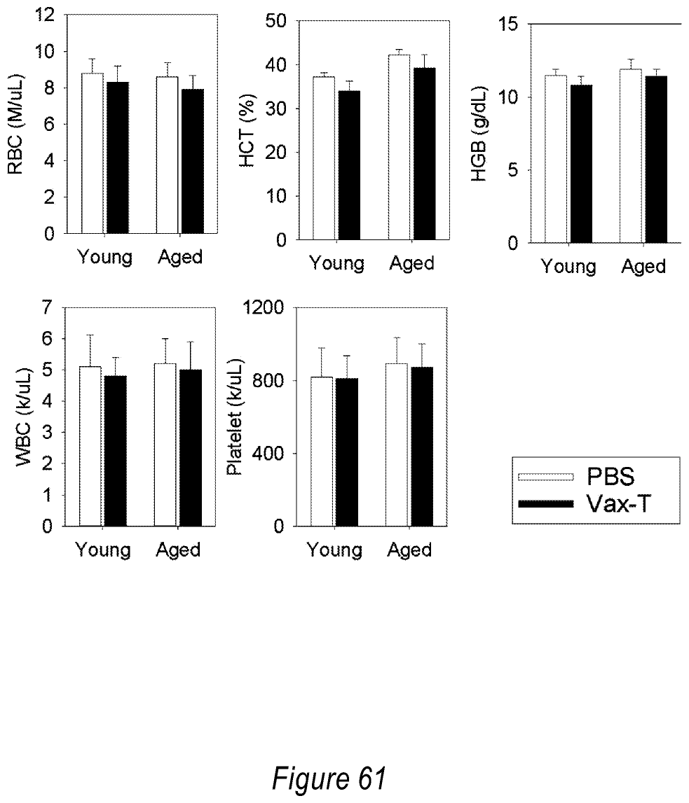

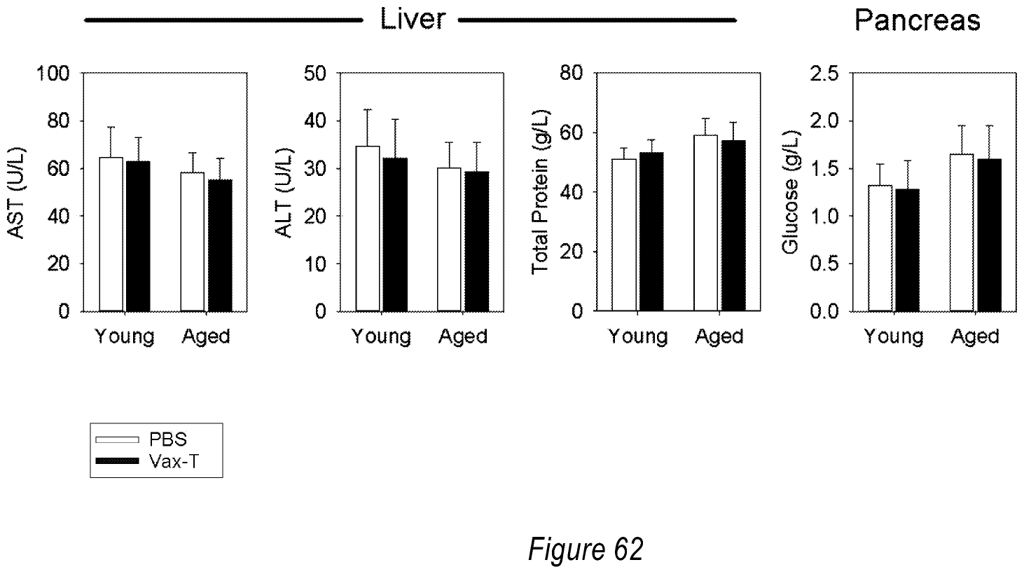

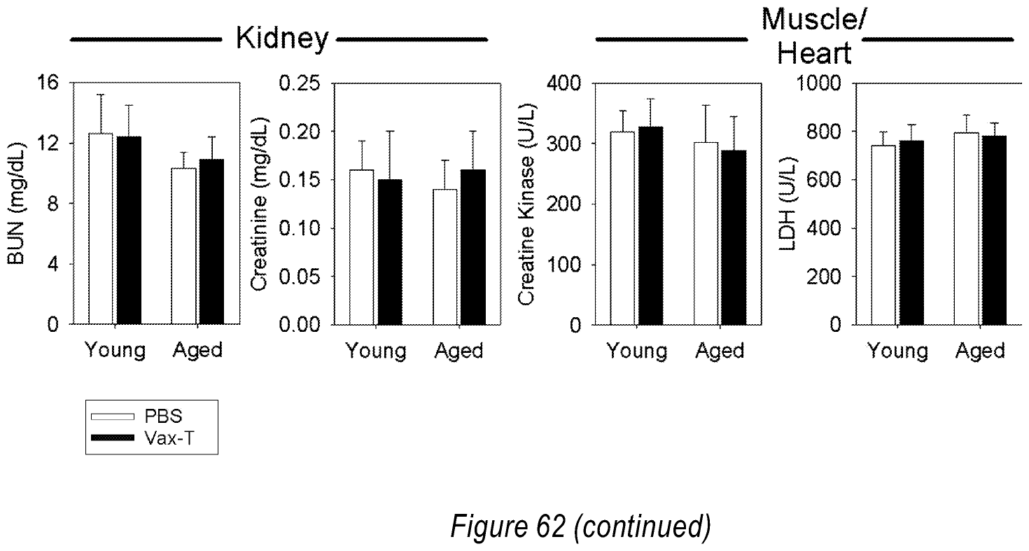

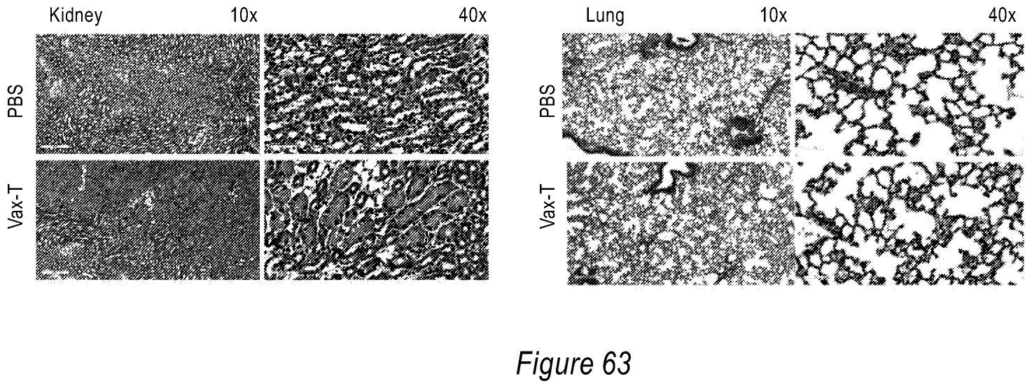

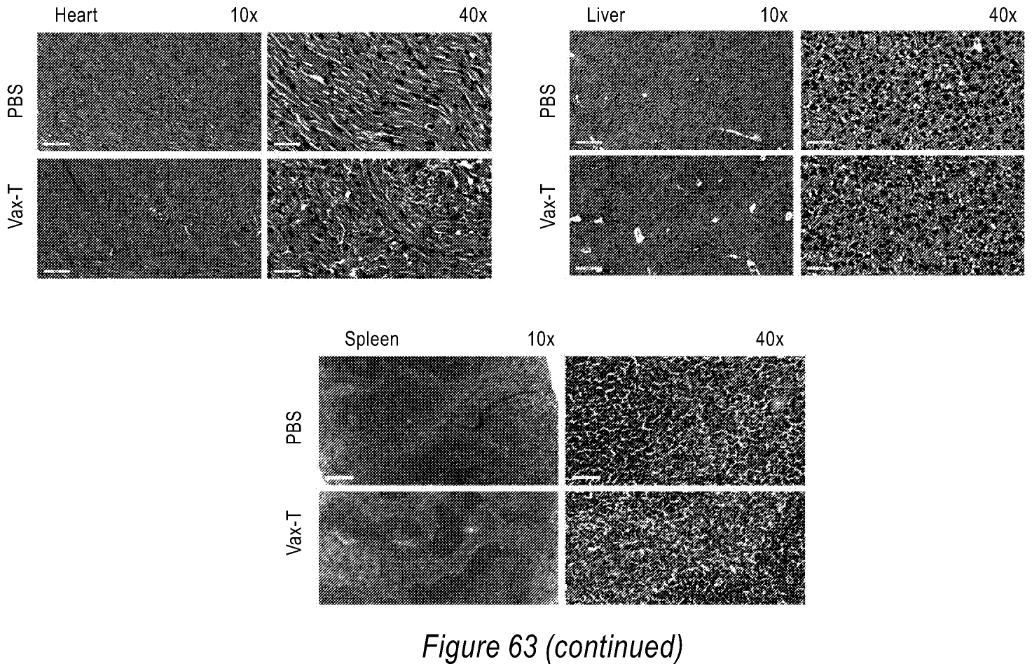

Aging and immunocompromised conditions hamper vaccine efficacy. The present solution to generate antigen-specific TMSC during vaccination offers a new approach to produce long-lasting T- and B-cell responses in the young, old, and immunocompromised. TMSC levels are low in patients who do not make long-lasting responses to Hepatitis B vaccine and yellow fever vaccine. Importantly, TMSC preferentially survive after the elimination of antigens and can stably persist for years, which can reconstitute an entire peripheral T cell population with a small number of cells. The present Vax-T design provided strong and prolonged immunity after a single administration of vaccine, not only in young and aged mice but also in immunosuppressed mice. It should be possible to not only deliver peptides as presented in this work, but also mRNA or attenuated viruses as alternate sources of vaccine antigens to foster specific TMSC development. The present work shows that vaccination strategies to elicit TMSC are viable and ready for translation to humans. Indeed, biocompatibility tests of Vax-T formulation, including hematological counts (FIG. 61), blood chemistry (FIG. 62) and histological analysis (FIG. 63) showed no toxicity.

Beyond vaccines, TMSC have begun to make an impact for engineered T cells in cancer immunotherapy because of their proliferative capacity, their ability to kill, and their vigorous production of cytokines. Manufacturing CAR-T cells out of TMSCS rather than conventional effector T cells could offer a potent immunotherapy. However, there has not been a reliable way to differentiate TMSC in vitro, except for the addition of IL-7 and IL-15 during stimulation with anti-CD3/CD28-coated beads. One important contribution of the present work was the identification that medium level of TCR triggering will induce formation of more TMSC than stronger TCR signals. This effect is similar to what was found for regulatory T cells (Tregs). In addition, the size and mechanical property of aAPC play an important role in TMSC induction. Furthermore, it was found that IL-7 and IL-15 only marginally increase the development of TMSC in the present system.

In vivo, activation of β-catenin by TWS119 that promotes the development of TMSC also diminished lymphoproliferation and lymphadenopathy that occurred after vaccination. The present data suggest that TCF1 or LEF1 expression could be sustained when TCR signal strength is low. When TCR signal strength is high, on the other hand, downregulation of TCR expression upon triggering would lead to diminished signaling over time and result in development of some TMSCs. The relative paucity during natural infections and cancers of TMSC as compared to T_{CM} probably owes to this need for dynamic moderation of signaling. It appears that microparticle-based Vax-T can induce TMSCs at both the injection site and in dLN, which is also supported by the observation that cells and TWS119-nanoparticles at the injection site could move to dLNs.

Many studies now support that TMSC play an important role in sustaining long-term memory. Recent studies suggest that TMSC are both numerically and functionally reduced in the elderly. For both CD4⁺ and CD8⁺ T cells, aging was associated with reduced β-catenin signaling due to an increase in circulating levels of DKK1, a natural inhibitor. This finding highlights the urgent need to exogenously boost TMSC during vaccination of the elderly, but at present there is no targeted approach to boost up formation of TMSC during vaccination. The Vax-T approach offered here presents a viable framework of antigens, adjuvants, and delivery modalities to endow protective prolonged TMSC-based immunity for the elderly and immunocompromised patients.

In summary, the biomaterial-based vaccines, as represented by Vax-T presented herein, enable a sustained release of vaccine components and induce a more potent immunization effect. The optimization of the biological, biochemical and biophysical properties of this vaccine platform will not only have broad applications for in vitro induction of TMSCs for T cell-based therapies but also help boost long-term immunity of vaccines for infectious diseases, cancer and other diseases.

### Example 9. Induction of Anti-Tumor Responses

In this study, cancer vaccination with Vax-CT (Vax-C+TWS119) promoted potent anti-tumoral T cell specific responses in mice against B16-F10 melanoma.

Tumor lysate is a source of antigens containing the full repertoire of the patient's specific tumor associated antigens, including neo-antigens. In this example, the incorporation of CpG (type B) was used to enhance the immunogenicity and activation of dendritic cells. Fresh B16F10 tumor was extracted from mice on day 15 post tumor injection. Tumor was manually processed into small fragments (˜1-3 mm3). The tumor fragments can be used fresh or following cryopreservation. The dissociation was performed in RPMI 1640 supplemented with DNase I at 30 IU/mL and collagenase at 0.3 PZ units/mL followed by an incubation at room temperature for 6 h. The lysate was then centrifuged, washed in PBS and a cell count was performed. The lysates were then exposed to a minimum of five freeze-thaw cycles. The samples can be stored at −80° C. until further use. Various concentrations of tumor lysates mixture were used during microparticles fabrication. Release of protein fragments was assessed by micro-BCA protein assay. The activity of tumor lysate-loaded microparticles was assessed using bone marrow dendritic cells (BMDCs). BMDCs isolated from the hind limb bones of C57BL/6 mice were cultivated in RPMI 1640 medium containing 10% fetal bovine serum, 1% penicillin and streptomycin, GM-CSF (20 ng/ml) and IL-4 (10 ng/ml) at 37° C. After being cultured for 6 days, the cells seeded in a 96-well plate at a density of 10⁴ cells per well and incubated overnight at 37° C. Subsequently, tumor lysate-loaded microparticles at different concentrations were added and co-incubated with the BMDCs for 12 h and the supernatants were analyzed for inflammatory cytokines (IL-6, IFN-g and TNF-α). The BMDCs were then harvested, and surface stained for CD40, MHC I, MHC II, CD80 and CD86 in the CD11c to be analyzed using flow cytometry.

To test the treatment outcome in vivo, B16 tumors grow to lethal size in ˜3 wks. During that time, tumor sizes were assessed by luciferase expression or caliber measurement. In other experiments, mice were euthanized on day 8, 15, and 22 to examine tumor infiltrating lymphocytes (TIL) penetration and exhaustion. Cellular response may be tested at various timepoints up to 12 weeks post-infusion for the surviving mice. In separate experiments, mice were studied to monitor survival.

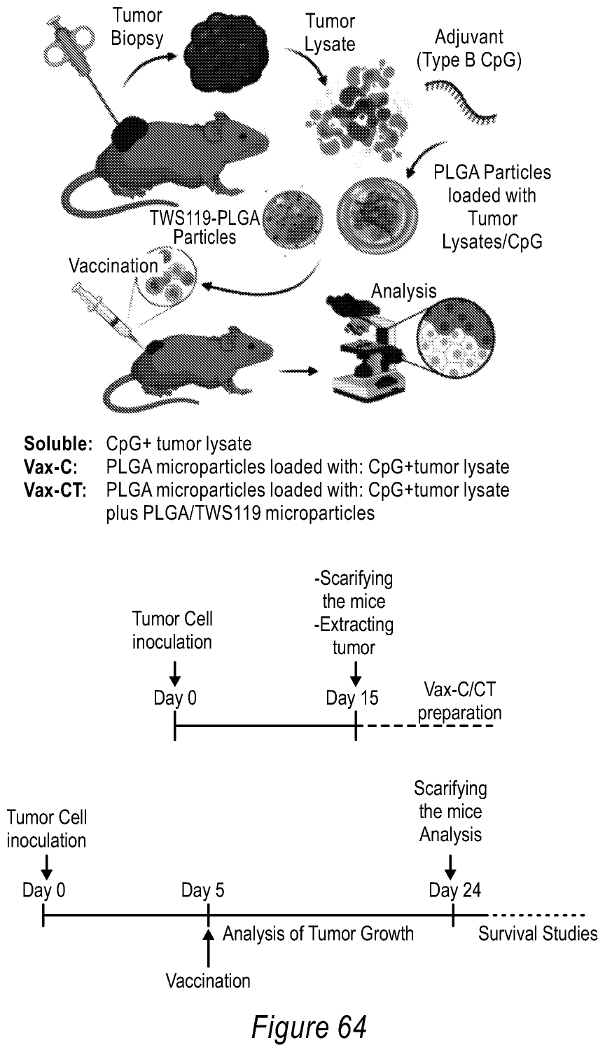

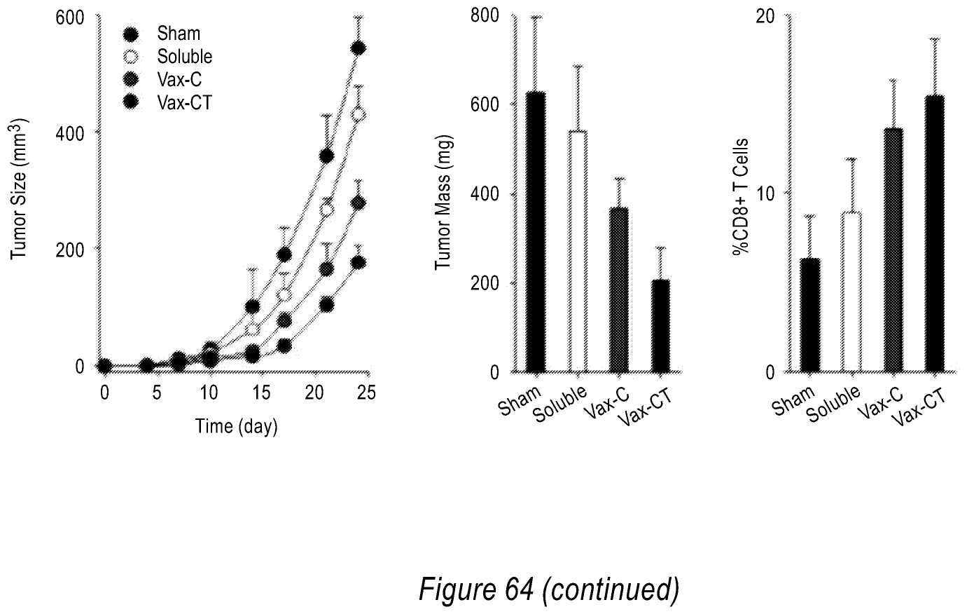

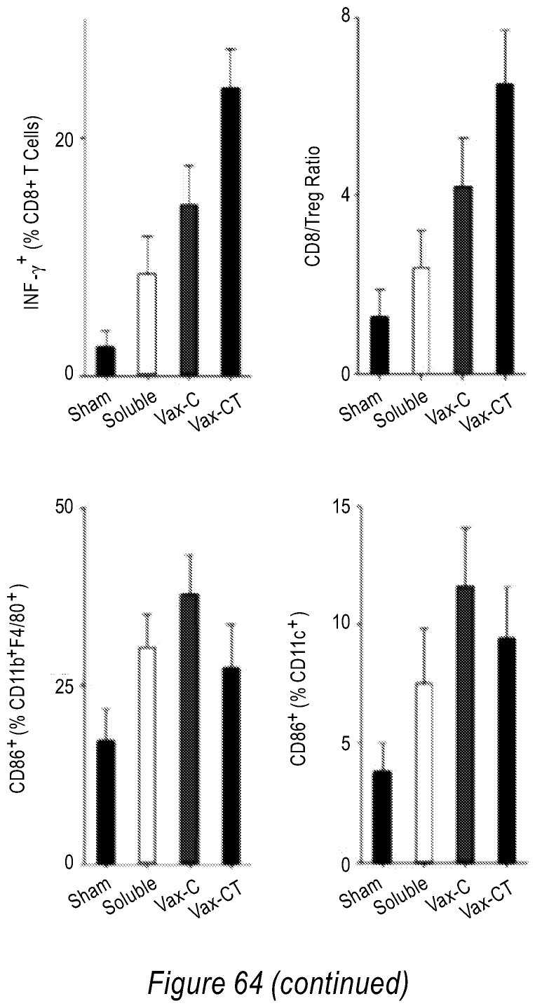

FIG. 64 shows cancer vaccination with Vax-CT promoted potent anti-tumoral T cell specific responses in mice against B16-F10 melanoma. Sham: Saline injection; Soluble: B16-F10 melanoma tumor lysate/IL-2/CpG; Vax-C: PLGA/B16-F10 melanoma lysate/IL-2/CpG microparticles (10 um); Vax-CT: Vax-T with TWS-119. IL-2: 100 U/injection; CpG 50 ug/injection; melanoma lysate 100 μg/injection. The presented data are expressed as mean±SD (n=5). The results were analyzed by using one-way ANOVA with post-hoc analysis. Statistical significance is indicated by the calculated p-values. Significant: p<0.05, very significant: p<0.01 and extremely significant: p<0.001.

### Example 10. Designed Artificial Antigen Presenting Cells can Outperform Dynabead and Provide More Powerful Tumor Fighting T Cells

In vitro assays, flow cytometry and intracellular cytokine assays, were used to test performance of designed artificial antigen presenting cells compared to Dynabead. Microparticles or dynabeads were incubated with murine/human primary T cells to study the effect of particle size and release rate on formation of TMSCs in vitro. The change in T cell activation, differentiation, and formation of t memory stem cells were monitored using flow cytometry by checking the surface markers including CD4, CD8, CD62L+, CD45RO−, CD45RA+, CD27+, CD95+, CD122+, and CCR7+ for human T cells and CD4, CD8, CD62L+, CD44−, CD122+, and Sca-1+ for murine T cells while expressing intracellular IFN-γ cytokine using intracellular cytokine staining.

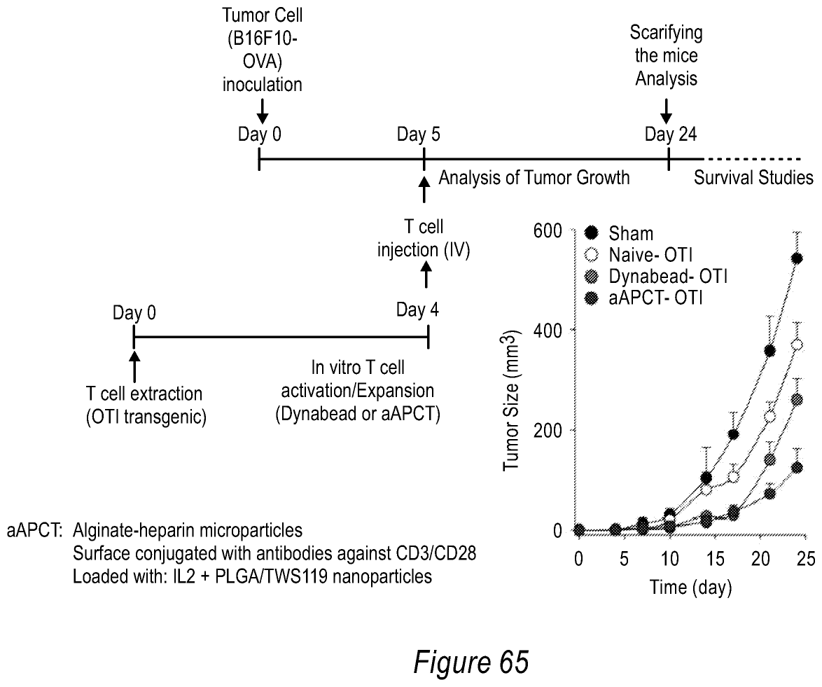

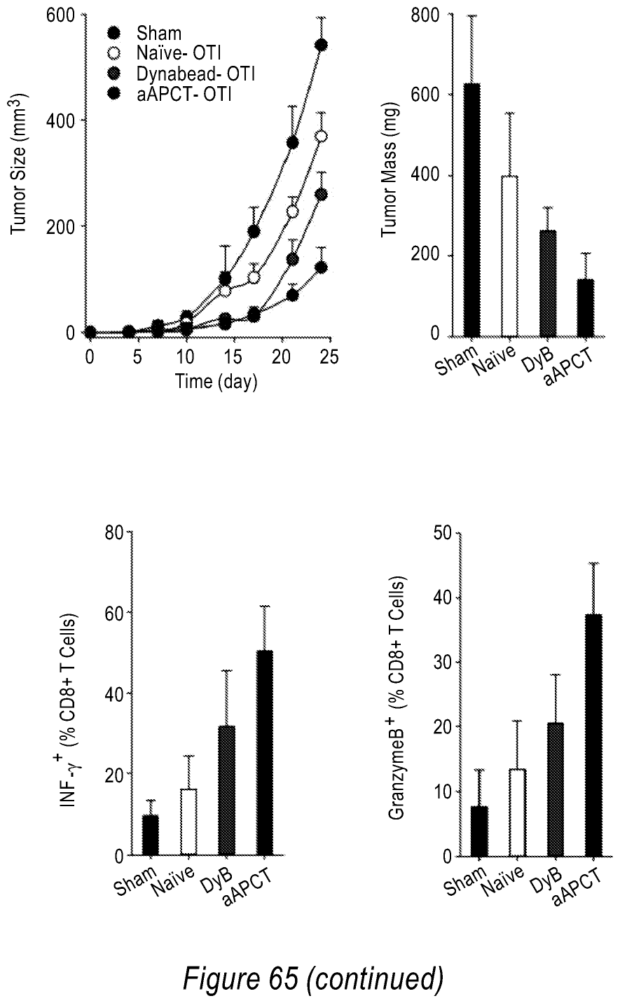

FIG. 65 shows designed artificial antigen presenting cells can outperform dynabead and provide more powerful tumor fighting T cells. aAPCT: alginate-heparin/IL-2 microparticles (5 um, 20 kPa) with medium level of anti-CD3/anti-CD28 surface antibodies with TWS-119 PLGA nanoparticles; IL-2: 100 U/injection. The presented data are expressed as mean±SD (n=5). The results were analyzed by using one-way ANOVA with post-hoc analysis. Statistical significance is indicated by the calculated p-values. Significant: p<0.05, very significant: p<0.01 and extremely significant: p<0.001.

What is claimed is:

1. A microparticle comprising:
(a) at least one immunogen; and
(b) at least one T memory stem cell (TMSC) inducer.

2. The microparticle of claim 1 wherein the immunogen is a viral, bacterial, protozoan, helminthic, or cancer antigen.

3. The method of claim 2 wherein the cancer antigen is from a solid tumor.

4. The microparticle of claim 1 wherein the at least one T memory stem cell (TMSC) inducer is TWS119.

5. The microparticle of claim 1 further comprising at least one immunostimulatory cytokine.

6. The microparticle of claim 5 wherein the at least one immunostimulatory cytokine is IL-2, IL-7 or IL-15, or any combination thereof.

7. The microparticle of claim 1 further comprising a T cell activator.

8. The microparticle of claim 7 wherein the T cell activator is anti-CD3 antibodies and/or anti-CD28 antibodies, or the combination thereof.

9. The microparticle of any one of claims 1-8 further comprising an adjuvant such as poly (I:C), CpG, alum, or any combination thereof.

10. The microparticle of any one of claims 1-8 further comprising an RGD peptide.

11. The microparticle of any one of claims 1-8 further comprising a chemokine.

12. The microparticle of claim 11 wherein the chemokine is CCL21, CCL19, CCL17, CCL22 or any combination thereof.

13. The microparticle of any one of claims 1-8 further comprising heparin.

14. The microparticle of any one of claims 1-8 comprising a polymer scaffold such as alginate, hyaluronic acid, polycaprolactone, poly(lactic acid), or PLGA.

15. The microparticle of claim 14 wherein the alginate is cross-linked with calcium.

16. The microparticle of claim 1 wherein the TMSC inducer is provided in a nanoparticle within the microparticle.

17. The microparticle of claim 16 wherein the nanoparticle comprises PLGA.

18. The microparticle of claim 1, wherein the microparticles comprise a porous scaffold.

19. The microparticle of claim 1, wherein the porous scaffold comprises anti-CD3 or anti-CD28 antibodies bound to or cross-linked to heparin.

20. The microparticle of claim 19, wherein the cross-link is by NHS/EDC.

21. The microparticle of claim 1, wherein microparticles comprise RGD peptide conjugated to alginate via NHS/EDC.

22. The microparticle of claim 1, wherein the microparticles have an average size of about 0.4 μm to about 50 μm.

23. The microparticle of claim 1, wherein the microparticles have an average size of about 200 μm to about 800 μm.

24. The microparticle of claim 23, wherein the microparticles have average pore size of about 20 μm to about 100 μm.

25. The microparticle of claim 1, wherein the microparticles comprise a stiffness of from about 0.5-250 kPa.

26. A pharmaceutical composition comprising the microparticles of any one of claims 1-25.

27. The pharmaceutical composition of claim 26 comprising a vehicle, excipient, vehicle, adjuvant, immune cells, or any combination thereof.

28. The pharmaceutical composition of claim 26 wherein the adjuvant is alum, CpG, poly (I:C), or any combination thereof.

29. The pharmaceutical composition of claim 26 formulated for subcutaneous injection.

30. A method for immunizing a subject to an antigen comprising administering to the subject a pharmaceutical composition of any one of claims 26-29 or a microparticle of any one of claims 1-25.

31. The method of claim 30 wherein the subject is elderly, has immunosenescence, is immunocompromised, or is undergoing cancer therapy.

32. The method of claim 30 wherein the immunizing is for therapeutic use.

33. The method of claim 30 wherein the immunizing is for preventative use.
